(19) United States
(12) Patent Application Publication
(10) Pub. No.: US 2025/0136686 A1
(43) Pub. Date: May 1, 2025

(54) ANTI-CD101 ANTIBODY AND USE THEREOF

(71) Applicant: GUANGDONG KEGUANDA PHARMACEUTICAL TECHNOLOGY CO., LTD., Guangdong (CN)

(72) Inventors: Hung Yan Lau, Guangdong (CN); Chin Yu Li, Guangdong (CN)

(21) Appl. No.: 19/006,217

(22) Filed: Dec. 30, 2024

Related U.S. Application Data
(63) Continuation of application No. PCT/CN2023/097142, filed on May 30, 2023.

(30) Foreign Application Priority Data
Jul. 1, 2022 (CN) 202210774689.8

(51) Int. Cl. C07K 16/28; A61K 39/00; A61P 35/00

(52) U.S. Cl. CPC C07K 16/2803; A61P 35/00; A61K 2039/505; C07K 2317/24; C07K 2317/76; C07K 2317/92

(57) ABSTRACT

The present disclosure provides a method for treating a disease with an inhibitor for inhibiting expression and/or activity of CD101, and in particular a method for treating cancer, an inflammatory or infectious disease, an autoimmune disease, or an endocrine disease. The present disclosure also provides an antibody specifically binding to CD101, a nucleic acid encoding the antibody, a vector including the nucleic acid, and a host cell including the nucleic acid or the vector. The present disclosure also provides a pharmaceutical composition including the antibody, and a use of the antibody and the pharmaceutical composition.

### CROSS-REFERENCE TO RELATED APPLICATIONS

The present application is a continuation application of PCT application No. PCT/CN2023/097142 filed on May 30, 2023, which claims the benefit of Chinese Patent Application No. 202210774689.8 filed on Jul. 1, 2022. The contents of all of the aforementioned applications are incorporated by reference herein in their entirety.

### TECHNICAL FIELD

The present disclosure relates to a method for treating a disease with an inhibitor for inhibiting expression and/or activity of CD101, and in particular to a method for treating cancer, an inflammatory or infectious disease, an autoimmune disease, or an endocrine disease. The present disclosure also provides an antibody specifically binding to CD101, and a use of the antibody.

### REFERENCE TO SEQUENCE LISTING

The Sequence Listing XML file submitted via the USPTO Patent Center, with a file name of “Sequence listing_SCH-24110-USPT.xml”, a creation date of Dec. 30, 2024, and a size of 20,398 bytes, is part of the specification and is incorporated in its entirety by reference herein.

### BACKGROUND

According to the latest national cancer statistics released by the National Cancer Center, cancer has become one of the major public health problems that seriously threaten the health of Chinese populations, deaths caused by malignant tumors account for 23.91% of all deaths in residents, and the incidence and mortality of cancer continue to increase. In recent years, the immunotherapy research has been highly anticipated, and inhibitors for programmed cell death protein 1 (PD-1) and ligands of PD-1 (PD-L1) have been approved by the Food and Drug Administration (FDA) for separate or combined first-line therapy. These methods have significantly improved the survival rate for patients with some types of advanced cancer. However, the prominent drawback is that a patient has a low response rate to αPD-1 monoclonal antibody therapy, and in particular, the αPD-1 monoclonal antibody therapy exhibits an effective rate of less than 20% to 30% for patients with lung cancer.

CD101 is a surface antigen of human leukocytes, which is a transmembrane protein composed of 1,021 amino acids. CD101 is expressed on the surface of activated T cells, dendritic cells, Langerhans cells, etc. CD101 has the effect of inhibiting the activation and proliferation of T cells. T cells in patients with cancer and chronic infection are at the irreversible stage of terminal differentiation, which means that CD101 is the last link of T cell exhaustion. CD101 can inhibit the expression of interleukin 2 receptor alpha (IL2RA) on activated T cells and the secretion of interleukin-2 (IL-2), and can also inhibit the proliferation of T cells by stimulating the secretion of IL10 by dendritic cells.

CD101 is closely associated with the progression of many diseases, such as acute lung injury (ALI), inflammatory bowel disease (IBD), and type 1 diabetes (T1D). ALI is a fatal disease lacking therapeutic drugs, and is accompanied with severe neutrophil inflammation. Animal experiments have shown that allergic CD101⁺ eosinophils can exacerbate the lipopolysaccharide (LPS)-induced neutrophil inflammation and aggravate the lung injury, indicating that CD101 is a potential therapeutic target for ALI. CD101 has been detected on intraepithelial lymphocytes (IELs) and regulatory T cells (Tregs) in the intestinal tract, indicating that CD101 is involved in the occurrence and progression of IBD and T1D. Other studies have shown that the metastasis of CD101⁺ T cells can lead to severe colitis, which may be related to the expansion of IL-17-secreting T cells and the enhancement of expression of IL-2Ra/b. The association between CD101 and T1D susceptibility is related to functions of CD101⁺ Tregs and antigen-presenting cells. In addition, in the synovial fluid (SF) of patients with rheumatoid arthritis (RA), tissue-resident memory T (TRM) cells increase and are mainly composed of CD45RA memory T cells with CD69 and/or CD103, and the expression of CD101 on TRM is upregulated, suggesting that CD101 is involved in the perform-mediated citrullination and the onset of RA. Additional studies have shown that the genetic variation of CD101 can significantly increase the risk of sexually acquired human immunodeficiency virus (HIV)-1 infection. CD101 can reduce an ability of Tregs to inhibit the cytokine production of T cells to decrease the baseline level of immunoquiescence, which in turn increases the possibility of HIV-1 infection. Therefore, CD101 is considered a potential drug target against HIV-1 infection.

### SUMMARY

The present disclosure provides a method for treating a disease with an inhibitor for inhibiting expression and/or activity of CD101, and in particular a method for treating cancer, an inflammatory or infectious disease, an autoimmune disease, or an endocrine disease. The present disclosure also provides an antibody specifically binding to CD101, a nucleic acid encoding the antibody, a vector including the nucleic acid, and a host cell including the nucleic acid or the vector. The present disclosure also provides a pharmaceutical composition including the antibody, and a use of the antibody and the pharmaceutical composition.

Accordingly, in an aspect, the present disclosure provides a method for treating a disease in a subject, including administering an inhibitor for inhibiting expression and/or activity of CD101 to the subject, where the disease is selected from the group consisting of cancer, an inflammatory or infectious disease, an autoimmune disease, or an endocrine disease.

In some embodiments of the method in the present disclosure, the cancer is selected from the group consisting of a hematologic cancer and a solid tumor; optionally, the hematologic cancer is selected from the group consisting of leukemia, polycythemia vera, lymphoma, multiple myeloma, Waldenstrom macroglobulinemia, or heavy chain diseases; and optionally, the solid tumor is selected from the group consisting of lung cancer, melanoma, ovarian cancer, pancreatic ductal adenocarcinoma, liver cancer, colorectal cancer, glioblastoma, or prostate cancer. In some embodiments, the cancer is non-small cell lung cancer (NSCLC). In some embodiments, the subject does not respond to treatment with a PD-1 axis inhibitor.

In some embodiments of the method in the present disclosure, the inflammatory or infectious disease is selected from the group consisting of ALI, IBD, and HIV infection.

In some embodiments of the method in the present disclosure, the autoimmune disease or the endocrine disease is selected from the group consisting of RA and T1D.

In another aspect, the present disclosure provides an inhibitor for inhibiting expression and/or activity of CD101, where the inhibitor is provided to treat cancer, an inflammatory or infectious disease, an autoimmune disease, or an endocrine disease in a subject.

In another aspect, the present disclosure provides a use of the inhibitor for inhibiting expression and/or activity of CD101 in preparation of a drug for treating cancer, an inflammatory or infectious disease, an autoimmune disease, or an endocrine disease in a subject.

In some embodiments of the above aspect, the inhibitor for inhibiting expression and/or activity of CD101 is an antibody specifically binding to CD101 or an antigen-binding fragment thereof.

In some embodiments, the antibody specifically binding to CD101 or the antigen-binding fragment thereof includes VH and VL, where
(i) the VH includes CDR-H1, CDR-H2, and CDR-H3 having amino acid sequences set forth in SEQ ID NOs: 1-3, respectively, and the VL includes CDR-L1, CDR-L2, and CDR-L3 having an amino acid sequence set forth in SEQ ID NO: 5, an amino acid sequence QMS, and an amino acid sequence set forth in SEQ ID NO: 6, respectively; or
(ii) the VH includes CDR-H1, CDR-H2, and CDR-H3 having amino acid sequences set forth in SEQ ID NOs: 8-10, respectively, and the VL includes CDR-L1, CDR-L2, and CDR-L3 having an amino acid sequence set forth in SEQ ID NO: 12, an amino acid sequence AAT, and an amino acid sequence set forth in SEQ ID NO: 13, respectively.

In some embodiments,
(i) the VH includes an amino acid sequence having at least 80%, at least 85%, at least 90%, at least 95%, at least 98%, at least 99%, or 100% sequence identity to SEQ ID NO: 4, and the VL includes an amino acid sequence having at least 80%, at least 85%, at least 90%, at least 95%, at least 98%, at least 99%, or 100% sequence identity to SEQ ID NO: 7; or
(ii) the VH includes an amino acid sequence having at least 80%, at least 85%, at least 90%, at least 95%, at least 98%, at least 99%, or 100% sequence identity to SEQ ID NO: 11, and the VL includes an amino acid sequence having at least 80%, at least 85%, at least 90%, at least 95%, at least 98%, at least 99%, or 100% sequence identity to SEQ ID NO: 14.

In some embodiments,
(i) the VH includes an amino acid sequence set forth in SEQ ID NO: 4, and the VL includes an amino acid sequence set forth in SEQ ID NO: 7; or
(ii) the VH includes an amino acid sequence set forth in SEQ ID NO: 11, and the VL includes an amino acid sequence set forth in SEQ ID NO: 14.

In some embodiments, the VH includes an amino acid sequence having at least 80%, at least 85%, at least 90%, at least 95%, at least 98%, at least 99%, or 100% sequence identity to an amino acid sequence set forth in any one of SEQ ID NOs: 15-18, and the VL includes an amino acid sequence having at least 80%, at least 85%, at least 90%, at least 95%, at least 98%, at least 99%, or 100% sequence identity to an amino acid sequence set forth in any one of SEQ ID NOs: 19-21.

In some embodiments, the VH includes an amino acid sequence set forth in any one of SEQ ID NOs: 15-18, and the VL includes an amino acid sequence set forth in any one of SEQ ID NOs: 19-21.

In a preferred embodiment,
(i) the VH includes an amino acid sequence set forth in SEQ ID NO: 15, and the VL includes an amino acid sequence set forth in SEQ ID NO: 19; or
(ii) the VH includes an amino acid sequence set forth in SEQ ID NO: 15, and the VL includes an amino acid sequence set forth in SEQ ID NO: 20; or
(iii) the VH includes an amino acid sequence set forth in SEQ ID NO: 17, and the VL includes an amino acid sequence set forth in SEQ ID NO: 19; or
(iv) the VH includes an amino acid sequence set forth in SEQ ID NO: 17, and the VL includes an amino acid sequence set forth in SEQ ID NO: 20.

In some embodiments of the above aspect, the antibody can be selected from the group consisting of IgG, IgA, IgM, IgE, and IgD isotypes, such as IgG1, IgG2, IgG3, and IgG4 subtypes.

In some embodiments, the antibody can be selected from the group consisting of a murine antibody, a chimeric antibody, a humanized antibody, and a fully human antibody.

In some embodiments, the antigen-binding fragment can be selected from the group consisting of Fab, Fab′, F(ab′)₂, Fv, scFv, and ds-scFv.

In some embodiments, the antibody can be a bispecific antibody or a multispecific antibody.

In some embodiments of the above aspects, the method further includes administering a second therapeutic agent to the subject, or the inhibitor for inhibiting expression and/or activity of CD101 is further used in combination with the second therapeutic agent, or the drug further includes the second therapeutic agent, where the second therapeutic agent is selected from the group consisting of an antibody, a chemotherapeutic agent, and a small molecule drug.

In some embodiments, the second therapeutic agent is selected from the group consisting of an antibiotic derivative, a platinum complex, paclitaxel, camptothecin, an antimetabolite, a folic acid analogue, a purine analogue, a pyrimidine analogue, an antiproliferative/antimitotic agent, an alkylating agent, a DNA damaging agent, an antiestrogen, an antiandrogen, an aromatase inhibitor, a hormone, a corticosteroid, a nitrogen mustard derivative, a steroid and a combination thereof, an antiplatelet drug, an antimetastatic agent, an antisecretory agent, an antiadrenergic agent, a growth factor inhibitor, a fibroblast growth factor inhibitor, an angiotensin receptor blocker, a nitric oxide donor, a cell cycle inhibitor, a topoisomerase inhibitor, a kinase inhibitor of growth factor signal transduction, a dysfunction inducer, a toxin, a fibrinolytic agent, an immunosuppressant, cycomelamine and melamine, bryostatin, kallistatin, an anti-angiogenic agent, a tissue inhibitor of metalloprotease-1, a tissue inhibitor of metalloprotease-2, a plasminogen activator inhibitor-1, a plasminogen activator inhibitor-2, a cartilage-derived inhibitor, a platelet factor 4, protamine sulfate, a sulfated chitin derivative (prepared from a hairy crab shell), a sulfated polysaccharide-peptidoglycan complex (SP-PG), staurosporine, a regulator of matrix metabolism, an antibody, a cytokine, a therapeutic radiopharmaceutical agent, and a combination thereof.

In another aspect, the present disclosure provides an antibody specifically binding to CD101 or an antigen-binding fragment thereof, including VH and VL, where
(i) the VH includes CDR-H1, CDR-H2, and CDR-H3 having amino acid sequences set forth in SEQ ID NOs: 1-3, respectively, and the VL includes CDR-L1, CDR-L2, and CDR-L3 having an amino acid sequence set forth in SEQ ID NO: 5, an amino acid sequence QMS, and an amino acid sequence set forth in SEQ ID NO: 6, respectively; or
(ii) the VH includes CDR-H1, CDR-H2, and CDR-H3 having amino acid sequences set forth in SEQ ID NOs: 8-10, respectively, and the VL includes CDR-L1, CDR-L2, and CDR-L3 having an amino acid sequence set forth in SEQ ID NO: 12, an amino acid sequence AAT, and an amino acid sequence set forth in SEQ ID NO: 13, respectively.

In some embodiments of the antibody or the antigen-binding fragment thereof in the present disclosure,
(i) the VH includes an amino acid sequence having at least 80%, at least 85%, at least 90%, at least 95%, at least 98%, at least 99%, or 100% sequence identity to SEQ ID NO: 4, and the VL includes an amino acid sequence having at least 80%, at least 85%, at least 90%, at least 95%, at least 98%, at least 99%, or 100% sequence identity to SEQ ID NO: 7; or
(ii) the VH includes an amino acid sequence having at least 80%, at least 85%, at least 90%, at least 95%, at least 98%, at least 99%, or 100% sequence identity to SEQ ID NO: 11, and the VL includes an amino acid sequence having at least 80%, at least 85%, at least 90%, at least 95%, at least 98%, at least 99%, or 100% sequence identity to SEQ ID NO: 14.

In some embodiments,
(i) the VH includes an amino acid sequence set forth in SEQ ID NO: 4, and the VL includes an amino acid sequence set forth in SEQ ID NO: 7; or
(ii) the VH includes an amino acid sequence set forth in SEQ ID NO: 11, and the VL includes an amino acid sequence set forth in SEQ ID NO: 14.

In some embodiments of the antibody or the antigen-binding fragment thereof in the present disclosure, the VH includes an amino acid sequence having at least 80%, at least 85%, at least 90%, at least 95%, at least 98%, at least 99%, or 100% sequence identity to an amino acid sequence set forth in any one of SEQ ID NOs: 15-18, and the VL includes an amino acid sequence having at least 80%, at least 85%, at least 90%, at least 95%, at least 98%, at least 99%, or 100% sequence identity to an amino acid sequence set forth in any one of SEQ ID NOS: 19-21.

In some embodiments, the VH includes an amino acid sequence set forth in any one of SEQ ID NOs: 15-18, and the VL includes an amino acid sequence set forth in any one of SEQ ID NOs: 19-21.

In a preferred embodiment,
(i) the VH includes an amino acid sequence set forth in SEQ ID NO: 15, and the VL includes an amino acid sequence set forth in SEQ ID NO: 19; or
(ii) the VH includes an amino acid sequence set forth in SEQ ID NO: 15, and the VL includes an amino acid sequence set forth in SEQ ID NO: 20; or
(iii) the VH includes an amino acid sequence set forth in SEQ ID NO: 17, and the VL includes an amino acid sequence set forth in SEQ ID NO: 19; or
(iv) the VH includes an amino acid sequence set forth in SEQ ID NO: 17, and the VL includes an amino acid sequence set forth in SEQ ID NO: 20.

In some embodiments of the antibody or the antigen-binding fragment thereof in the present disclosure, the antibody can be selected from the group consisting of IgG, IgA, IgM, IgE, and IgD isotypes, such as IgG1, IgG2, IgG3, and IgG4 subtypes.

In some embodiments, the antibody can be selected from the group consisting of a murine antibody, a chimeric antibody, a humanized antibody, and a fully human antibody.

In some embodiments, the antigen-binding fragment can be selected from the group consisting of Fab, Fab′, F(ab′)₂, Fv, scFv, and ds-scFv.

In some embodiments, the antibody can be a bispecific antibody or a multispecific antibody.

In another aspect, the present disclosure provides a nucleic acid including a nucleotide sequence encoding the antibody or the antigen-binding fragment thereof of the present disclosure.

In yet another aspect, the present disclosure provides a vector including a nucleotide sequence encoding the antibody or the antigen-binding fragment thereof of the present disclosure.

In yet another aspect, the present disclosure provides a host cell including the nucleic acid of the present disclosure or the vector of the present disclosure.

In yet another aspect, the present disclosure provides a pharmaceutical composition, including: the antibody or the antigen-binding fragment thereof of the present disclosure, and a pharmaceutically acceptable carrier or excipient.

In some embodiments of the pharmaceutical composition in the present disclosure, the pharmaceutical composition further includes a second therapeutic agent. In some embodiments, the second therapeutic agent can be selected from the group consisting of an antibody, a chemotherapeutic agent, and a small molecule drug. In some embodiments, the second therapeutic agent can be selected from the group consisting of an antibiotic derivative, a platinum complex, paclitaxel, camptothecin, an antimetabolite, a folic acid analogue, a purine analogue, a pyrimidine analogue, an antiproliferative/antimitotic agent, an alkylating agent, a DNA damaging agent, an antiestrogen, an antiandrogen, an aromatase inhibitor, a hormone, a corticosteroid, a nitrogen mustard derivative, a steroid and a combination thereof, an antiplatelet drug, an antimetastatic agent, an antisecretory agent, an antiadrenergic agent, a growth factor inhibitor, a fibroblast growth factor inhibitor, an angiotensin receptor blocker, a nitric oxide donor, a cell cycle inhibitor, a topoisomerase inhibitor, a kinase inhibitor of growth factor signal transduction, a dysfunction inducer, a toxin, a fibrinolytic agent, an immunosuppressant, cycomelamine and melamine, bryostatin, kallistatin, an anti-angiogenic agent, a tissue inhibitor of metalloprotease-1, a tissue inhibitor of metalloprotease-2, a plasminogen activator inhibitor-1, a plasminogen activator inhibitor-2, a cartilage-derived inhibitor, a platelet factor 4, protamine sulfate, a sulfated chitin derivative (prepared from a hairy crab shell), a sulfated polysaccharide-peptidoglycan complex (SP-PG), staurosporine, a regulator of matrix metabolism, an antibody, a cytokine, a therapeutic radiopharmaceutical agent, and a combination thereof.

### BRIEF DESCRIPTION OF THE DRAWINGS

The comprehension of the features and advantages of the present application can be acquired with reference to the following detailed description and accompanying drawings describing the exemplary embodiments utilizing the principle of the present application.

FIG. 1A and FIG. 1B show fluorescence-activated cell sorting (FACS) results for preparation of CHO-S-CD101 and 293F-CD101 expression cell lines, where negative control (NC) is phosphate buffered saline (PBS) and control cells are CHO-S (FIG. 1A) and 293F (FIG. 1B);

FIG. 2 shows FACS results for mouse serum samples during mouse immunization, where NC is PBS;

FIG. 3 shows schematic structural diagrams of pcDNA3.4-IgG1 and pcDNA3.4-IgKc expression vectors;

FIG. 4 shows FACS results for chimeric antibodies, where positive control (PC) is a positive serum diluted at 1:1,000 and NC is PBS;

FIG. 5 shows FACS results for purified chimeric antibodies;

FIG. 6 shows FACS results for humanized antibodies;

FIG. 7 shows sodium dodecyl sulfate-polyacrylamide gel electrophoresis (SDS-PAGE) results for humanized purified antibodies, where the lane M is for the protein marker; in the left panel, the lane 1 is for the hu.CA1 antibody and the lane 2 is for the hu.CA2 antibody; and in the right panel, the lane 1 is for the 19-A11-H3-B11 chimeric antibody (CA1), the lane 2 is for the hu.CA3 antibody, and the lane 3 is for the hu.CA4 antibody;

FIG. 8 shows FACS results for humanized purified antibodies, where the control is the 19-A11-H3-B11 chimeric antibody (CA1);

FIG. 9 shows FACS results and EC₅₀ values for humanized purified antibodies, where the control is the 19-A11-H3-B11 chimeric antibody (CA1);

FIG. 10 shows FACS results for preparation of a stable Jurkat cell line overexpressing CD101;

FIG. 11A to FIG. 11J show that an anti-CD101 chimeric antibody (CA1, FIG. 11A to FIG. 11B) and humanized antibodies (hu.CA1 to hu.CA4, FIG. 11C to FIG. 11J) all exhibit significant affinity and inhibition for CD101 at different concentrations and under different action times;

FIG. 12 shows the expression of IL-2 in lentivirus-transfected Jurkat cells overexpressing CD101 treated with a chimeric antibody (CA1) and humanized antibodies (hu. CA1 to hu.CA4) at 1,600 ng/mL for 8 h, where “normal cells” are untransfected normal Jurkat cells, and “lentivirus NC” and “lentivirus overexpressing CD101” corresponded to stable Jurkat cells infected with an NC lentivirus PMT290 and a lentivirus PSE5799 overexpressing human CD101, respectively;

FIG. 13 shows changes in body weights of mice in PBS, anti-CD101 antibody, and anti-PD-1 antibody administration groups among tumor-inoculated model mice over time;

FIG. 14 shows changes in tumor volumes of mice in PBS, anti-CD101 antibody, and anti-PD-1 antibody administration groups among tumor-inoculated model mice over time, where *P<0.05, **P<0.01 and ***P<0.0001;

FIG. 15 shows images of tumors collected from mice in PBS, anti-CD101 antibody, and anti-PD-1 antibody administration groups among tumor-inoculated model mice on D18;

FIG. 16 shows weights of tumors collected from mice in PBS, anti-CD101 antibody, and anti-PD-1 antibody administration groups among tumor-inoculated model mice on D18; and

FIG. 17 shows organ coefficients of organs collected from mice in PBS, anti-CD101 antibody, and anti-PD-1 antibody administration groups among tumor-inoculated model mice on D18.

### DETAILED DESCRIPTION

The present disclosure provides a method for treating a disease with an inhibitor for inhibiting expression and/or activity of CD101, and particularly a method for treating cancer (such as a hematologic cancer and a solid tumor, where the hematologic cancer is, for example, selected from the group consisting of leukemia, polycythemia vera, lymphoma, multiple myeloma, Waldenstrom macroglobulinemia, or heavy chain diseases; and the solid tumor is, for example, selected from the group consisting of lung cancer such as NSCLC, melanoma, ovarian cancer, pancreatic ductal adenocarcinoma, liver cancer, colorectal cancer, glioblastoma, or prostate cancer), an inflammatory or infectious disease (such as ALI, IBD, or HIV infection), an autoimmune disease, or an endocrine disease (such as RA or T1D). The present disclosure also provides an antibody with high affinity for CD101 or an antigen-binding fragment thereof, which can be used for treating a disease associated with expression and/or activity of CD101, such as cancer, an inflammatory or infectious disease, an autoimmune disease, or an endocrine disease.

Any aspect or embodiment described herein may be combined with any other aspect or embodiment disclosed herein. Although the present disclosure has been described in conjunction with the detailed description of the present disclosure, the above description is intended to illustrate rather than limit the scope of the present disclosure. The scope of the present disclosure is defined by the scope of the appended claims. Other aspects, advantages, and modifications are also within the scope of the appended claims. The patents and scientific literature mentioned herein provide the knowledge accessible to those skilled in the art. All patents and published or unpublished patent applications cited herein are incorporated herein by reference. All other published references, dictionaries, documents, manuscripts, and scientific literature cited herein are hereby incorporated herein by reference. From the following detailed description including embodiments and claims, other features and advantages of the present disclosure will be apparent.

In order to make it easy to understand the present disclosure, some terms are first defined below. Additional definitions of the following terms and other terms are set forth throughout the specification.

Unless otherwise defined, all technical and scientific terms used herein have the same meanings as commonly understood by those of ordinary skill in the art.

It should be noted that, as used herein and in the appended claims, the singular forms “a”, “an”, and “the” include plural references, unless otherwise specified clearly in the context. Thus, the terms “a”, “an”, “a or a plurality of”, and “at least one” can be used interchangeably.

When the terms “include”, “comprise”, and “have” are used herein and in the appended claims, other elements are not excluded, and these terms can be used interchangeably. For the purpose of the present application, the term “composed of . . . ” is considered as a preferred embodiment of the term “include”.

The present disclosure provides a method for treating a disease in a subject, including: an inhibitor for inhibiting expression and/or activity of CD101 is administered to the subject, where the disease is selected from the group consisting of cancer, an inflammatory or infectious disease, an autoimmune disease, or an endocrine disease.

In some embodiments of the method in the present disclosure, the cancer is selected from the group consisting of a hematologic cancer and a solid tumor. Optionally, the hematologic cancer is selected from the group consisting of leukemia, polycythemia vera, lymphoma, multiple myeloma, Waldenstrom macroglobulinemia, or heavy chain diseases. Optionally, the solid tumor is selected from the group consisting of lung cancer, melanoma, ovarian cancer, pancreatic ductal adenocarcinoma, liver cancer, colorectal cancer, glioblastoma, or prostate cancer. In some embodiments, the cancer is NSCLC. In some embodiments, the subject does not respond to treatment with a PD-1 axis inhibitor.

As used herein, the term “treatment” refers to a method for acquiring a beneficial or desired outcome (including a clinical outcome). For the purpose of the present application, a beneficial or desired clinical outcome includes, but is not limited to, one or more of the following: alleviating one or more symptoms caused by a disease, reducing the severity of a disease, stabilizing a disease (for example, preventing or delaying the exacerbation of a disease), preventing or delaying the spread of a disease, preventing or delaying the recurrence of a disease, delaying or slowing the progression of a disease, improving a state of a disease, providing the remission (partial or complete) of a disease, reducing a dose for one or more other drugs required to treat a disease, delaying the progression of a disease, improving a quality of life, increasing a body weight, and/or prolonging a survival period. The method of the present application conceives any one or more of these aspects for the treatment.

In some embodiments, the treatment can be given after one or more symptoms have already appeared. In other embodiments, the treatment can be given in the absence of symptoms. For example, the treatment may be given to a susceptible individual before symptom onset, or may be given in combination with another therapeutic agent (for example, based on a symptom history, based on genetic or other predisposing factors, a disease therapy, or any combination thereof). The treatment can also be continued after symptoms have disappeared, for example, to prevent or delay the recurrence of the symptoms.

In some embodiments of the method in the present disclosure, the inflammatory or infectious disease is selected from the group consisting of ALI, IBD, and HIV infection.

In some embodiments of the method in the present disclosure, the autoimmune disease or the endocrine disease is selected from the group consisting of RA and T1D.

In some embodiments, the disease can be cancer (such as a hematologic cancer and a solid tumor, where optionally, the hematologic cancer is selected from the group consisting of leukemia (such as acute leukemia (acute lymphocytic leukemia, acute myeloid leukemia (including myeloblastic leukemia, promyelocytic leukemia, myelomonocytic leukemia, monocytic leukemia, and erythroleukemia)) and chronic leukemia (chronic myeloid leukemia and chronic lymphocytic leukemia)), polycythemia vera, lymphoma (such as Hodgkin's lymphoma and non-Hodgkin's lymphoma), multiple myeloma, Waldenstrom macroglobulinemia, or heavy chain diseases; and optionally, the solid tumor is selected from the group consisting of sarcomas and carcinomas, such as fibrosarcoma, myxosarcoma, liposarcoma, chondrosarcoma, osteosarcoma, chordoma, angiosarcoma, endotheliosarcoma, lymphangiosarcoma, lymphangioleiomyomatosis (LAM), synovialoma, mesothelioma, Ewing sarcoma, leiomyosarcoma, rhabdomyosarcoma, colon cancer, pancreatic cancer, breast cancer, ovarian cancer, prostate cancer, squamous cell carcinoma, basal cell carcinoma, adenocarcinoma, syringocarcinoma, sebaceous carcinoma, papillary carcinoma, papillary adenocarcinoma, cystadenocarcinoma, medullary carcinoma, bronchial cancer, renal cell carcinoma, liver cancer, cholangiocarcinoma, choriocarcinoma, seminoma, embryonal carcinoma, Wilms tumor, cervical cancer, testicular tumor, lung cancer (such as NSCLC and small cell lung cancer (SCLC)), bladder cancer, epithelioma, glioma, astrocytoma, medulloblastoma, craniopharyngioma, ependymoma, pinealoma, hemangioblastoma, acoustic neuroma, oligodendroglioma, cerebral angioma, melanoma, neuroblastoma, and retinoblastoma), an inflammatory or infectious disease (such as ALI, IBD, or HIV infection), and/or an autoimmune disease or endocrine disease (such as RA or T1D).

In some embodiments of the above aspect, the inhibitor for inhibiting expression and/or activity of CD101 is an antibody specifically binding to CD101 or an antigen-binding fragment thereof.

In some embodiments, the antibody specifically binding to CD101 or the antigen-binding fragment thereof includes VH and VL. In another aspect, the present disclosure provides an antibody specifically binding to CD101 or an antigen-binding fragment thereof that includes VH and VL, where
(i) the VH includes CDR-H1, CDR-H2, and CDR-H3 having amino acid sequences set forth in SEQ ID NOs: 1-3, respectively, and the VL includes CDR-L1, CDR-L2, and CDR-L3 having an amino acid sequence set forth in SEQ ID NO: 5, an amino acid sequence QMS, and an amino acid sequence set forth in SEQ ID NO: 6, respectively; or
(ii) the VH includes CDR-H1, CDR-H2, and CDR-H3 having amino acid sequences set forth in SEQ ID NOs: 8-10, respectively, and the VL includes CDR-L1, CDR-L2, and CDR-L3 having an amino acid sequence set forth in SEQ ID NO: 12, an amino acid sequence AAT, and an amino acid sequence set forth in SEQ ID NO: 13, respectively.

As used in the present disclosure, the term “antibody” refers to an immunoglobulin molecule with an ability of specifically binding to a specific antigen. An antibody typically includes a variable region and a constant region in each heavy chain and each light chain. Variable regions of heavy and light chains of an antibody each include a binding domain interacting with an antigen. Constant regions of an antibody can mediate the binding of an immunoglobulin to a host tissue or factor. The host tissue or factor includes various cells (such as effector cells) of an immune system and components of a complement system. Thus, most antibodies have VH and VL that together constitute an antibody portion to bind to an antigen.

VH or VL includes framework regions (FRs) separated by three complementarity determining regions (CDRs). FR is generated to adjust CDR for specifically binding to an epitope. CDRs include amino acid residues in an antibody that are primarily responsible for antigen binding. From an amino terminus to a carboxyl terminus, VH and VL domains each include the following FRs and CDRs: FR1, CDR1, FR2, CDR2, FR3, CDR3, and FR4. CDR1, CDR2, and CDR3 of a VH domain are also referred to as CDR-H1, CDR-H2, and CDR-H3 herein, respectively. CDR1, CDR2, and CDR3 of a VL domain are also referred to as CDR-L1, CDR-L2, and CDR-L3 herein, respectively.

The amino acid assignment for each VH and VL domain is based on any conventional definition of CDR. The conventional definition includes a Kabat definition (Kabat, Sequences of Proteins of Immunological Interest (National Institutes of Health, Bethesda, MD, 1987 and 1991)), a Chothia definition (Chothia & Lesk, J. Mol. Biol. 196:901-917, 1987; Chothia et al., Nature 342:878-883, 1989), a combination of Chothia and Kabat definitions, an AbM definition adopted by antibody modeling software of Oxford Molecular, and a CONTACT definition of Martin et al. (www.bioinfo.org.uk/abs). The Kabat provides a widely used numbering convention (Kabat numbering system), where corresponding residues of different heavy chains or different light chains are given same numbers. The present disclosure may adopt CDRs defined by any of these numbering systems. In some embodiments, CDRs defined by Kabat are adopted. In some embodiments, CDRs defined by Chothia are adopted. In some embodiments, CDRs defined by a combination of Chothia and Kabat are adopted. In some embodiments, CDRs defined by AbM are adopted. In some embodiments, CDRs defined by CONTACT are adopted.

As used in the present disclosure, the term “binding” or “specifically binding” refers to a non-random binding reaction between two molecules, such as a non-random binding reaction between an antibody and a target antigen thereof. The binding specificity of an antibody can be determined based on affinity and/or binding activity. The affinity is usually expressed by an equilibrium constant (KD) for the dissociation of an antigen from an antibody, and is a measure of a binding strength between an epitope and an antigen-binding site of an antibody. The smaller the KD value, the stronger the binding strength between an epitope and an antibody. Alternatively, the affinity can also be expressed as an affinity constant (KA), which is 1/KD.

In some embodiments,
(i) the VH includes an amino acid sequence having at least 80%, at least 85%, at least 90%, at least 95%, at least 98%, at least 99%, or 100% sequence identity to SEQ ID NO: 4, and the VL includes an amino acid sequence having at least 80%, at least 85%, at least 90%, at least 95%, at least 98%, at least 99%, or 100% sequence identity to SEQ ID NO: 7; or
(ii) the VH includes an amino acid sequence having at least 80%, at least 85%, at least 90%, at least 95%, at least 98%, at least 99%, or 100% sequence identity to SEQ ID NO: 11, and the VL includes an amino acid sequence having at least 80%, at least 85%, at least 90%, at least 95%, at least 98%, at least 99%, or 100% sequence identity to SEQ ID NO: 14.

In some embodiments,
(i) the VH includes an amino acid sequence set forth in SEQ ID NO: 4, and the VL includes an amino acid sequence set forth in SEQ ID NO: 7; or
(ii) the VH includes an amino acid sequence set forth in SEQ ID NO: 11, and the VL includes an amino acid sequence set forth in SEQ ID NO: 14.

In some embodiments, the VH includes an amino acid sequence having at least 80%, at least 85%, at least 90%, at least 95%, at least 98%, at least 99%, or 100% sequence identity to an amino acid sequence set forth in any one of SEQ ID NOs: 15-18, and the VL includes an amino acid sequence having at least 80%, at least 85%, at least 90%, at least 95%, at least 98%, at least 99%, or 100% sequence identity to an amino acid sequence set forth in any one of SEQ ID NOs: 19-21.

In some embodiments, the VH includes an amino acid sequence set forth in any one of SEQ ID NOs: 15-18, and the VL includes an amino acid sequence set forth in any one of SEQ ID NOs: 19-21.

In a preferred embodiment,
(i) the VH includes an amino acid sequence set forth in SEQ ID NO: 15, and the VL includes an amino acid sequence set forth in SEQ ID NO: 19; or
(ii) the VH includes an amino acid sequence set forth in SEQ ID NO: 15, and the VL includes an amino acid sequence set forth in SEQ ID NO: 20; or
(iii) the VH includes an amino acid sequence set forth in SEQ ID NO: 17, and the VL includes an amino acid sequence set forth in SEQ ID NO: 19; or
(iv) the VH includes an amino acid sequence set forth in SEQ ID NO: 17, and the VL includes an amino acid sequence set forth in SEQ ID NO: 20.

As used herein, the “sequence identity” refers to an identity degree between any given query sequence and a subject sequence. Those skilled in the art will easily understand how to determine an identity between two peptides (such as an unmodified peptide and a peptide variant. For example, an identity between two sequences can be calculated after the two sequences are aligned to reach the highest identity level, for example, gaps can be introduced. An identity can also be calculated by the published algorithms. Non-restrictive examples of such mathematical algorithms include an algorithm in Myers and Miller (1988) CABIOS 4:11-17, a local homology algorithm in Smith et al. (1981) Adv. Appl. Math. 2:482, a homology alignment algorithm in Needleman and Wunsch (1970) J. Mol. Biol. 48:443-453, modified forms of a method of searching for a homology in Pearson and Lipman (1988) Proc. Natl. Acad. Sci. 85:2444-2448 and an algorithm in Karlin and Altschul (1990) Proc. Natl. Acad. Sci. USA 87:2264, and an algorithm recorded in Karlin and Altschul (1993) Proc. Natl. Acad. Sci. USA 90:5873-5877. The sequence comparison (namely, alignment) for determining a sequence identity can be implemented with a program based on such a mathematical algorithm. The program can be properly executed by a computer. Examples of such a program include, but are not limited to, CLUSTAL and ALIGN programs (version 2.0) of a PC/Gene s package and GAP, BESTFIT, BLAST, FASTA, and TFASTA of a Wisconsin genetics software package. For example, these programs can be implemented with initial parameters to conduct an alignment.

In addition, when a sequence identity between two amino acid sequences is determined, technicians may consider the so-called conservative amino acid substitution. The conservative amino acid substitution can often be described as an amino acid substitution in which an amino acid residue is substituted with another amino acid residue having a similar chemical structure, which has little or no impact on a function, activity, or other biological properties of a polypeptide. Such a conservative amino acid substitution is well known in the art.

The conservative substitution preferably refers to a substitution in which an amino acid residue from the following groups (a) to (e) is substituted with another amino acid residue from the same group as the amino acid residue: (a) small aliphatic, non-polar, or weakly polar residues: Ala, Ser, Thr, Pro, and Gly; (b) polar and negatively charged residues and (uncharged) amides thereof: Asp, Asn, Glu, and Gln; (c) polar and positively charged residues: His, Arg, and Lys; (d) large aliphatic and non-polar residues: Met, Leu, He, Val, and Cys; and (e) aromatic residues: Phe, Tyr, and Trp.

Particularly preferred conservative substitutions are as follows: Ala to Gly or Ser; Arg to Lys; Asn to Gln or His; Asp to Glu; Cys to Ser; Gln to Asn; Glu to Asp; Gly to Ala or Pro; His to Asn or Gln; Ile to Leu or Val; Leu to Ile or Val; Lys to Arg, Gln, or Glu; Met to Leu, Tyr, or Ile; Phe to Met, Leu, or Tyr; Ser to Thr; Thr to Ser; Trp to Tyr; Tyr to Trp; and/or Phe to Val, Ile, or Leu.

In some embodiments, the antibody and the antigen-binding fragment thereof in the present disclosure include variants, and the variants may include one or more amino acid modifications. A single variant can include a substitution, a deletion, an insertion, an addition, or any combination thereof, as long as the variant can specifically bind to an antigen. Methods and techniques for preparing the variants are well known to those skilled in the art.

In some embodiments, the antibody of the present disclosure includes a Fc region of an immunoglobulin or a portion thereof. For example, in some embodiments, the antibody of the present disclosure includes a CH2 and/or CH3 region. In some embodiments, the antibody includes a hinge region, a CH2 region, and a CH3 region.

In some embodiments, the antibody can be selected from the group consisting of IgG, IgA, IgM, IgE, and IgD isotypes, such as IgG1, IgG2, IgG3, and IgG4 subtypes. In some embodiments, the antibody can be selected from the group consisting of a murine antibody, a chimeric antibody, a humanized antibody, and a fully human antibody. In some embodiments, the antigen-binding fragment can be selected from the group consisting of Fab, Fab′, F(ab′) 2, Fv, scFv, and ds-scFv. In some embodiments, the antibody can be a bispecific antibody or a multispecific antibody.

The term “antibody” used in the present disclosure should be understood in its broadest sense, and includes a monoclonal antibody (including a full-length monoclonal antibody), a polyclonal antibody, an antibody fragment, and a multispecific antibody including at least two antigen-binding regions (such as a bispecific antibody). The antibody may include an additional modification, such as a non-natural amino acid, a mutation in a Fc region, and a mutation at a glycosylation site. The antibody also includes an antibody with a post-translational modification (PTM), a fusion protein including an antigenic determinant of an antibody, and an immunoglobulin molecule including any other modification for an antigen recognition site, as long as these antibodies exhibit an expected biological activity.

In some embodiments, the antibody includes a chimeric antibody, such as a humanized form of a murine antibody. Such a humanized antibody can be prepared by a known technique, and exhibits the advantage of reducing the immunogenicity when administered to the human. In an embodiment, the humanized antibody includes a variable region of a murine antibody (or only an antigen-binding site thereof) and a constant region derived from a human antibody. Alternatively, a humanized antibody fragment can include an antigen-binding site of a murine antibody and a variable region fragment derived from a human antibody (lacking an antigen-binding site). Processes for producing chimeric antibodies and further engineered antibodies include those processes described in the following: Riechmann et al. (Nature 332:323, 1988); Liu et al. (PNAS 84:3439, 1987); Larrick et al. (Bio/Technology 7:934, 1989); and Winter and Harris (TIPS 14:139, May 1993).

Antibodies (such as chimeric and humanized antibodies) that can be produced through genetic engineering each include both human and non-human moieties, and can be manufactured by standard recombinant DNA techniques. Such chimeric and humanized antibodies can be produced through genetic engineering by standard DNA techniques known in the art.

As used in the present disclosure, the term “antigen-binding fragment” of an antibody refers to one or more antibody fragments that maintain the ability to specifically bind to an antigen (such as CD101). It has been proved that an antigen-binding function of an antibody can be implemented by a fragment of the full-length antibody. Examples of the antigen-binding fragment include: (i) a Fab fragment, which is a monovalent fragment including VH, VL, CH1, and CL domains; (ii) a F(ab′)₂ fragment, which is a bivalent fragment including two Fab fragments linked through a disulfide bond in a hinge region; (iii) a Fab′ fragment, which is essentially Fab but has a partial hinge region (as shown in FUNDAMENTAL IMMUNOLOGY (Paul ed., 3.sup.rd ed. 1993)); (iv) a Fd fragment, which includes VH and CH1 domains; (v) a Fd′ fragment, which includes VH and CH1 domains and one or more cysteine residues located at a C-terminus of a CH1 domain; (vi) a Fv fragment, which includes VH and VL domains of a single arm of an antibody; (vii) a dAb fragment (Ward et al. (1989) Nature 341:544-546), which includes a VH domain; (viii) a separate CDR; and (ix) a nanobody, which is VH including a single variable domain and two constant domains. In addition, although the two domains of the Fv fragment, VH and VL, are encoded by separate genes, the two domains can be linked with a synthetic linker by a recombination method. The synthetic linker enables the generation of a single protein chain in which the VH and VL are paired to produce a monovalent molecule (known as single-chain Fv (ScFv), see, for example, Bird et al. (1988) Science 242, 423-426 and Huston et al. (1988) Proc. Natl. Acad. Sci. USA 85, 5879-5883). Such a single-chain antibody is also intended to be covered by the term “antigen-binding fragment” of an antibody. In addition, the term includes a linear antibody with a pair of tandem Fd fragments (VH-CH1-VH-CH1), which forms an antigen-binding region together with a complementary light chain polypeptide and a modified version of any of the above fragments with an antigen-binding activity retained.

These antigen-binding fragments can be prepared by the conventional techniques known to those skilled in the art, and the utility of the fragments is screened in the same way as intact antibodies.

In an aspect, the present disclosure provides a nucleic acid including a nucleotide sequence encoding the antibody or the antigen-binding fragment thereof of the present disclosure.

The terms “nucleic acid”, “nucleotide”, and “nucleotide sequence” can be used interchangeably, and refer to oligomers and polymers of any length that are essentially made up of nucleotides (such as deoxyribonucleotides and/or ribonucleotides). The nucleic acid can include purine and/or pyrimidine bases and/or other natural bases (such as xanthine, inosine, and hypoxanthine), chemically or biochemically modified (such as methylated) bases, and unnatural or derived nucleotide bases. A backbone of the nucleic acid can include a sugar and a phosphate group normally present in RNA or DNA, and/or one or more modified or substituted sugars and/or one or more modified or substituted phosphate groups. A modification for a phosphate group or a sugar can be introduced to improve the stability, the resistance to enzymatic degradation, or some other useful properties. The nucleic acid can be, for example, double-stranded, partially double-stranded, or single-stranded. When the nucleic acid is single-stranded, the nucleic acid can have a sense strand or an antisense strand. The nucleic acid can be circular or linear. As used herein, the term “nucleic acid” encompasses both DNA and RNA, including a genome, pre-mRNA, mRNA, cDNA, and a recombinant or synthetic nucleic acid carried by a vector. For the purpose of the use described herein, it should be understood that a polynucleotide can be modified by any method available in the art.

Those skilled in the art will understand that many different polynucleotides and nucleic acids can encode a same polypeptide due to the degeneracy in the genetic code. In addition, it should be understood that a nucleotide substitution not affecting a polypeptide sequence encoded by the polynucleotide described herein may be conducted by technicians using a conventional technique to reflect the codon use of any specific host organism in which the polypeptide is to be expressed.

In another aspect, the present disclosure provides a vector including the nucleic acid of the present disclosure.

The term “vector” used herein refers to a nucleic acid delivery vehicle into which a nucleotide can be inserted. When a vector can make a protein encoded by an inserted nucleotide expressed, the vector is called an expression vector. A vector can be introduced into a host cell through a method such as transformation, transduction, or transfection, such that a genetic material element carried by the vector can be expressed in the host cell. Vectors are well known to those skilled in the art, and include but are not limited to: (1) a plasmid; (2) a phagemid; (3) a cosmid; (4) an artificial chromosome, such as a yeast artificial chromosome, a bacterial artificial chromosome, or a P1-derived artificial chromosome; (5) a phage, such as a 2 phage or an M13 phage; and (6) an animal virus, such as a retrovirus, an adenovirus, an adeno-associated virus, a lentivirus, a herpes virus, a poxvirus, and a baculovirus. A vector may include a plurality of elements to control the expression, including but not limited to a promoter sequence, a transcription initiation sequence, an enhancer sequence, a selection element, and a reporter gene. In addition, a vector can include a replication origin.

A recombinant expression vector can be any suitable recombinant expression vector. Suitable vectors include vectors designed for propagation and amplification or for expression or for both, such as plasmids and viruses. For example, vectors can be selected from the pUC series (Fermentas Life Sciences, Glen Burnie, Md.), the pBluescript series (Stratagene, LaJolla, CA), the pET series (Novagen, Madison, Wis.), the pGEX series (Pharmacia Biotech, Uppsala, Sweden), and the pEX series (Clontech, Palo Alto, Calif.). Phage vectors such as AGT10, AGT11, AZapII (Stratagene), AEMBL4, and ANM1149 can also be adopted. Examples of plant expression vectors that can be used in the present disclosure include pBI01, pBI101.2, pBI101.3, pBI121, and pBIN19 (Clontech). Examples of animal expression vectors that can be used in the present disclosure include pcDNA, pEUK-Cl, pMAM, and pMAMneo (Clontech).

In another aspect, the present disclosure provides a host cell including the nucleic acid of the present disclosure or the vector of the present disclosure.

As used herein, the term “host cell” refers to a cell in which an expression vector has been introduced. Any cell can be adopted as a host cell for the nucleic acid or vector in the present disclosure. In some embodiments, the host cell can be a prokaryotic cell, a fungal cell, a yeast cell, or a higher eukaryotic cell such as a mammalian cell. Suitable prokaryotic cells include, but are not limited to, eubacteria, such as gram-negative or gram-positive organisms, such as Enterobacteriaceae, such as Escherichia, such as Escherichia coli (E. coli); Enterobacter; Erwinia; Klebsiella; Proteus; Salmonella, such as Salmonella typhimurium; Serratia, such as Serratia marcescens; Shigella; Bacillus, such as Bacillus subtilis (B. subtilis) and Bacillus licheniformis (B. licheniformis); Pseudomonas, such as Pseudomonas aeruginosa (P. aeruginosa); and Streptomyces. In some embodiments, the host cell is a human cell. In some embodiments, the host cell is an immune cell. In some embodiments, the host cell includes, for example, a CHO cell, such as a CHO-S cell and a CHO-K1 cell, or an HEK293 cell, such as HEK293A, HEK293T, and HEK293FS.

In yet another aspect, the present disclosure provides a pharmaceutical composition, including: the antibody or the antigen-binding fragment thereof of the present disclosure, and a pharmaceutically acceptable carrier or excipient.

The term “pharmaceutically acceptable” means that the carrier or adjuvant is compatible with other components of the composition and is not substantially toxic to a recipient, and/or the carrier or adjuvant is approved or can be used for inclusion in the pharmaceutical composition administered parenterally to the human.

In some embodiments, the carrier or excipient used with the composition of the present disclosure includes, but is not limited to, maleic acid, tartaric acid, lactic acid, citric acid, acetic acid, sodium bicarbonate, sodium phosphate, histidine, glycine, sodium chloride, potassium chloride, calcium chloride, zinc chloride, water, dextrose, N-methylpyrrolidone, dimethyl sulfoxide, N,N-dimethylacetamide, ethanol, propylene glycol, polyethylene glycol, diethylene glycol monoethyl ether, and a surfactant polyoxyethylene-sorbitan monooleate.

In some embodiments of the above aspects, the method of the present disclosure further includes: a second therapeutic agent is administered to the subject, or the inhibitor for inhibiting expression and/or activity of CD101 is further used in combination with the second therapeutic agent, or the drug or the pharmaceutical composition of the present disclosure further includes the second therapeutic agent. The second therapeutic agent is selected from the group consisting of an antibody, a chemotherapeutic agent, and a small molecule drug.

As used herein, the term “administration” refers to the introduction of an antibody or a composition into a subject, and includes the parallel or sequential introduction of antibodies or compositions. The term “administration” can refer to, for example, treatment, pharmacokinetics, diagnosis, research, placebos, and experimental methods. The term “administration” also covers in vitro and ex vivo treatment. A composition or an agent can be introduced into a subject through any suitable route. Examples of an appropriate method for administering the antibody or composition of the present disclosure include oral administration, intradermal injection, subcutaneous injection, intramuscular injection, intraosseous injection, intraperitoneal injection, and intravenous injection, and systemic or topical administration to a vicinity of a target site, but are not limited thereto. The administration includes both administration by oneself and administration by others. The administration can be conducted through any suitable route. The appropriate administration route allows a composition or an agent to exert an expected function. For example, if the appropriate route is intravenous injection, the antibody or the composition is administered by introducing the antibody or the composition into a vein of the subject.

In some embodiments of the present disclosure, the antibody or the composition of the present disclosure is packaged with or stored in a device for administration. A device for an injectable preparation includes, but is not limited to, an injection port, an automatic injector, an injection pump, and an injection pen. A device for a nebulized or powdered preparation includes, but is not limited to, an inhaler, an insufflator, and an aspirator. Therefore, the present disclosure includes a use of an administration device carrying the antibody or composition of the present disclosure in treatment or prevention of one or more of the conditions described herein.

The subject expected to be administered includes, but is not limited to, a human (namely, a male or female of any age group, such as a pediatric subject (such as an infant, a child, and an adolescent) or an adult subject (such as a youth, a middle-aged person, or the elderly)) and/or another non-human animal, such as a mammal (such as a primate).

In some embodiments, the second therapeutic agent can be selected from the group consisting of an antibiotic derivative, such as actinomycin D, daunorubicin, doxorubicin, idarubicin, anthracycline, mitoxantrone, bleomycin, plicamycin (mithramycin), mitomycin, and dactinomycin; a platinum complex, such as cisplatin, oxaliplatin, carboplatin, procarbazine, hydroxyurea, mitotane, and amino-glutamine; paclitaxel, such as paclitaxel and docetaxel; camptothecin, including synthetic topotecan; an antimetabolite, such as fluorouracil, 5-fluorouracil, floxuridine, capecitabine, cytarabine, interferon α-2b, glutamic acid, mithramycin, mercaptopurine, and 6-thioguanine; a folic acid analogue, such as demethotrexate, methotrexate, pterin, and trimethotrexate; a purine analogue, such as fludarabine, 6-mercaptopurine, sulfadiazine, and thioguanine; a pyrimidine analogue, such as ancitabine, azacitidine, 6-azauridine, carmofur, cytarabine, dideoxyuridine, doxifluridine, enocitabine, and floxuridine; an antiproliferative/antimitotic agent, such as a natural product such as vinca alkaloid (vinblastine and vincristine), nocodazole, epothilone, vinorelbine, epipodophyllotoxin (etoposide and teniposide), nitrogen mustard cyclophosphamide and an analogue thereof (melphalan, chlorambucil, hexamethylmelamine, and thiotepa), a nitrosourea alkylating agent (carmustine) and an analogue thereof, streptozotocin, and triazene (dacarbazine); an alkylating agent, such as thiotepa and cyclophosphamide; a DNA damaging agent, such as dactinomycin, amsacrine, busulfan, carboplatin, chlorambucil, ifosfamide, melphalan, chloroethylamine, mitomycin, mitoxantrone, nitrosourea, procarbazine, taxotere, teniposide, etoposide, and triethylenethiophosphoramide; an antiestrogen, such as tamoxifen, raloxifene, droloxifene, 4-hydroxytamoxifen, trioxifene, keoxifene, LY117018, onapristone, and toremifene; an antiandrogen, such as flutamide, nilutamide, bicalutamide, leuprolide, and goserelin; an aromatase inhibitor, such as aminoglutathione, megestrol acetate, exemestane, formestane, fadrozole, vorozole, letrozole, and anastrozole; a hormone, such as medroxyprogesterone, estramustine phosphate sodium, ethinyl estradiol, oestradiol, megestrol acetate, methyltestosterone, diethylstilbestrol diphosphate, chlorotrianisene, and testolactone; a corticosteroid, such as cortisone, dexamethasone, hydrocortisone, methylprednisolone, prednisone, and prednisolone; a nitrogen mustard derivative, such as melphalan, chlorambucil, chlormethine, and thiotepa; a steroid and a combination thereof, such as betamethasone sodium phosphate; an antiplatelet drug; an antimetastatic agent; an antisecretory agent (breveldin); an antiadrenergic agent, such as amino-glutamine, mitotane, and trilostane; a growth factor inhibitor, a fibroblast growth factor inhibitor, an angiotensin receptor blocker, and a nitric oxide donor; a cell cycle inhibitor, a topoisomerase inhibitor (doxorubicin, daunorubicin, actinomycin D, teniposide, epirubicin, etoposide, idarubicin, irinotecan, mitoxantrone, topotecan, and irinotecan); a growth factor signaling kinase inhibitor; a dysfunction inducer; a toxin, such as cholera toxin, ricin toxin, Pseudomonas exotoxin, Bordetella pertussis adenylate cyclase toxin, diphtheria toxin, and caspase activator; a fibrinolytic agent, such as a tissue plasminogen activator, streptokinase, urokinase, aspirin, dipyridamole, ticlopidine, and clopidogrel; an immunosuppressant, such as tacrolimus, sirolimus, azathioprine, and mycophenolate mofetil; cycomelamine and melamine, including A'er Weitaming, trimethylene melamine, triethylenephosphoramide, triethylenethiophosphoramide, and trimethylene melamine; bryostatin; kallistatin; an anti-angiogenic agent, such as retinoid and a derivative thereof, 2-methoxyestradiol, suramin, and squalamine; a tissue inhibitor of metalloprotease-1, a tissue inhibitor of metalloprotease-2, a plasminogen activator inhibitor-1, a plasminogen activator inhibitor-2, a cartilage-derived inhibitor, a platelet factor 4, protamine sulfate (clupeine), a sulfated chitin derivative (prepared from a hairy crab shell), a sulfated polysaccharide-peptidoglycan complex (sp-pg), staurosporine, and a matrix metabolism regulator, including a proline analogue (l-azetidine-2-carboxylic acid (LACA)), cis-hydroxyproline, d,I-3,4-dehydroproline, thioproline, a,o′-bipyridine, β-aminopropionitrile fumarate, 4-propyl-5-(4-pyridyl)-2 (3H)-oxazolone, mitoxantrone, heparin, interferon, a chymotrypsin inhibitor, β-cyclodextrin, a tetradecyl sulfate, epothilone, fumagillin, gold sodium thiomalate, d-penicillamine, a β1-anticollagenase α2-antiplasmin, bisantrene, lobenzarit disodium, N-2-carboxyphenyl-4-chloroanthranilic acid disodium salt (CCA), thalidomide, a vasoinhibitory steroid, carboxyaminoimidazole, and a metalloproteinase inhibitor such as BB-94; other anti-angiogenic agents, including antibodies and preferably monoclonal antibodies against the following angiogenic growth factors: β-FGF, α-FGF, FGF-5, VEGF isotypes, VEGF-C, HGF/SF, and Ang-1/Ang-2; a PI3K inhibitor; a PI3Ky inhibitor; a PI3Kp inhibitor; a PI3Ka inhibitor; a pan-PI3K inhibitor; an SYK inhibitor; inhibitors for the following enzymes or proteins: Abl, activated CDC kinase (ACK), adenosine A2B receptor (A2B), apoptosis signal-regulating kinase (ASK), Auroa kinase, Bruton's tyrosine kinase (BTK), bromodomain and extratermina (BET) bromodomain (BRD), c-Kit, c-Met, CDK-activating kinase (CAK), calmodulin-dependent protein kinase (CaMK), cyclin-dependent kinase (CDK), casein kinase (CK), discoidin domain receptor (DDR), epidermal growth factor receptor (EGFR), focal adhesion kinase (FAK), Flt-3, FYN, glycogen synthase kinase (GSK), HCK, histone deacetylase (HDAC), IKK such as IKKBe, isocitrate dehydrogenase (IDH), Janus kinase (JAK), KDR, lymphocyte-specific protein tyrosine kinase (LCK), lysyl oxidase protein, lysyl oxidase-like protein (LOXL), LYN, matrix metalloproteinase (MMP), MEK, mitogen-activated protein kinase (MAPK), NEK9, NPM-ALK, p38 kinase, platelet-derived growth factor (PDGF), phosphorylase kinase (PK), polo-like kinase (PLK), phosphatidylinositol 3-kinase (PI3K), protein kinase (PK), PYK, spleen tyrosine kinase (SYK), serine/threonine kinase TPL2, serine/threonine kinase STK, signal transducer and activator of transcription (STAT), SRC, serine/threonine protein kinase (TBK) such as TBK1, TIE, tyrosine kinase (TK), and vascular endothelial growth factor receptor (VEGFR); an antifibrotic agent; a nitrogen mustard alkylating agent; an ASK inhibitor; a BTK inhibitor; a DDR inhibitor; an HDAC inhibitor; a JAK inhibitor; an LOXL inhibitor; an LOXL2 inhibitor; an MMP inhibitor; others, such as dicarbohydrazide, asparaginase, mitotane, vincristine sulfate, vinblastine sulfate, and etoposide; edatrexate; methamphetamine; demecolcine; thiadiazinthion; metformin; elliptinium acetate; epothilone; gastrin; gallium nitrate; hydroxyurea; lentinan; folinic acid; lonidamine; maytansine, such as maytenin and ansamitocin; mitoguazone; mitoxantrone; Mopidamo; sodium nitrate; pentostatin; phenamet; pirarubicin; losoxantrone; fluoropyrimidine; podophyllic acid; 2-ethylhydrazide; procarbazine; polysaccharide-K (PSK); razoxane; rhizoxin; Xizuofeilan; spirogermanium; tenuazonic acid; triazinone; 2,2′,2″-trichlorotriphenylamine; ethyl carbamate; vindesine; dacarbazine; mannomustine; mitobronitol; mitolactol; pipobroman; hemicytosine; arabinoside (“Ara-C”); cyclophosphamide; thiopeta; chlorambucil; gemcitabine (GEMZAR®); 6-thioguanine; mercaptopurine; methotrexate; vinblastine; platinum; etoposide (VP-16); ifosfamide; Miqu anthraquinone; Wanxin base; vinorelbine; Nuowanlong; teniposide; edatrexate; daunomycin; aminopterin; Xeloda; ibandronate; CPT-11; a topoisomerase inhibitor RFS2000; difluoromethylornithine (DFMO); retinoic acid; capecitabine; FOLFIRI (fluorouracil, folinic acid, and irinotecan); other therapeutic antibodies, including rituximab, oregovomab, panitumumab, trastuzumab, simtuzumab, abagovomab, adecatumumab, afutuzumab, alemtuzumab, altumomab, amatuximab, anatumomab, arcitumomab, bavituximab, bectumomab, bevacizumab, bivatuzumab, blinatumomab, brentuximab, cantuzumab, catumaxomab, cetuximab, citatuzumab, cixutumumab, daratumumab, drozitumab, duligotumab, dusigitumab, detumomab, dacetuzumab, dalotuzumab, ecromeximab, elotuzumab, ensituximab, ertumaxomab, etaracizumab, farletuzumab, ficlatuzumab, figitumumab, flanvotumab, futuximab, ganitumab, gemtuzumab, girentuximab, glembatumumab, imga, ibritumumab, inotuzumab, intetumumab, ipilimumab, iratumumab, labetuzumab, lexatumumab, lintuzumab, lorvotuzumab, lucatumumab, mapatumumab, matuzumab, milatuzumab, minretumomab, mitumomab, moxetumomab, narnatumab, naptumomab, necitumumab, parsatuzumab, patritumab, pemtumomab, pertuzumab, pintumomab, pritumumab, racotumomab, radretumab, rilotumumab, rituximab, robatumumab, satumomab, sibrotuzumab, siltuximab, solitomab, tacatuzumab, taplitumomab, tenatumomab, teprotumumab, tigatuzumab, tositumumab, tuveltuzumab, tuveltuzumab, votumumab, zalutumumab, CC49, and 3F8; a cytokine, including, but not limited to, IL-2, IL-3, IL-4, IL-5, IL-6, IL-7, IL-10, IL-12, IL-13, IL-15, CD40, CD40L, and TNF-α; and therapeutic radiopharmaceutical radioactive isotope particles such as indium-111, yttrium-90, or iodine-131 and any combination thereof.

### EXAMPLES

The present application is further clarified through the following examples, but any or combinations of the examples should not be construed as limiting the scope or implementation of the present application. The scope of the present application is defined by the appended claims. In conjunction with the specification and the general knowledge of the art, those of ordinary skill in the art can clearly understand the scope defined by the claims. Those skilled in the art may make any modifications or changes to the technical solutions of the present application without deviating from the spirit and scope of the present application, and such modifications and changes are also included in the scope of the present application.

### Example 1 Preparation and Detection of Purified Antibodies

### 1. Preparation of CHO-S-CD101 and 293F-CD101 Expression Cell Lines

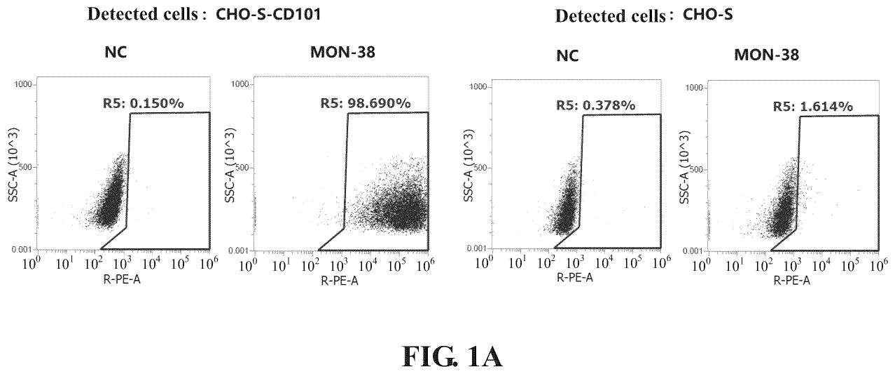

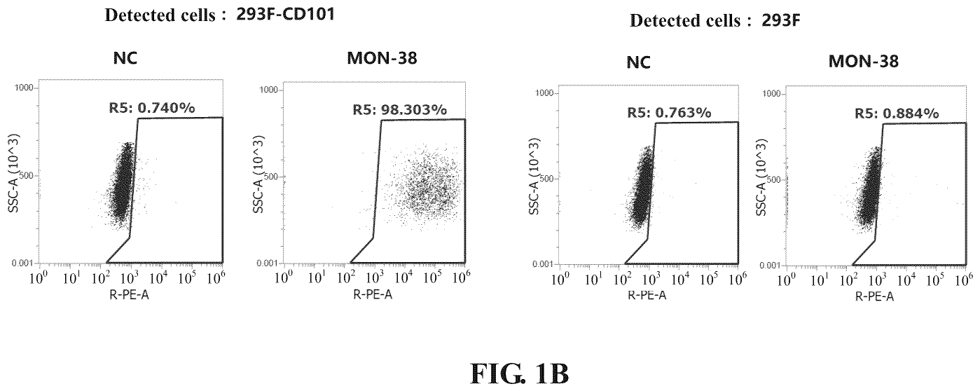

According to sequence information of CD101, a codon-optimized coding sequence for CD101 (Genbank accession number: NM_004258.6) was synthesized and subcloned into a lentiviral expression vector Lenti-CMV-puro to prepare a vector for target gene overexpression. A lentivirus was prepared with the constructed lentiviral vector for overexpression and used to infect CHO-S and 293F cells (THERMO FISHER: FreeStyle™ 293-F Cells, R79007), and stable cell lines were selected with puromycin. The prepared cell lines were incubated with an anti-CD101 monoclonal antibody (MON-38 monoclonal antibody (ANT-28), THERMO FISHER, 14-1019-82), and the expression of a target gene was detected by FACS. As shown in FIG. 1A and FIG. 1B, the CHO-S-CD101 and 293F-CD101 expression cell lines that could be used for immunization titer detection had been successfully selected.

### 2. Mouse Immunization and Detection

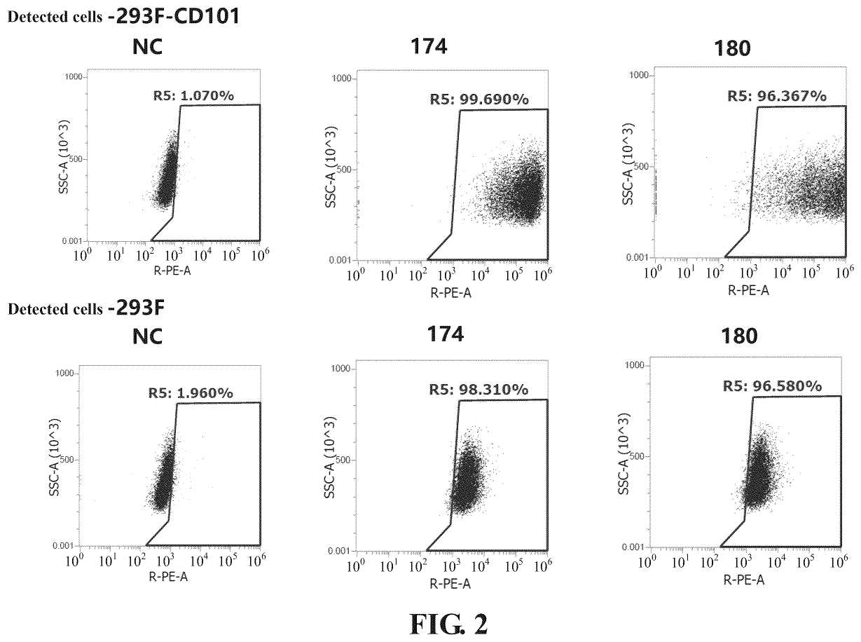

Ten 6-week-old female Balb/c mice (SPF grade, which were numbered 172 to 181) were intraperitoneally injected with CHO-S-CD101 cells for immunization. Three immunizations were conducted through intraperitoneal injection on day 0, day 49, and day 63, respectively. After the immunizations, 10 μL of orbital blood was collected from each mouse and centrifuged to produce serum, and the serum was diluted at 1:1,000 and detected by FACS for an immunization titer. According to FACS detection results (FIG. 2), mice 174 and 180 with prominent immunization titer detection results were selected for rush immunotherapy and hybridoma fusion.

### 3. Hybridoma Fusion and Screening

After undergoing 3 d of rush immunotherapy, mice were sacrificed, and mouse spleens were collected under sterile conditions to prepare single-cell suspensions of B cells. The single-cell suspensions of B cells each were mixed with non-secretory SP2/0 myeloma cells (Shanghai Cell Bank, Chinese Academy of Sciences, catalog number: TCM42) at a ratio of 1:1, and cell fusion was conducted with a BTX cell electrofusion instrument. Merged cells were cultured in a complete medium (DMEM, 20% FBS and HAT) for about 10 d, then the complete medium was replaced with an HT medium, and the merged cells were further cultured for 2 d. A resulting culture supernatant was collected for specific antibody detection.

Specifically, the collected culture supernatant was incubated with CHO-S-CD101 cells, and FACS was conducted for identification. Clones with strong binding according to FACS were selected for flow cytometry retesting, and then the clones with strong binding according to FACS were subcloned. At the end of subcloning, FACS was conducted for identification. The above process was repeated for a plurality of rounds until monoclones were produced.

FACS was conducted according to a standard protocol. In short, 293F and 293F-CD101 cells in a logarithmic growth phase each of 3×10⁵ were fully mixed with 100 μL of a hybridoma supernatant, and incubated for 1 h at room temperature. Cells were washed with PBS 3 times. A secondary antibody (PE-labeled anti-mouse IgG H&L, which was diluted 1:1,000) was added, thorough mixing was conducted, and incubation was conducted for 30 min at room temperature in the dark. Cells were washed with PBS 3 times and then resuspended with PBS. A fluorescence intensity was read by a multimode microplate reader to determine a titer of a fusion clone. In the detection, NC was PBS, and PC was an immune serum diluted at 1:1,000.

According to FACS results, the three clones 19-A11-H3-B11, 2-F12-F8-B9, and 25-D1-G2-C9 (which were re-numbered as A1 to A3, respectively) were selected for further hybridoma sequencing.

### 4. Hybridoma Sequencing

According to a standard protocol, total RNA was extracted from hybridoma cells and reverse-transcribed into a cDNA sample. VH and VL of an antibody were amplified by polymerase chain reaction (PCR) with hybridoma sequencing primers, and then TA cloning was conducted. Fragments produced by PCR were subcloned into a T vector, and clones were picked for sequencing. Hybridoma sequencing results were shown in Table 1 below, where A1 and A3 sequences were identical.

TABLE 1
CDR, VH, and VL sequences of A1 to A3 antibodies

| | | | SEQ ID |
|---|---|---|---|
| Antibody name | Region | Sequence | NO: |
| A1/A3 | CDR-H1 | GFTLTEYT | 1 |
| (19-A11-H3-B11/ | CDR-H2 | FYPGSGGI | 2 |
| 25-D1-G2-C9) | CDR-H3 | ARHAFYRNYEYFDV | 3 |
| | VH | EVQLQQSGAELVRPGASVKLSCKASGFTLT | 4 |
| | | EYTIHWVKQRSGQGLEWIGWFYPGSGGIK | |
| | | YNEKFKDKATLTADESSSTVYMELSRLTSE | |
| | | DSAVYFCARHAFYRNYEYFDVWGTGTTV | |
| | | TVSS | |
| | CDR-L1 | KSLLHSNGITY | 5 |
| | CDR-L2 | QMS | |
| | CDR-L3 | AQNLELPLT | 6 |
| | VL | DIVMTQAAFSNPVTLGTSASISCRSSKSLLH | 7 |
| | | SNGITYLYWYLQKPGQSPQLLIYQMSNLAS | |
| | | GVPDRFSSSGSGTDFTLRISRVEAEDVGVY | |
| | | YCAQNLELPLTFGAGTKLELK | |
| A2 | CDR-H1 | GYAFTHYF | 8 |
| (2-F12-F8-B9) | CDR-H2 | ITPGSGGT | 9 |
| | CDR-H3 | ARGNHGNY | 10 |
| | VH | EVQLQQSGAELVRPGTSVKVSCKASGYAF | 11 |
| | | THYFIEWVKQRPGQGLEWIGVITPGSGGTN | |
| | | YNEKFKGKATLTADKSSSTAYMQLSSLTSE | |
| | | DSAVYFCARGNHGNYWGQGTTLTVSS | |
| | CDR-L1 | ENIYSN | 12 |
| | CDR-L2 | AAT | |
| | CDR-L3 | QHFWGNPWT | 13 |
| | VL | DIQLTQPPASLSVSVGETVTITCRASENIYSN | 14 |
| | | LAWYQQKQGKSPQLLVYAATNLADGVPSR | |
| | | FSGSGSGTQYSLKINSLQSEDFGSYYCQHF | |
| | | WGNPWTFGGGTKLEIK | |

### Example 2 Preparation and Detection of Chimeric Antibodies

### 1. Construction and Verification of Expression Vectors for Chimeric Antibodies

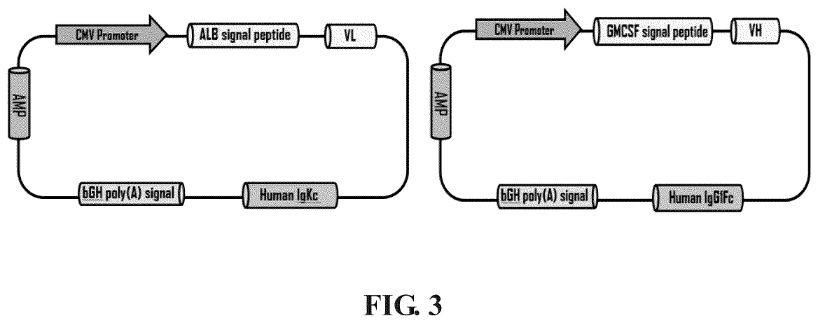

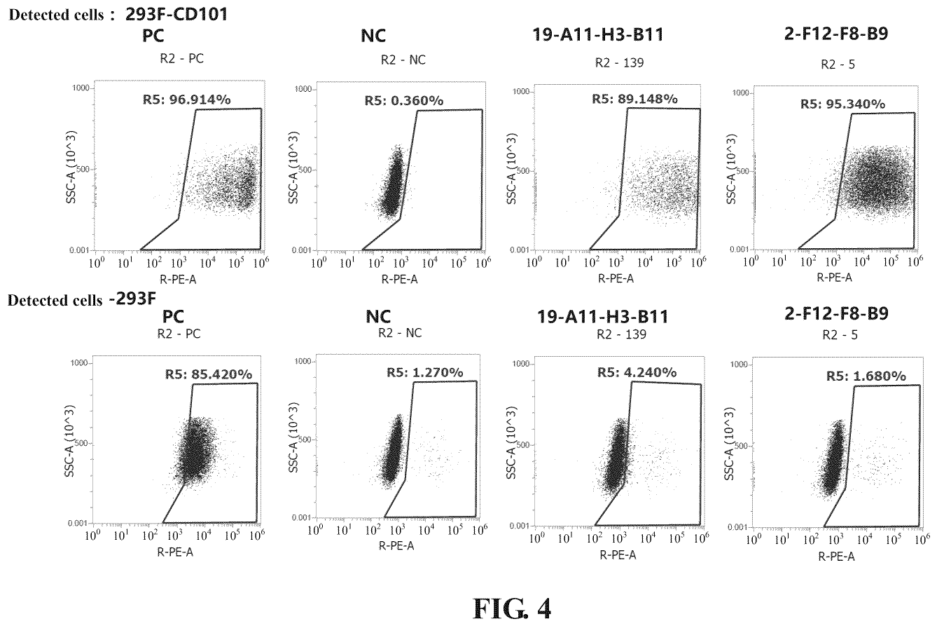

A VH coding sequence of an antibody produced from hybridoma sequencing was subcloned into a pcDNA3.4-IgG1 expression vector, and a VL coding sequence was subcloned into a pcDNA3.4-IgKc expression vector (schematic diagrams of the vectors were shown in FIG. 3). The constructed heavy-chain and light-chain expression vectors were transiently co-transfected into 293F cells (serum-free culture), and a resulting culture supernatant was collected for FACS. A method of FACS was the same as described in Example 1, where PC was a positive serum diluted at 1:1,000 (100 μL/well) and NC was PBS. Results (FIG. 4) showed that the two constructed chimeric antibodies both could specifically bind to CD101.

### 2. Expression and Purification of Chimeric Antibodies

PBS was added at 2 mL/well to a 6-well plate, then 40 μg of a pcDNA3.1-IgG1 expression vector and 80 μg of a pcDNA3.1-IgKc expression vector were added, and thorough mixing was conducted by pipetting up and down. 360 μL of a LVTransm transfection reagent was added, then thorough mixing was immediately conducted by pipetting up and down, and the plate was allowed to stand at room temperature for 10 min. A DNA/LVTransm complex was added to 100 mL of 293F cells (cell density: 1×10⁶ mL), and gentle shaking was conducted for thorough mixing. The cells were further cultured for 7 d in a 37° C. and 5% CO₂ incubator at 130 rpm. A culture supernatant was collected through centrifugation and filtered through a 0.45 μm filter membrane to produce a filtrate. The filtrate was transferred to a sterile centrifuge tube and loaded on a Protein A affinity column to purify a chimeric antibody.

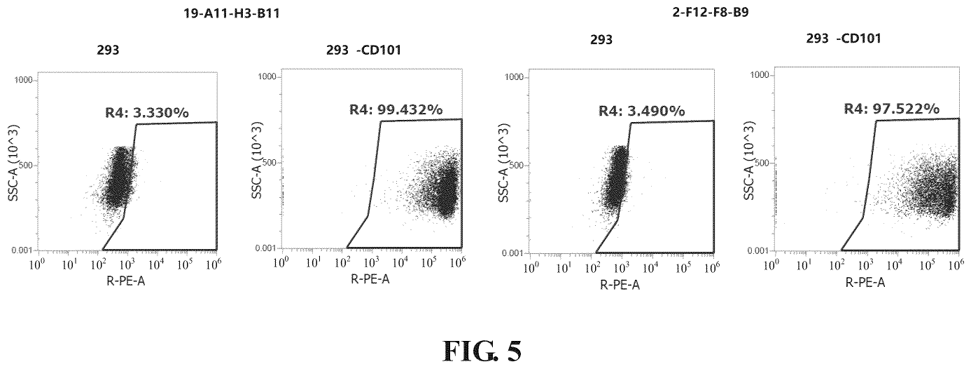

Purified chimeric antibodies were tested by FACS for an immunization titer (FIG. 5). Results showed that the two chimeric antibodies (CA1 and CA2) both could specifically bind to CD101. In the following examples, a humanized antibody design was conducted with a CA1 antibody (corresponding to 19-A11-H3-B11) as an example.

### Example 3 Construction and Detection of Humanized Antibodies

### 1. Design of Humanized Antibodies

According to the acquired amino acid sequence information of VH and VL of a murine antibody, a humanization design was conducted. With CDR sequences of the original antibody retained, different humanized antibody templates were selected for a heavy chain and a light chain according to results of germline alignment and results of antibody structure simulation. Candidate humanized antibody sequences were designed through reverse mutations for FRs after humanization (Table 2).

TABLE 2
Humanized antibody sequences

| | | SEQ ID |
|---|---|---|
| Region | Sequence | NO: |
| VH-HM1 | EVQLVQSGAEVKKPGASVKVSCKASGFTLTEYTIHWVR | 15 |
| (VH1) | QAPGQGLEWIGWFYPGSGGIKYNEKFQDRVTLTADESIST | |
| | VYMELSRLRSDDTAVYFCARHAFYRNYEYFDVWGTGT | |
| | MVTVSS | |
| VH-HM2 | EVQLVQSGAEVKKPGASVKVSCKASGFTLTEYTIHWVR | 16 |
| (VH2) | QAPGQGLEWIGWFYPGSGGIKYNEKFQDRVTLTADESAS | |
| | TVYMELSSLRSEDTAVYFCARHAFYRNYEYFDVWGTGT | |
| | MVTVSS | |
| VH-HM3 | EVQLVQSGAEVKKPGASVKVSCKASGFTLTEYTIHWVR | 17 |
| (VH3) | QAPGQGLEWIGWFYPGSGGIKYNEKFQDRVTLTADTSIST | |
| | VYMELSRLRSDDTVVYFCARHAFYRNYEYFDVWGTGT | |
| | MVTVSS | |
| VH-HM4 | EVQLVQSGAEVKKPGASVKVSCKASGFTLTEYTIHWVK | 18 |
| (VH4) | QAPGQGLEWIGWFYPGSGGIKYNEKFQDRVTLTADESSS | |
| | TVYMELSRLRSDDTAVYFCARHAFYRNYEYFDVWGTGT | |
| | MVTVSS | |
| VL-HM1 | DIVMTQAPFSLPVTPGTPASISCRSSKSLLHSNGITYLYWY | 19 |
| (VL1) | LQKPGQSPQLLIYQMSNLASGVPDRFSSSGSGTDFTLKIS | |
| | RVEAEDVGVYYCAQNLELPLTFGAGTKLEIK | |
| VL-HM2 | DIVMTQAPLSLPVTPGTPASISCRSSKSLLHSNGITYLYWY | 20 |
| (VL2) | LQKPGQSPQLLIYQMSNLASGVPDRFSSSGSGTDFTLKIS | |
| | RVEAEDVGVYYCAQNLELPLTFGAGTKLEIK | |
| VL-HM3 | DIVMTQAPFSSPVTLGTPASISCRSSKSLLHSNGITYLYWY | 21 |
| (VL3) | QQRPGQSPRLLIYQMSNLASGVPDRFSSSGSGTDFTLKIS | |
| | RVEAEDVGVYYCAQNLELPLTFGAGTKLEIK | |

### 2. Gene Synthesis and Expression Vector Construction for Humanized Antibodies

Gene synthesis was conducted for heavy and light chains of each humanized antibody designed above. A heavy chain sequence was subcloned into a pcDNA3.4-IgG1 expression vector, and a light chain sequence was subcloned into a pcDNA3.4-IgKc expression vector (schematic diagrams of the vectors were shown in FIG. 3). After vectors were verified by sequencing to be correct, endotoxin-removed plasmids were prepared.

### 3. Expression and FACS Detection of Humanized Antibodies

Expression vectors for humanized antibodies carrying a designed heavy-chain or light-chain sequence were transiently transfected into 293F cells in pairs according to a standard protocol. Specifically, PBS was added at 2 mL/well to a 6-well plate, then 2 μg of a pcDNA3.4-IgG1 expression vector and 2 μg of a pcDNA3.4-IgKc expression vector were added, and thorough mixing was conducted by pipetting up and down. 12 μL of a LVTransm transfection reagent was added, then thorough mixing was immediately conducted by pipetting up and down, and the plate was allowed to stand at room temperature for 10 min. A DNA/LVTransm complex was added to 3 mL of 293F cells, and gentle shaking was conducted for thorough mixing. The cells were further cultured for 3 d in a 37° C. and 5% CO₂ incubator, and a culture supernatant was collected through centrifugation and tested by FACS.

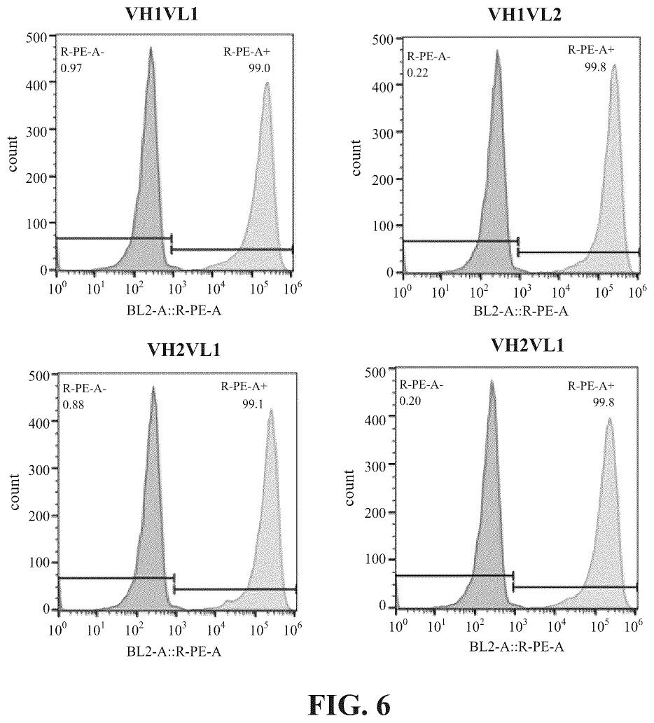

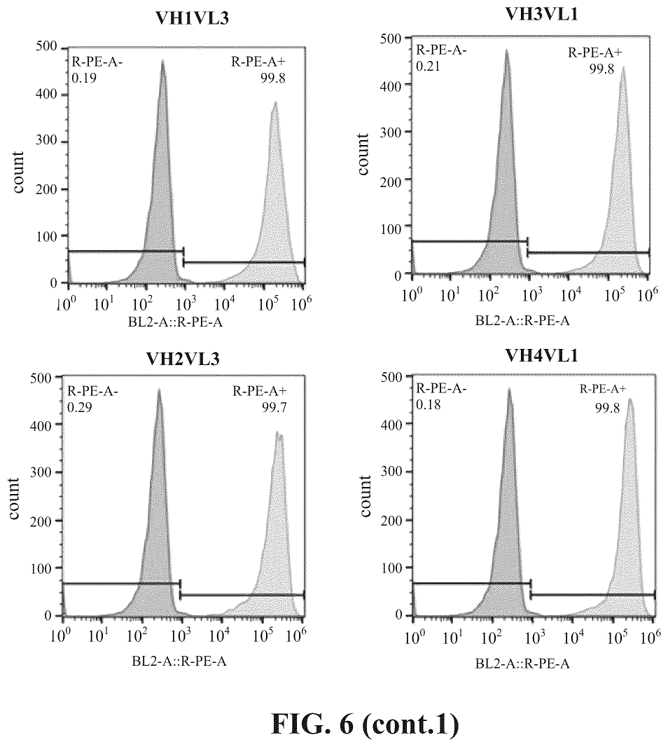

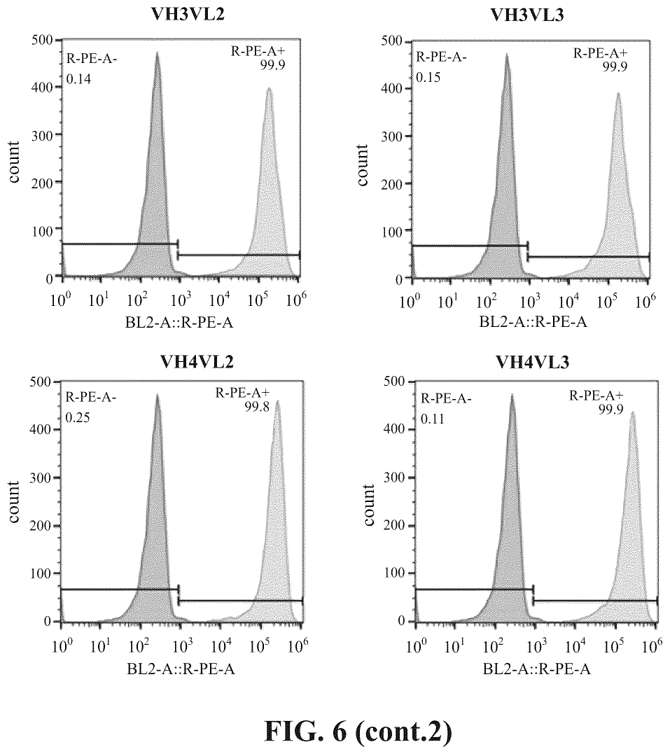

FACS was conducted according to a standard protocol. In short, 3×10⁵ 293F-CD101 cells in a logarithmic growth phase and 100 μL of a humanized antibody-containing supernatant were fully mixed and incubated for 1 h at room temperature. Cells were washed with PBS 3 times. 100 μL of a secondary antibody (PE-anti-human IgG, which was diluted 1:200) was added, thorough mixing was conducted, and incubation was conducted for 45 min at room temperature in the dark. Cells were washed with PBS 3 times and then resuspended with 500 μL of PBS. A fluorescence intensity was read by a multimode microplate reader for FACS analysis. Detection results were shown in FIG. 6. Results showed that the VH1-VL1, VH1-VL2, VH1-VL3, VH2-VL1, VH2-VL2, VH2-VL3, VH3-VL1, VH3-VL2, VH3-VL3, VH4-VL1, VH4-VL2, and VH4-VL3 combinations all could bind to CD101 with high affinity, and the binding was basically the same as the binding of the chimeric antibodies. In the follow-up experiments, the four combinations of VH1-VL1, VH1-VL2, VH3-VL1, and VH3-VL2 (hu.CA1 to hu.CA4) were taken as examples for antibody purification and affinity testing.

### 4. Expression, Purification, and Affinity Testing for Humanized Antibodies

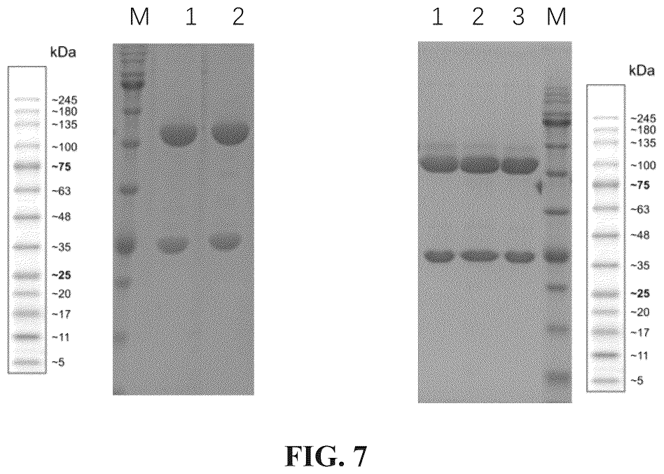

With reference to the protocol for antibody expression and purification in Example 2, the pcDNA3.4-IgG1 and pcDNA3.4-IgKc expression vectors corresponding to the selected antibody heavy/light chain combinations each were transfected into 293F cells with a LVTransm transfection reagent. The cells were cultured, and a resulting culture supernatant was collected through centrifugation. An antibody was purified with a Protein A affinity column, and a concentration of a purified antibody was determined with Nanodrop. In addition, SDS-PAGE was conducted. SDS-PAGE results were shown in FIG. 7.

100 μL of each of purified hu.CA1 to hu.CA4 (10 μg/mL, 3-fold diluted to 7 points) was incubated with 293F-CD101 cells. Then, with a purified human-mouse chimeric antibody CA1 constructed with VH and VL of the original murine antibody as a control, the affinity of a purified humanized antibody was detected by FACS (with reference to the FACS protocol described in this example).

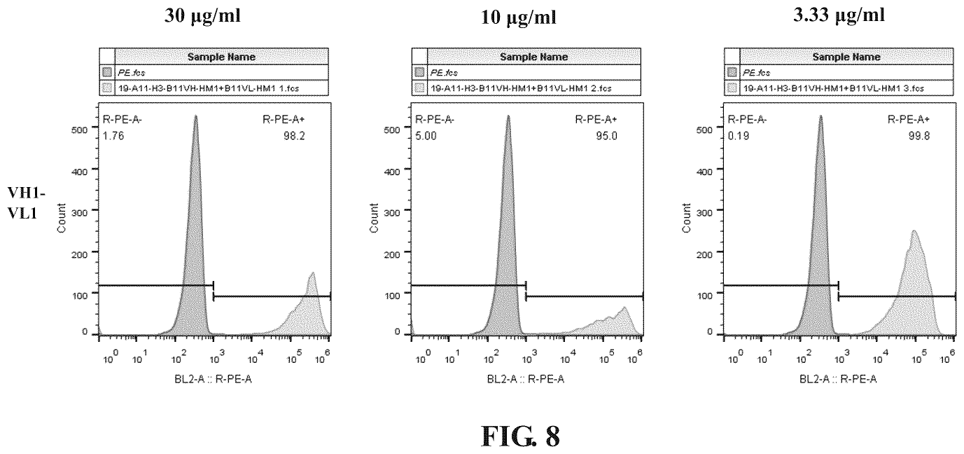

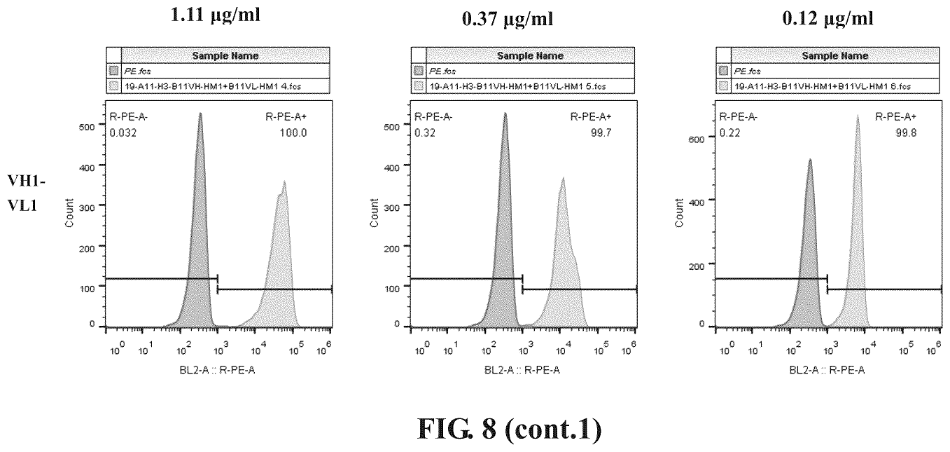

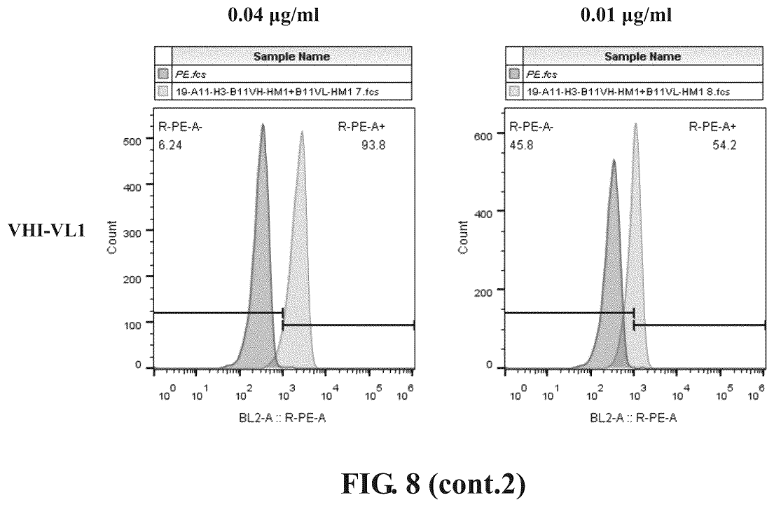

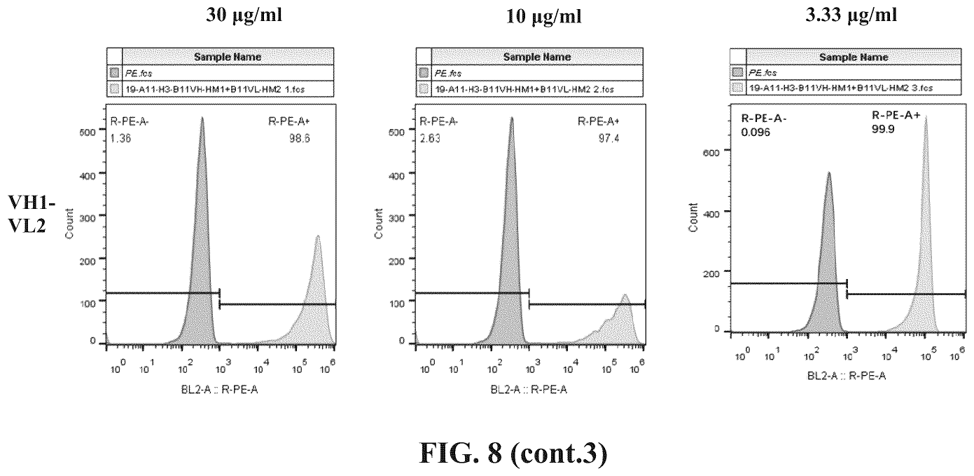

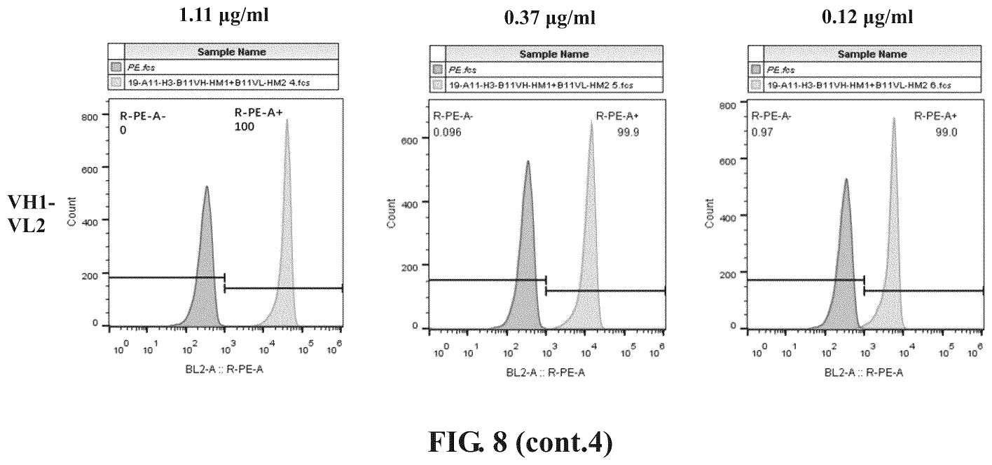

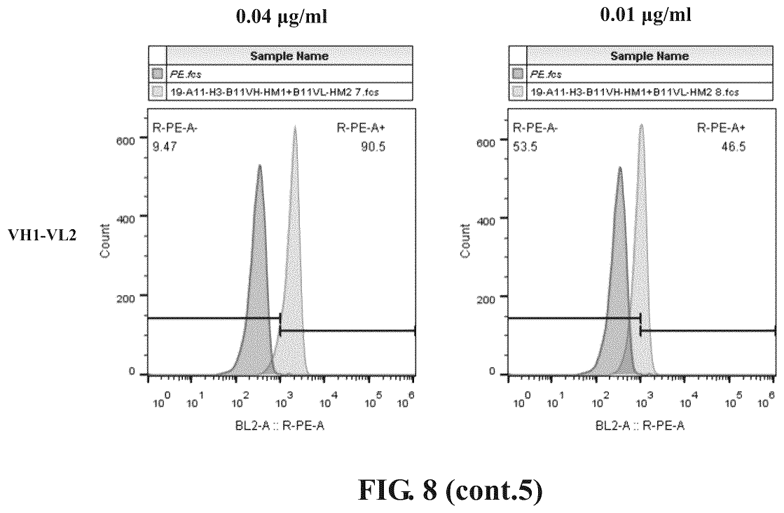

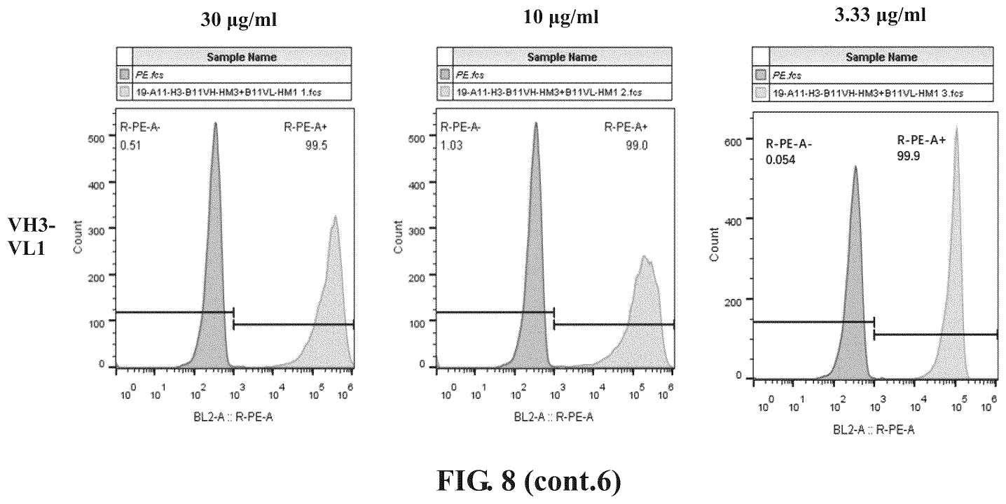

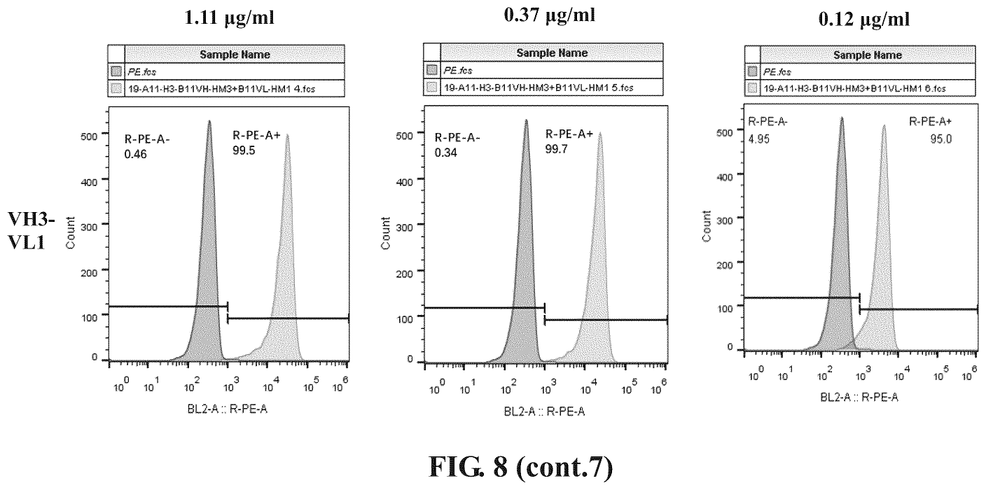

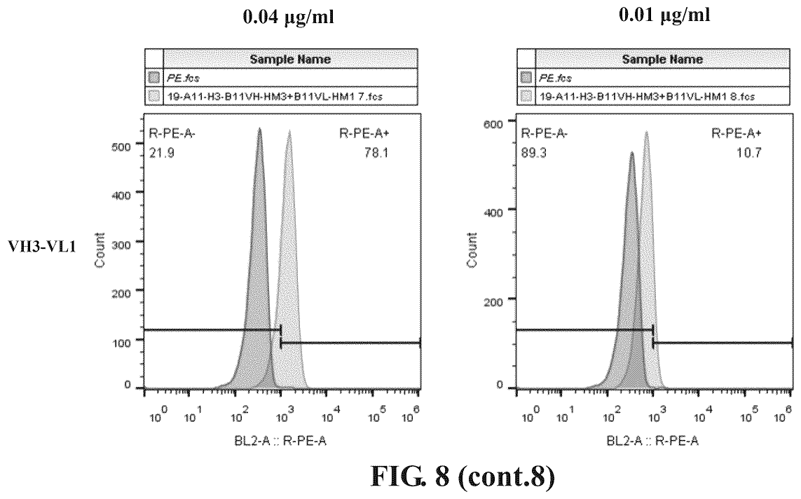

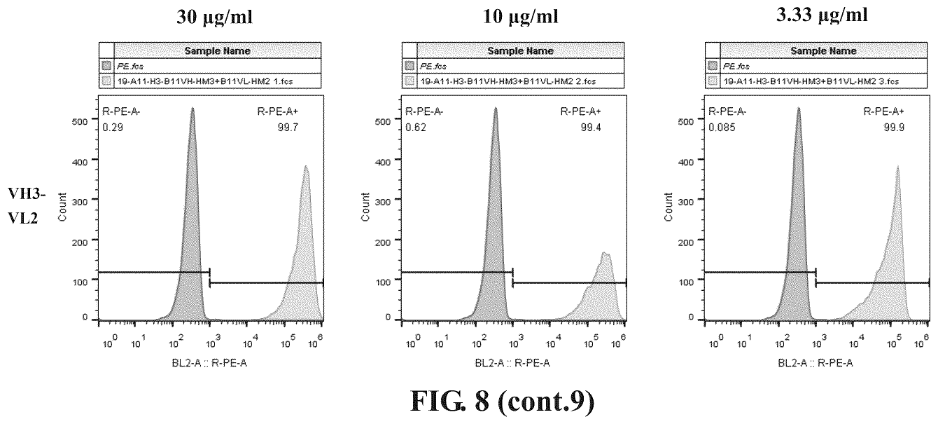

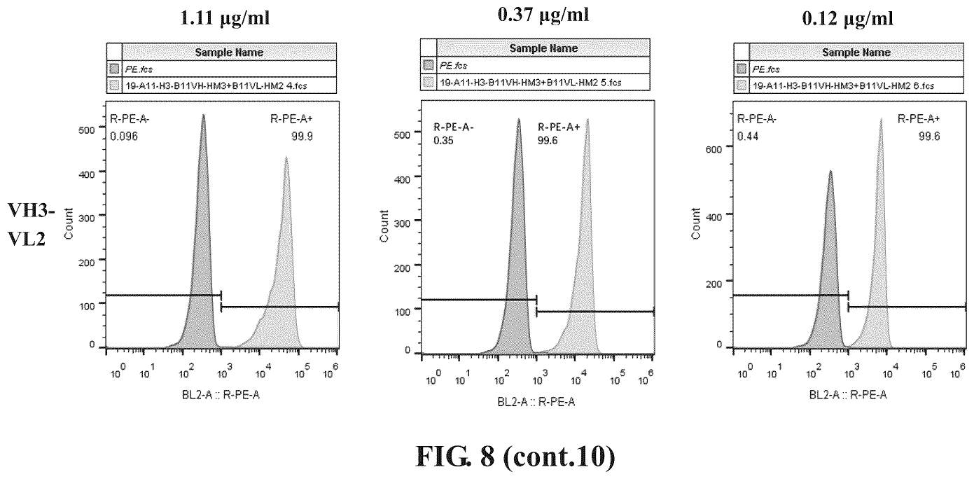

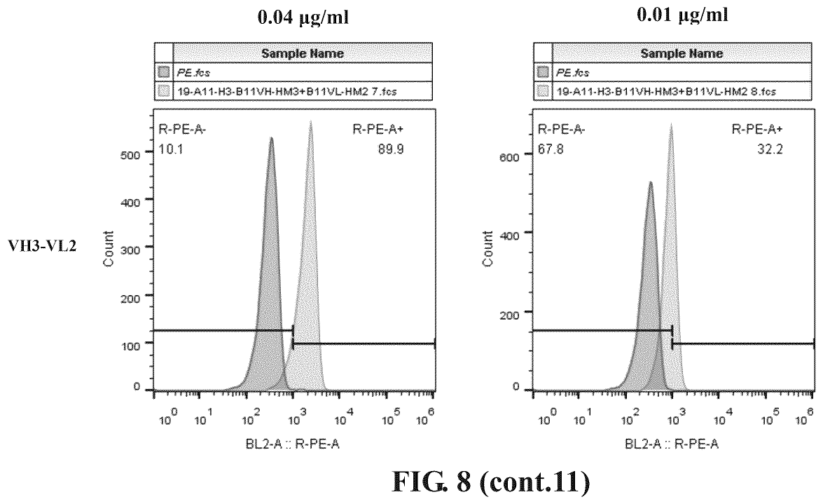

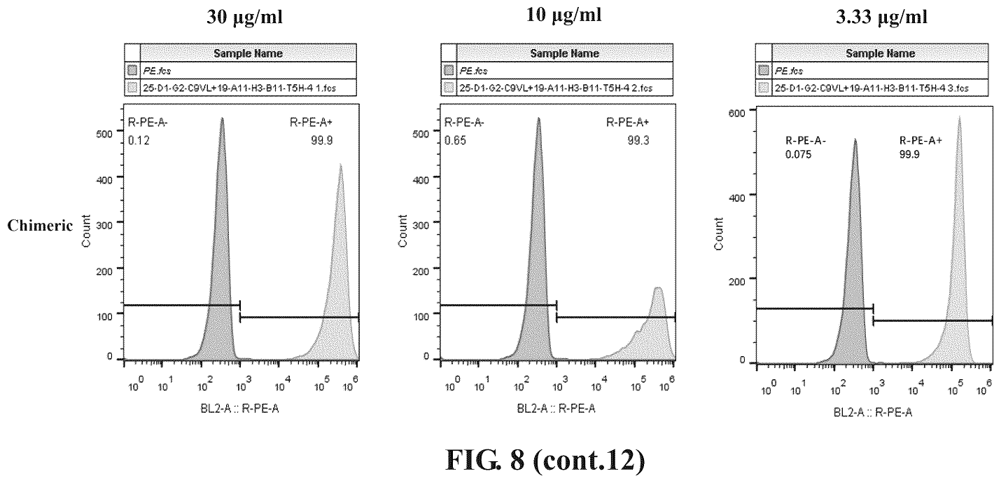

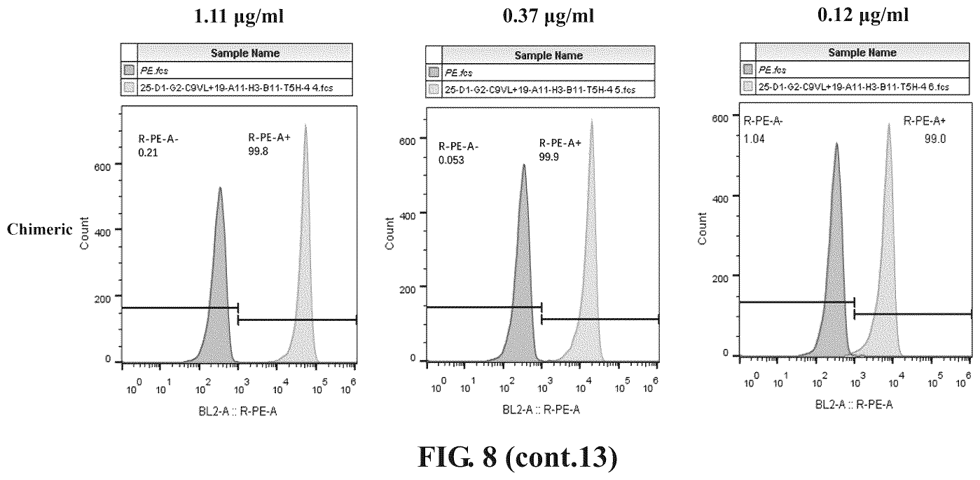

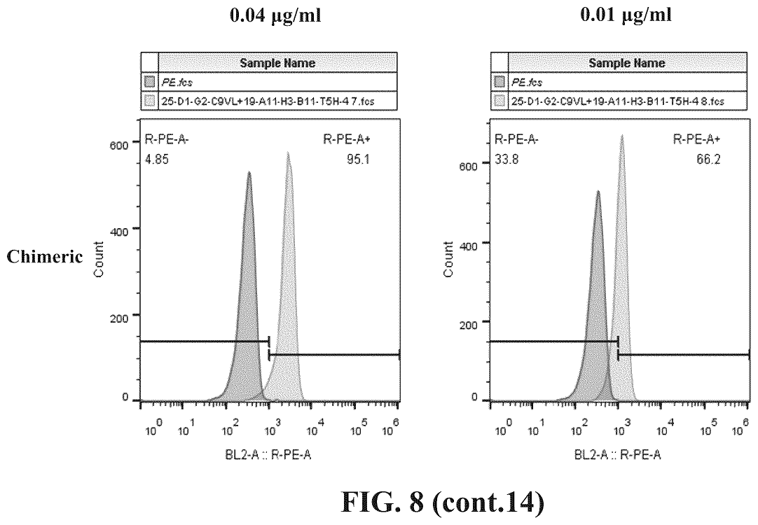

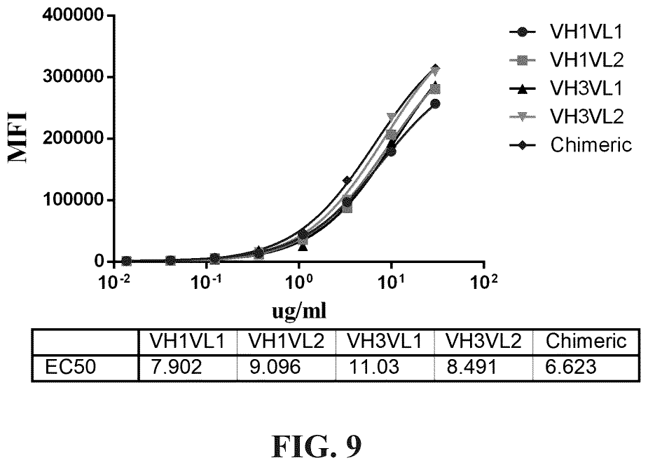

Results (FIG. 8 and FIG. 9) showed that the humanized antibodies and the chimeric antibodies exhibited consistent affinity.

### Example 4 Inhibitory Effects of a Chimeric Antibody and Humanized Antibodies for CD101

### 1. Preparation of a Stable Jurkat Cell Line Overexpressing CD101

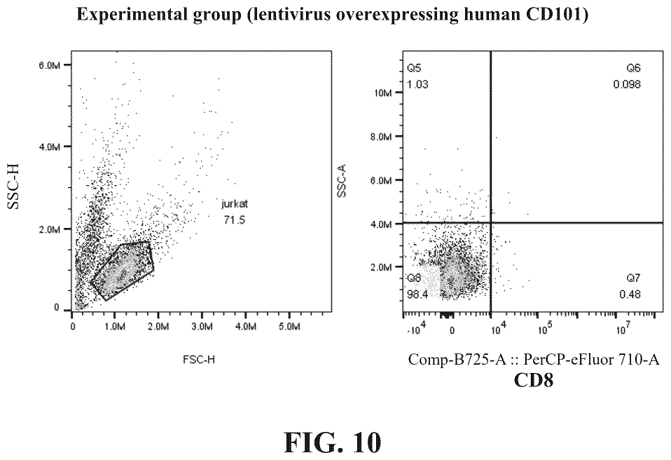

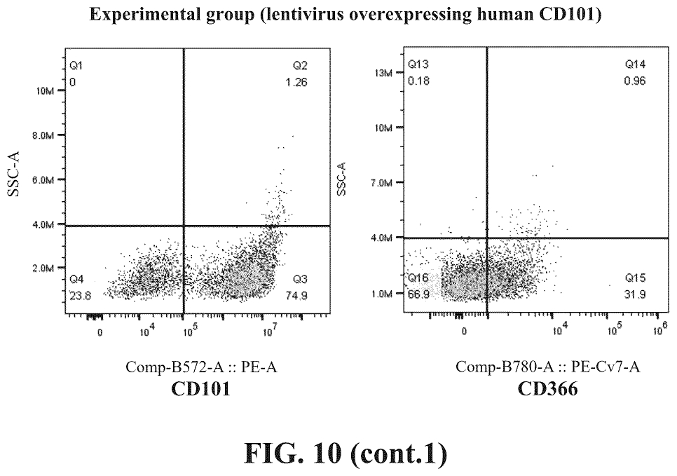

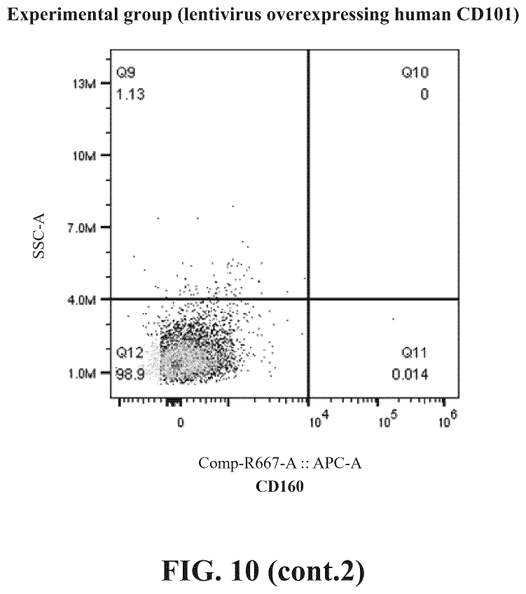

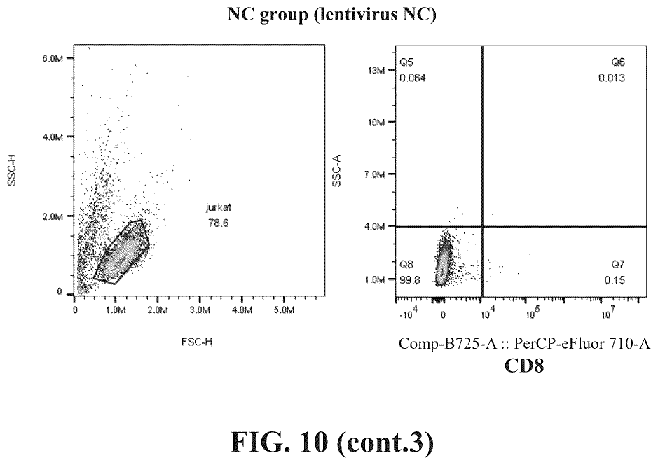

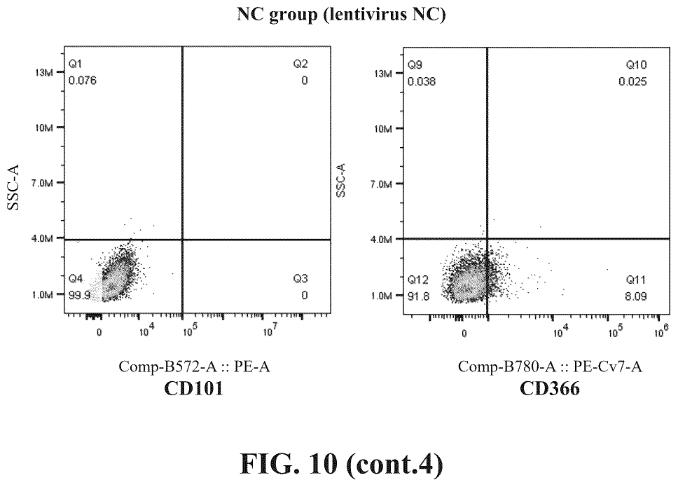

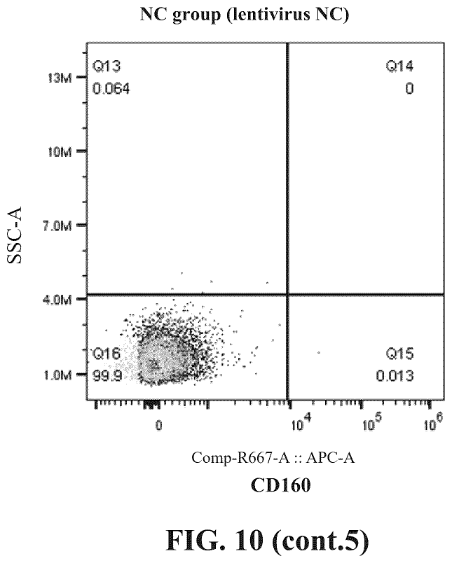

Jurkat cells (the Cell Bank of the Chinese Academy of Sciences: CSTR: 19375.09.3101HUMTCHU123) were infected with an NC lentivirus PMT290 and a human CD101-overexpressing lentivirus PSE5799 (SANGO BIOTECH, 220715), and a stable cell line was selected with puromycin. The prepared cell line was incubated with each of a CD8a monoclonal antibody (PerCP-eFluor 710 anti-mouse CD8a, eBioscience™, 46-0086-42, 100 μg), a CD101 monoclonal antibody (PE anti-human CD101 (BB27), Biolegend®, 331012, 100 μg), a CD366 monoclonal antibody (PE/Cyanine7 anti-human CD366 (Tim-3), Biolegend®, 345014, 100 μg), and a CD160 monoclonal antibody (APC anti-human CD160, Biolegend®, 341208, 100 μg), and the expression of a target gene was detected by FACS. As shown in FIG. 10, the stable Jurkat cell line overexpressing CD101 had been successfully prepared.

2. Inhibition on CD101 with Chimeric Antibodies and Humanized Antibodies

The stable Jurkat cell line carrying a CD101 target prepared above was inoculated at 1.5×10⁶ cells/2 mL/well into a 6-well plate and incubated in a 37° C. incubator for 24 h. Then a chimeric antibody (CA1) and humanized antibodies (hu.CA1 to hu.CA4) each were added at 6 concentrations (4,000 ng/mL, 1,600 ng/ml, 640 ng/ml, 256 ng/ml, 102.4 ng/mL, and 40.96 ng/mL), and PBS was added in an NC group. The plate was further incubated in a 37° C. incubator. At 3 h and 8 h after antibody addition, a sample was collected for flow cytometry with detection indexes of CD8, CD366, and CD101. The antibody information involved was shown in Table 3 below.

TABLE 3
Antibody information detected by flow cytometry

| Product name | Clone | Isotype | Brand | Item No. | Specification |
|---|---|---|---|---|---|
| CD101 Monoclonal | Moushi101 | Rat/IgG2a, | eBioscience ™ | 12-1011-82 | 100 μg |
| Antibody (Moushi101), PE, | | kappa | | | |
| eBioscience | | | | | |
| CD8a Monoclonal Antibody | 53-6.7 | Rat/IgG2a, | eBioscience ™ | 46-0081-82 | 100 μg |
| (53-6.7), PerCP-eFluor 710, | | kappa | | | |
| eBioscience | | | | | |
| PE/Cyanine7 anti-mouse | B8.2C12 | Rat IgG1, K | BLG | 134010 | 100 μg |
| CD366 (Tim-3) Antibody | | | | | |

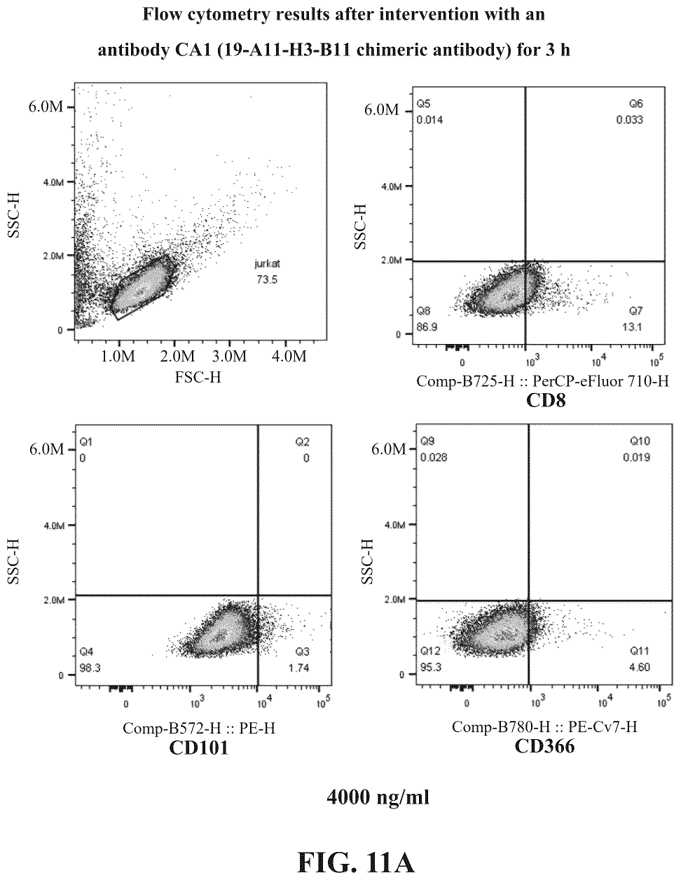

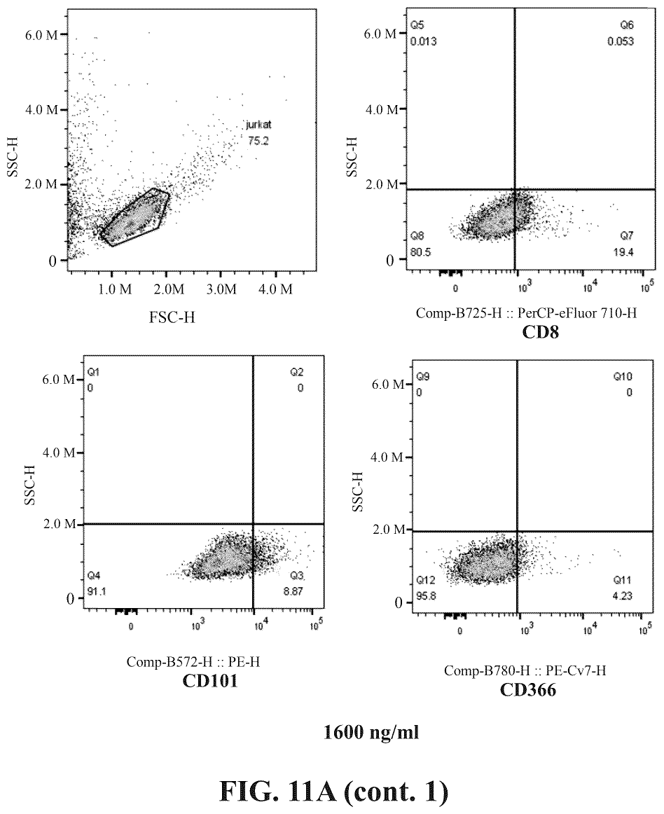

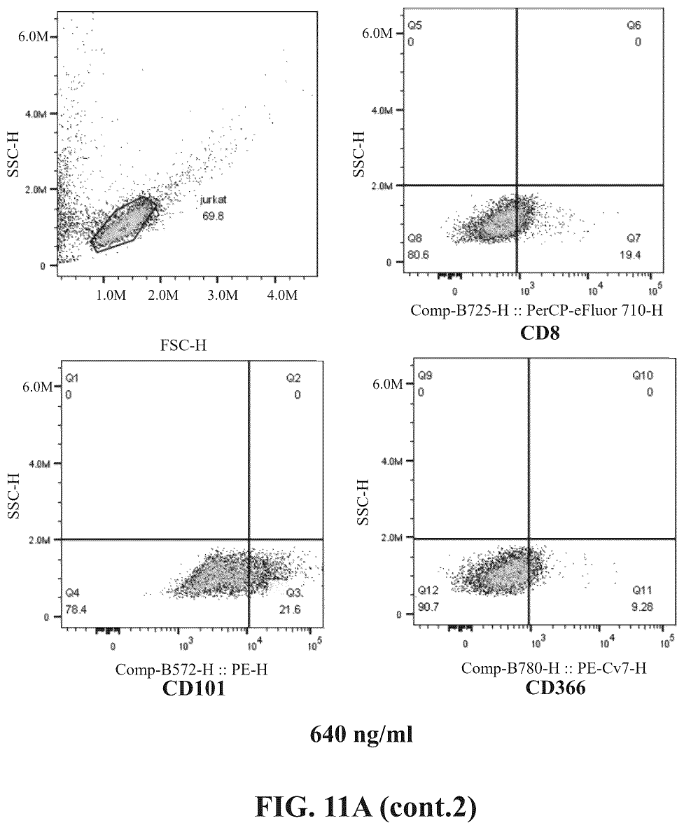

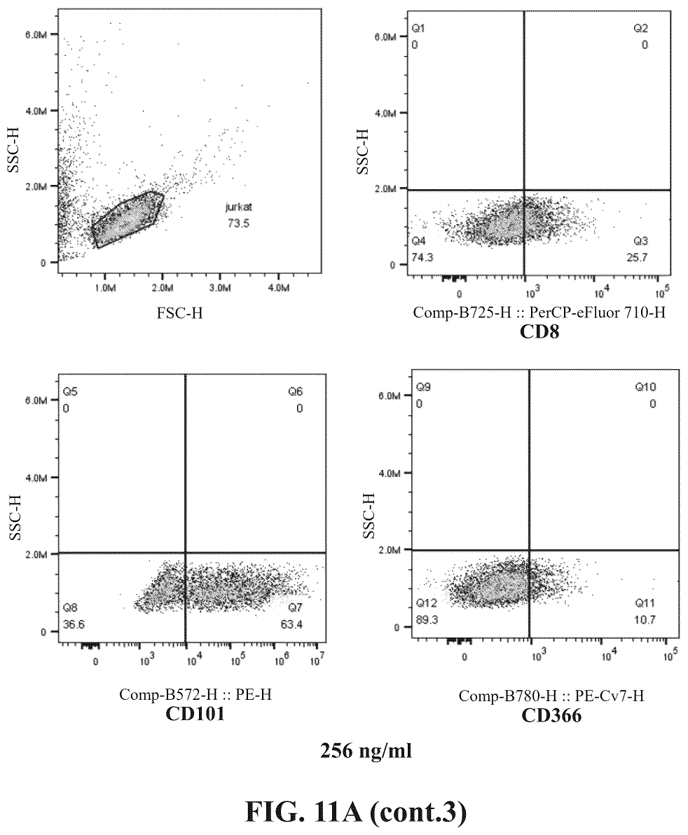

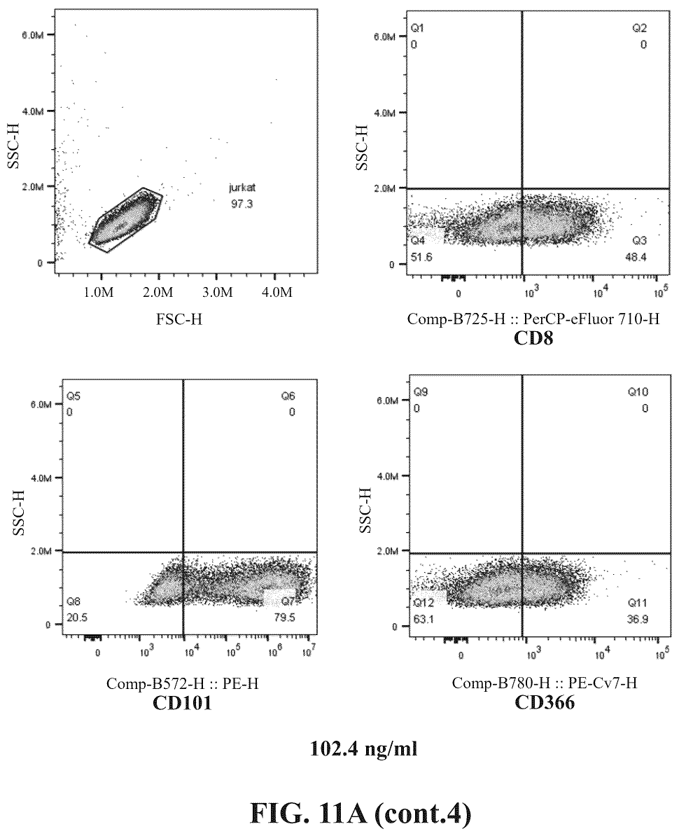

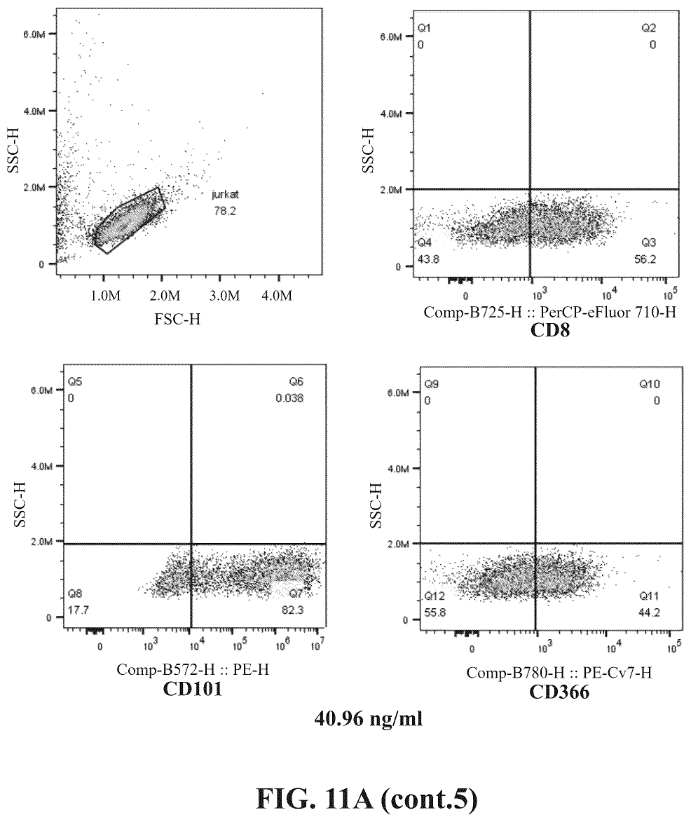

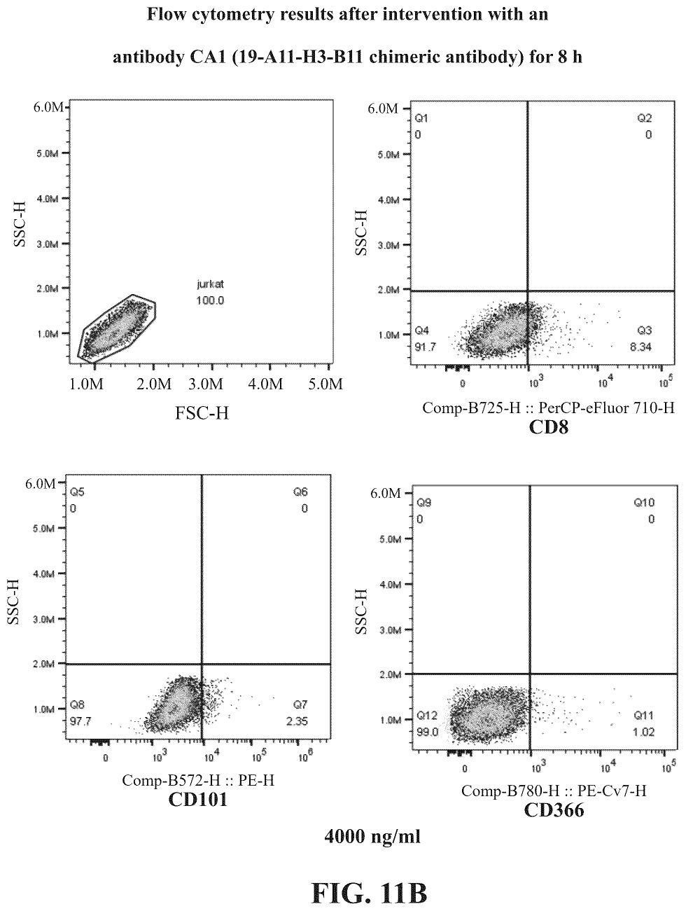

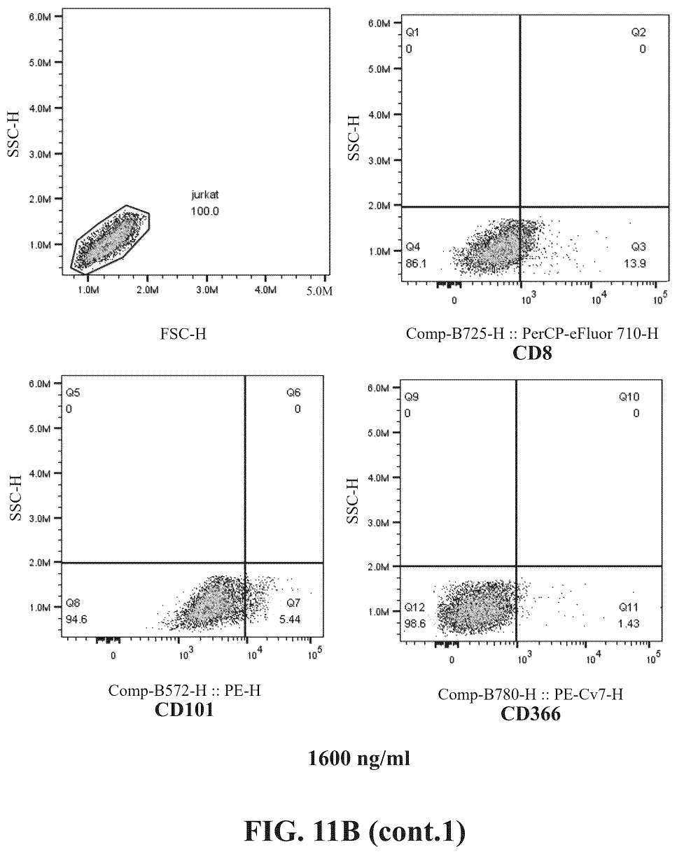

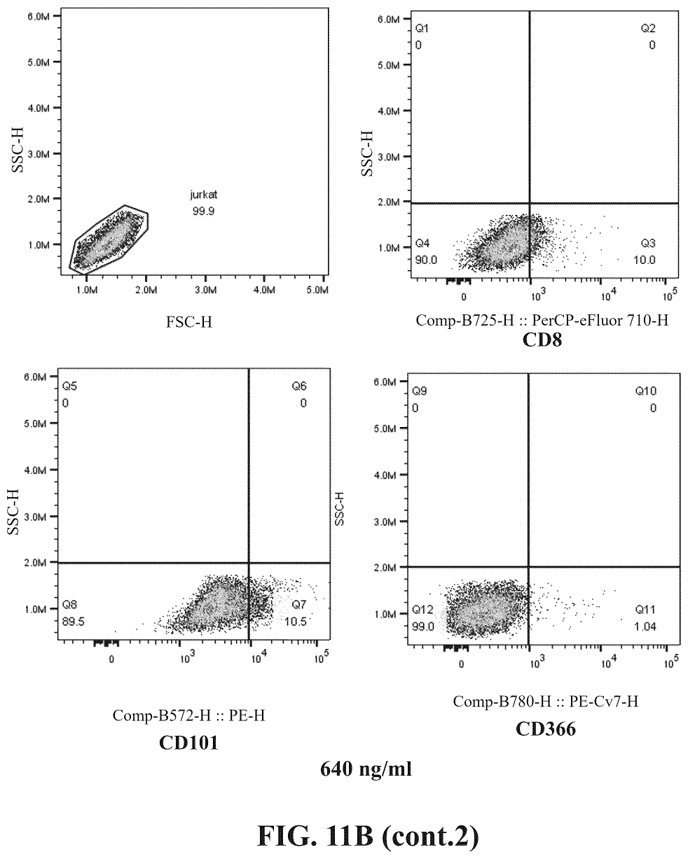

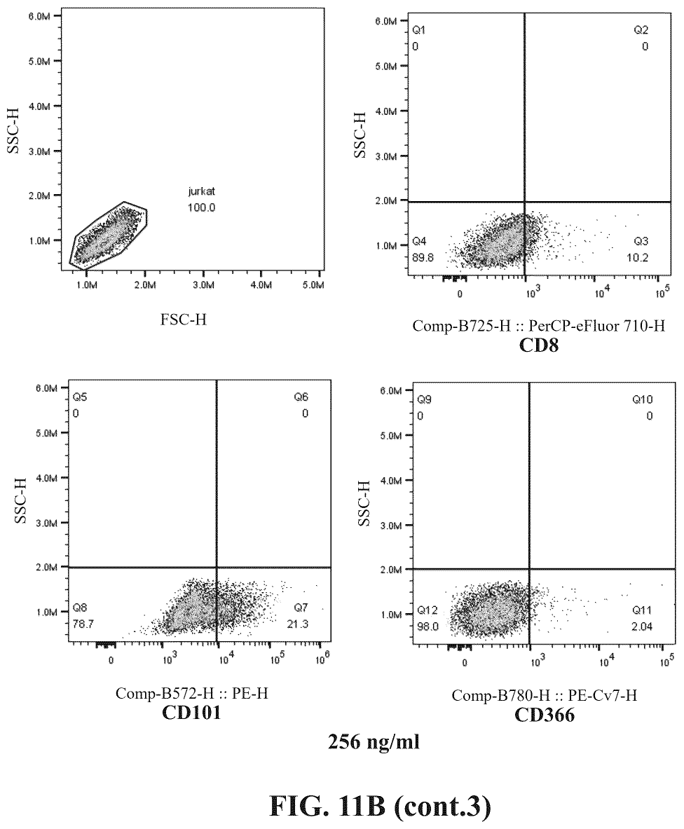

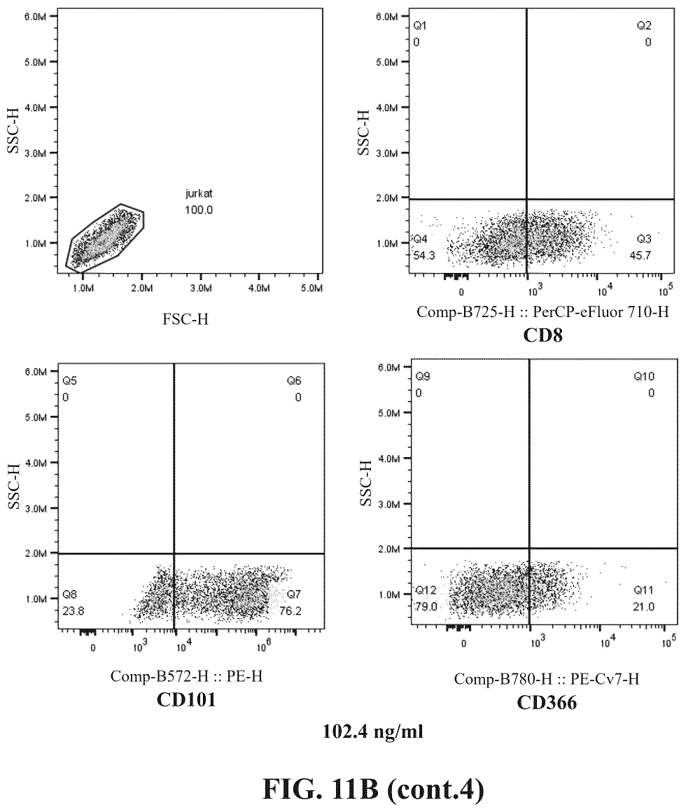

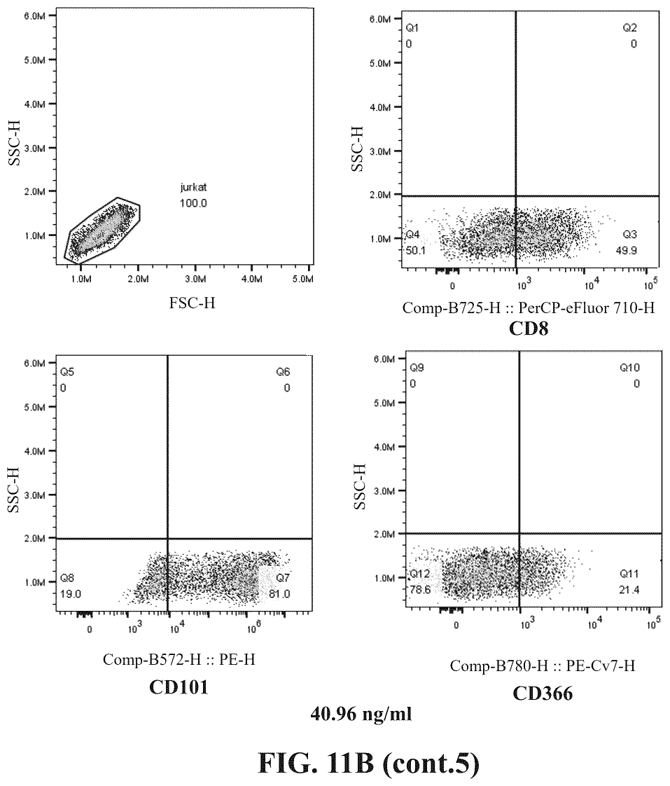

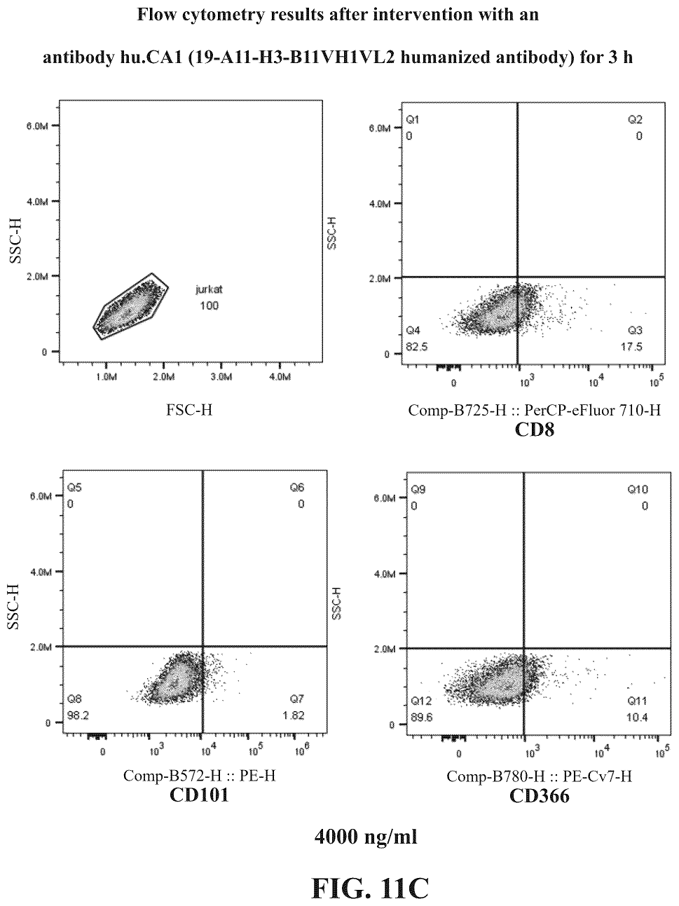

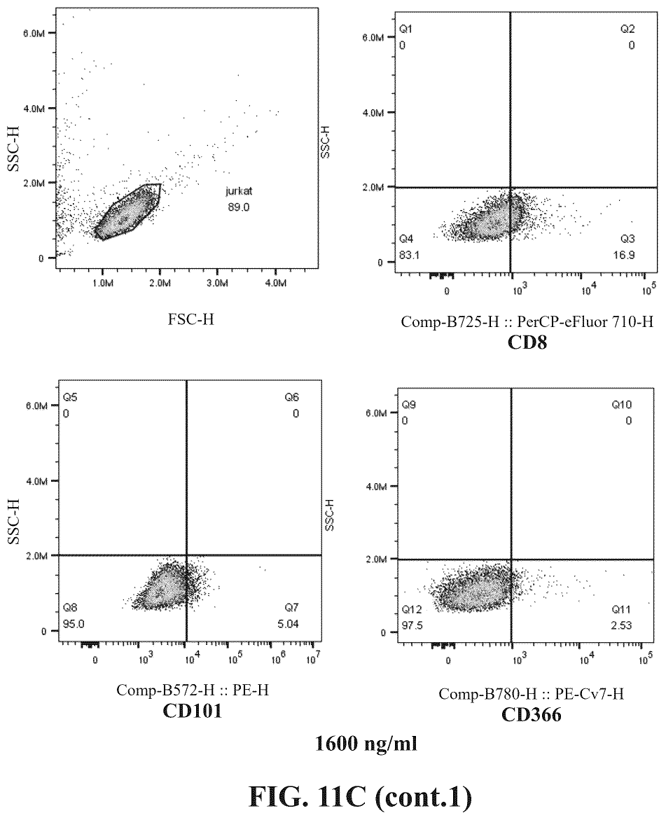

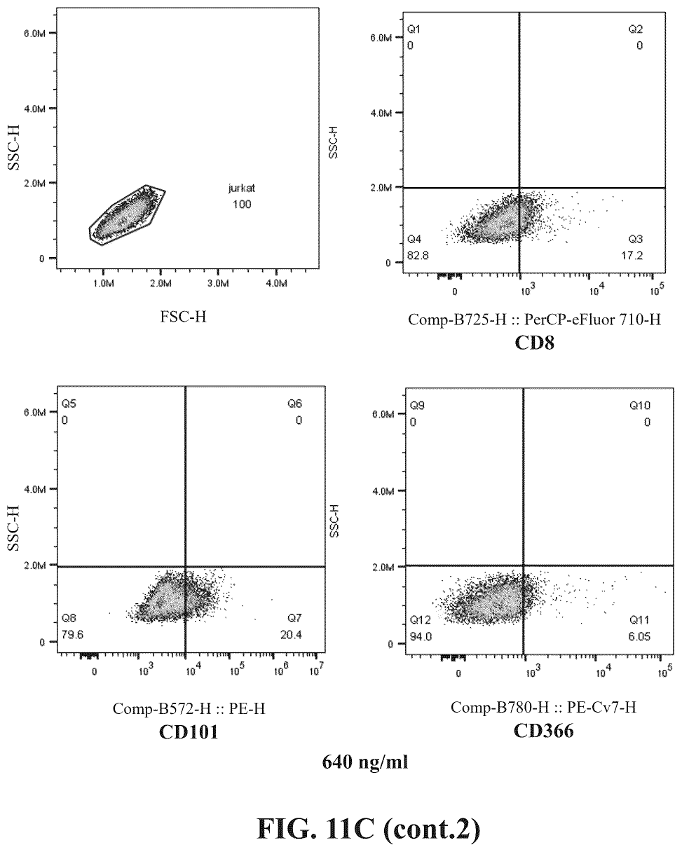

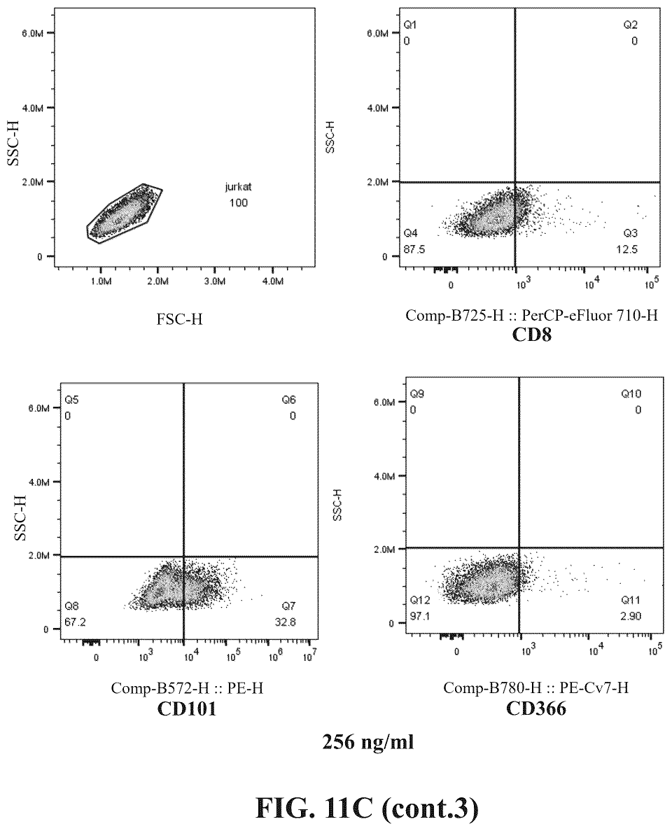

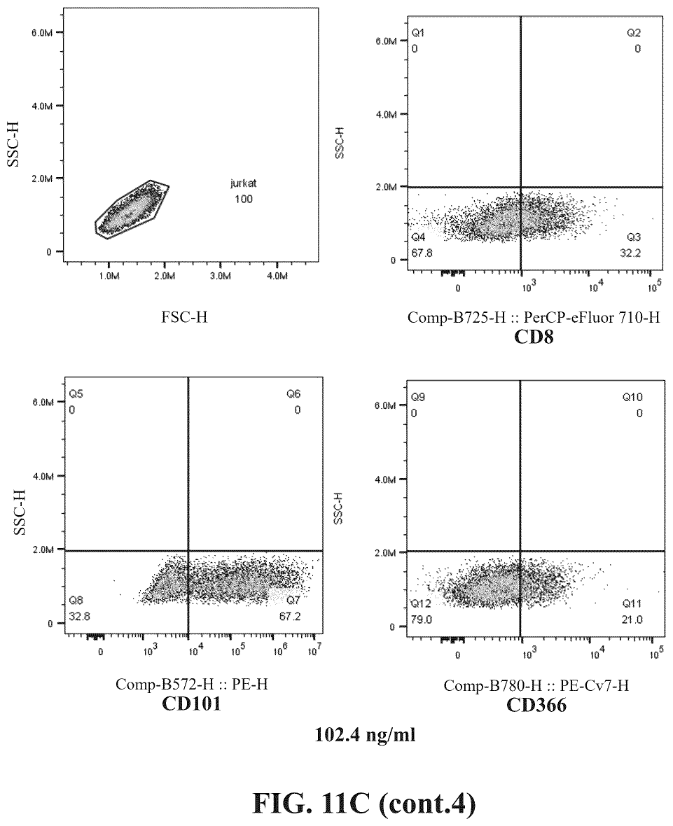

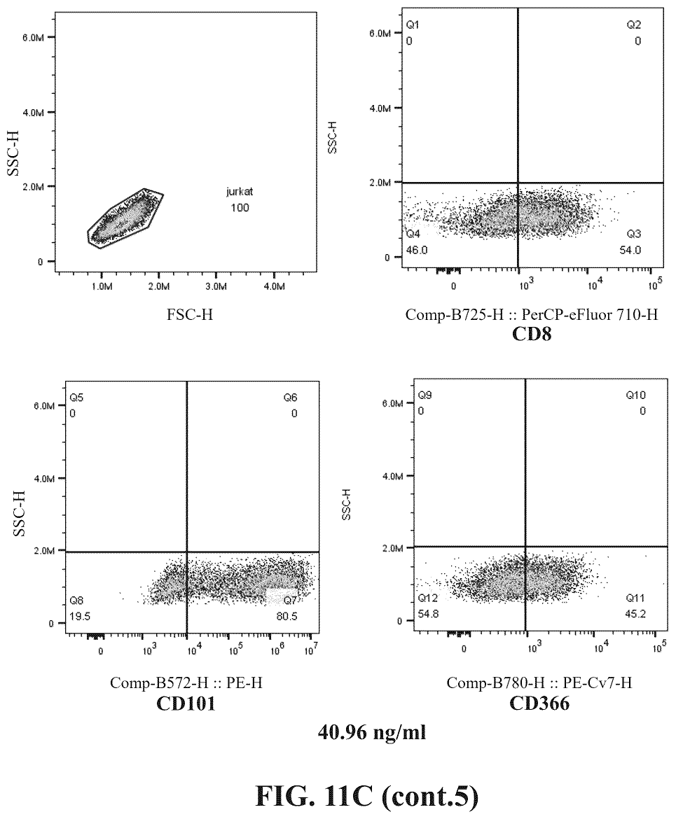

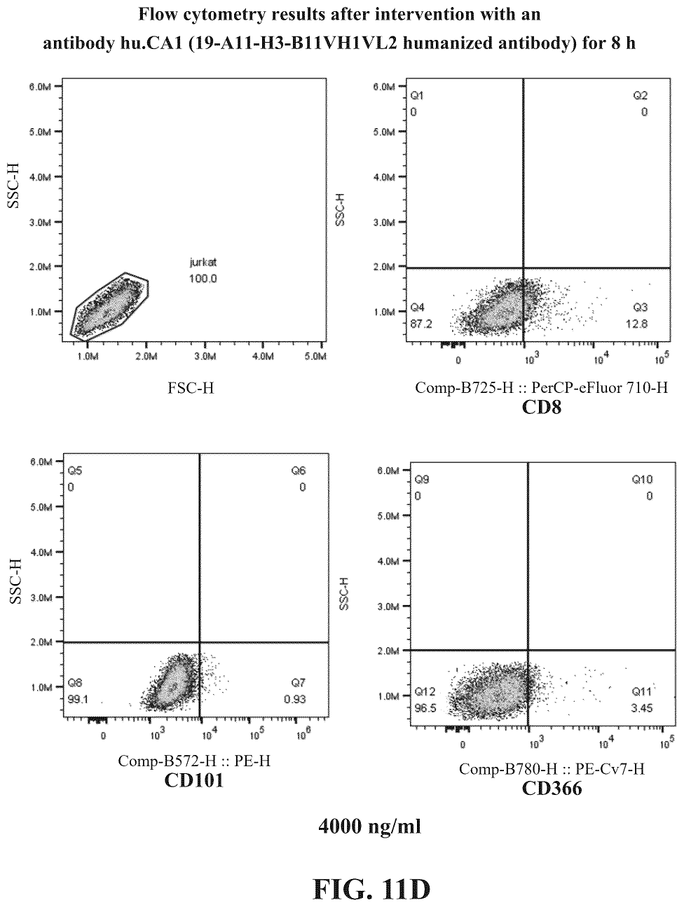

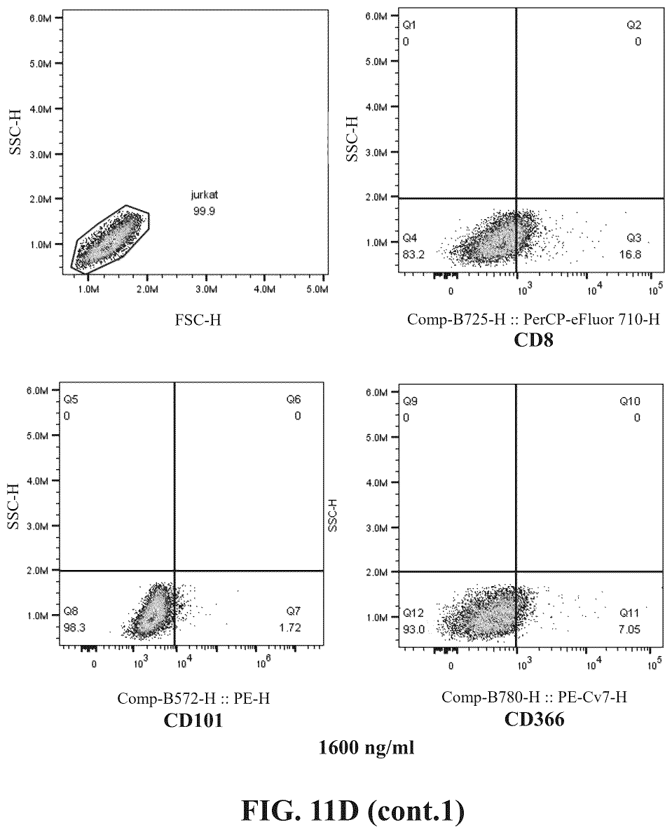

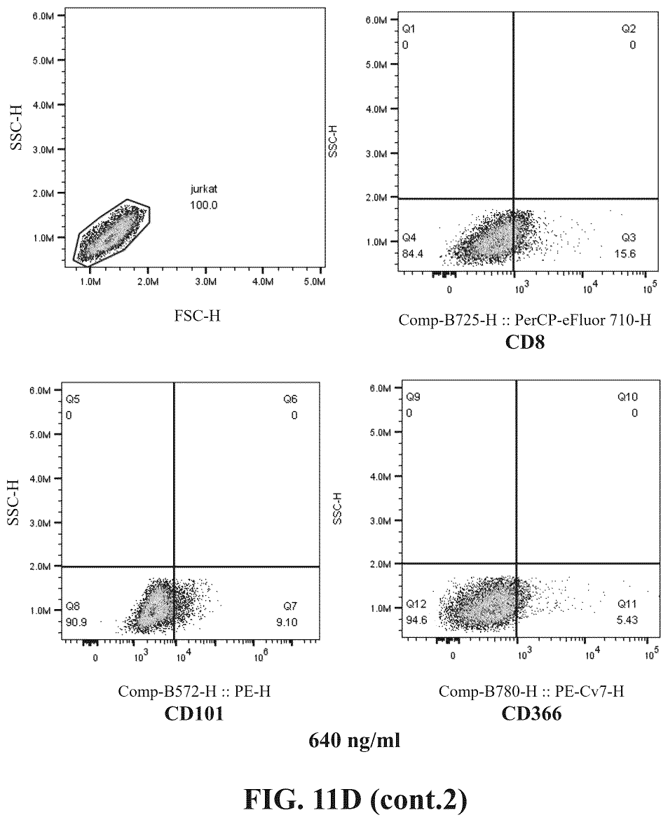

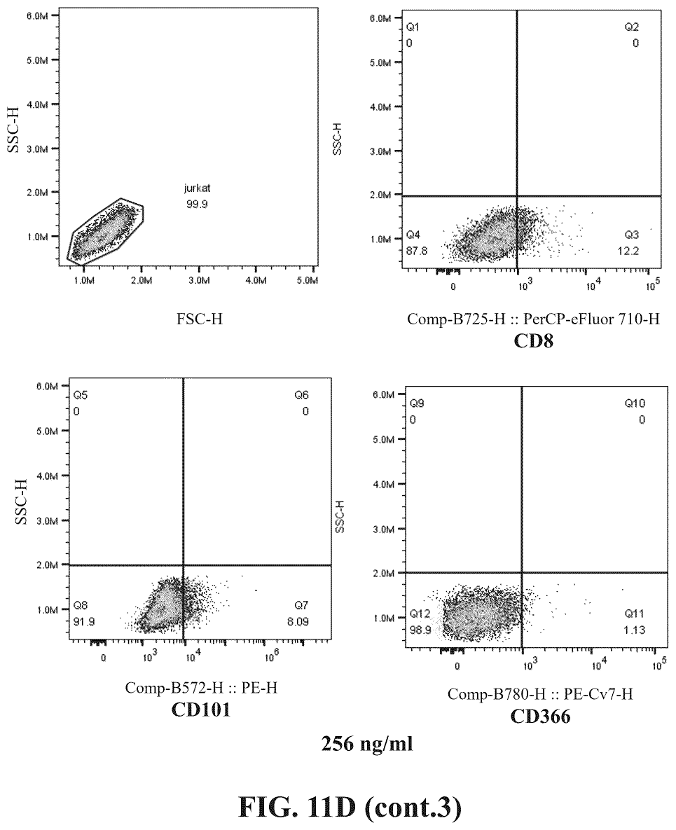

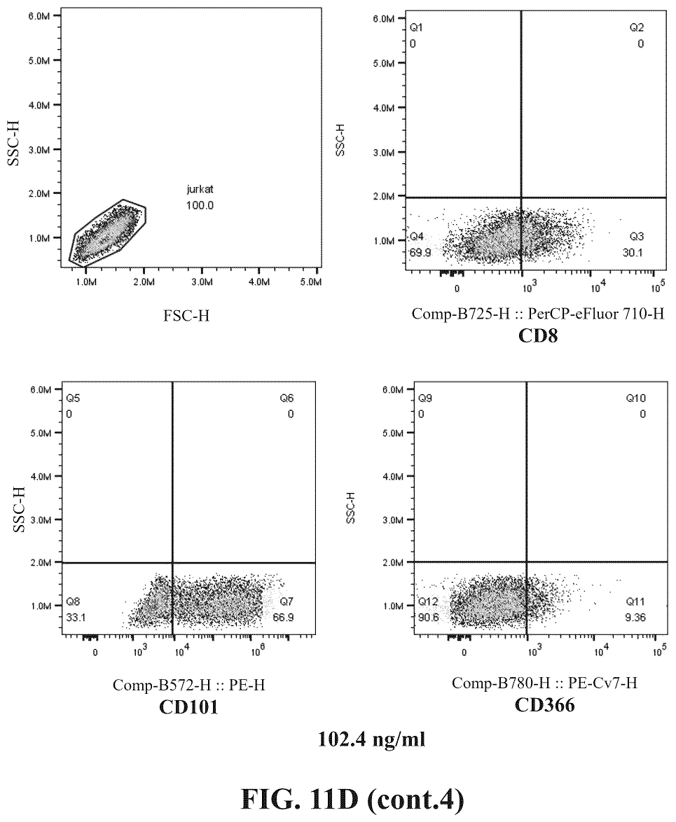

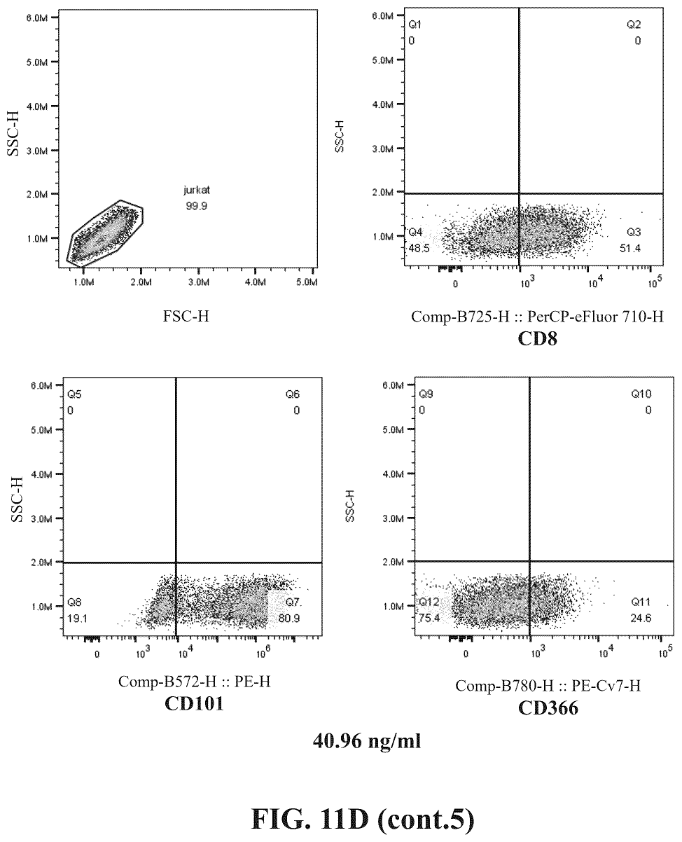

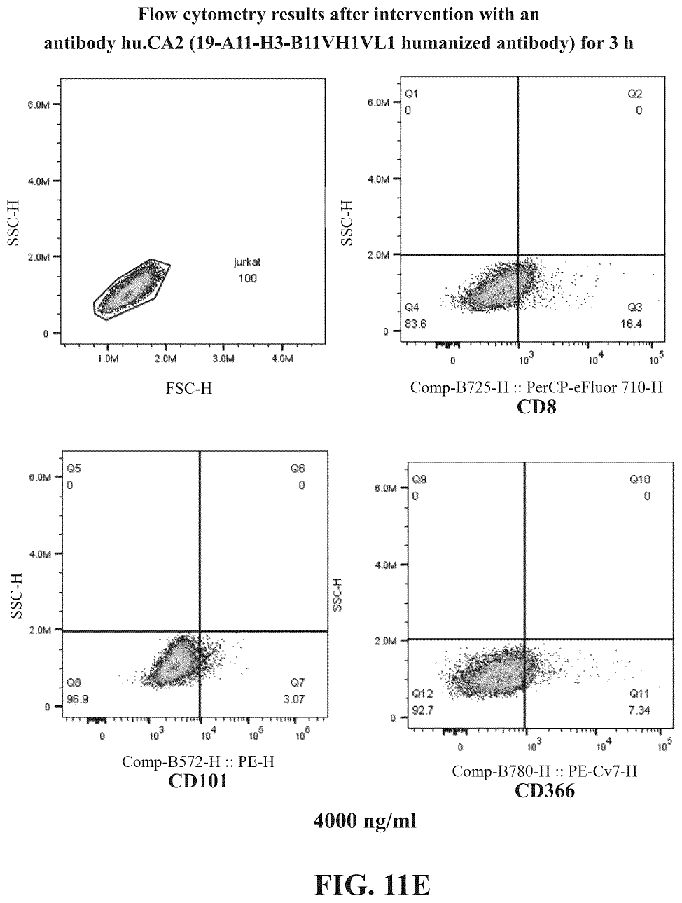

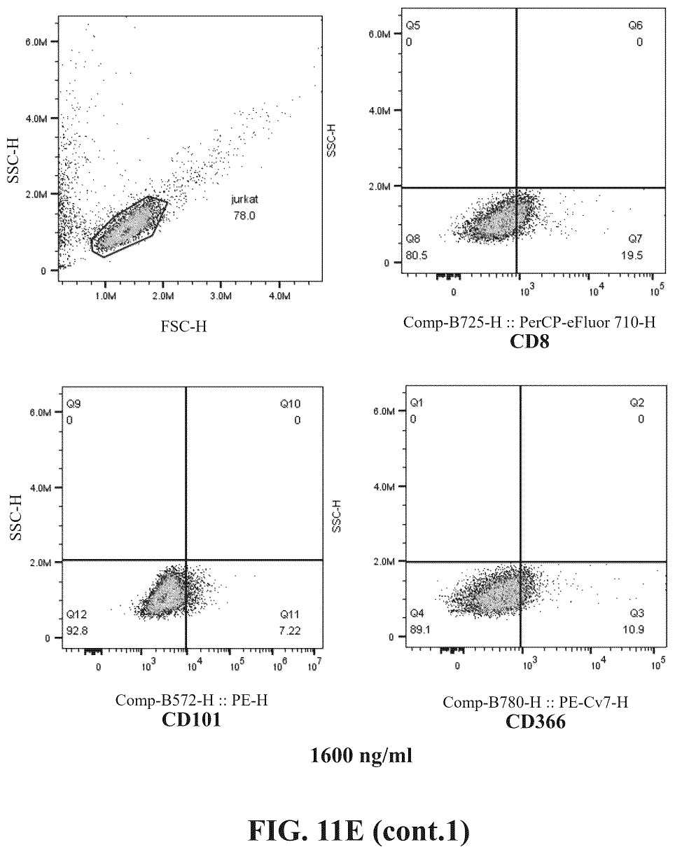

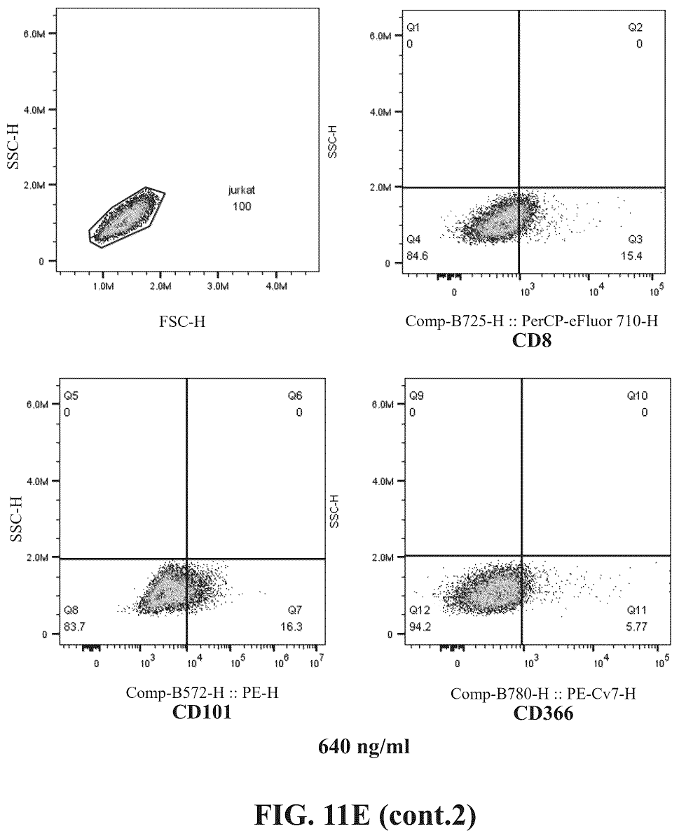

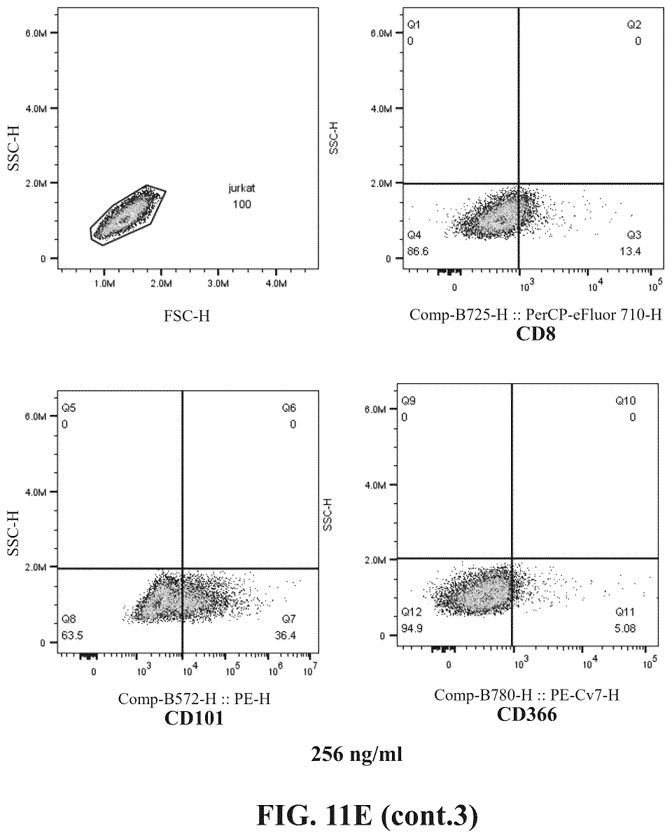

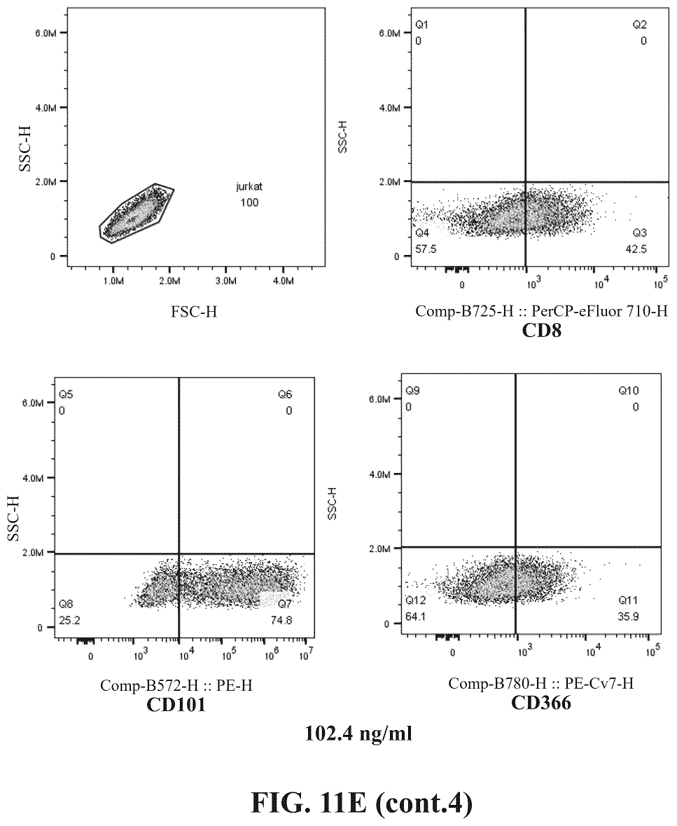

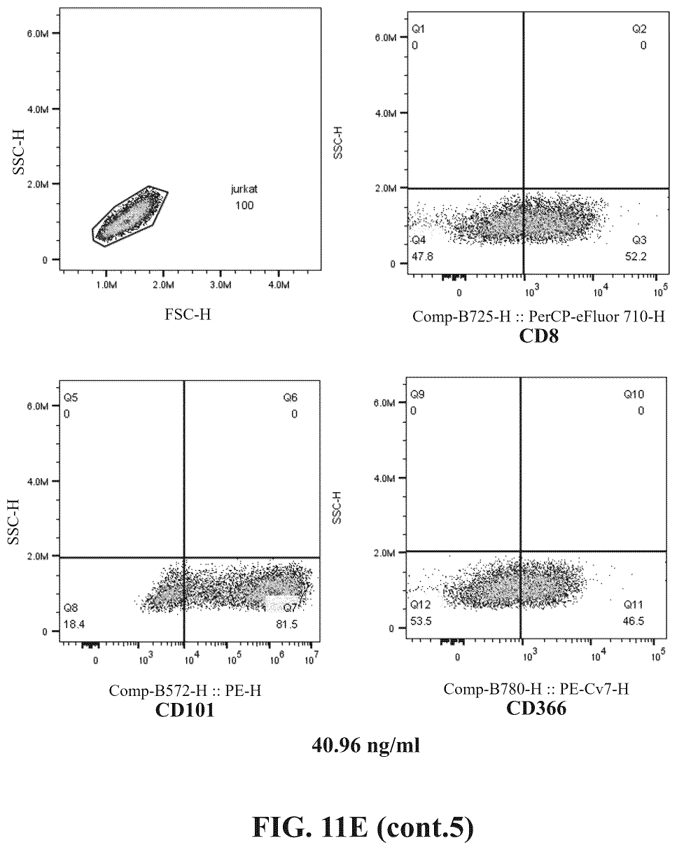

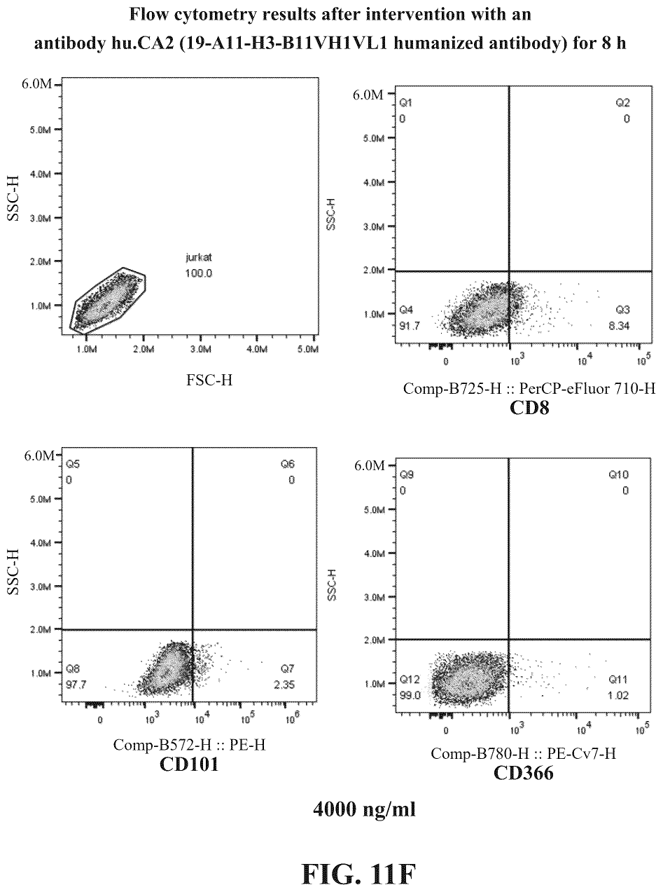

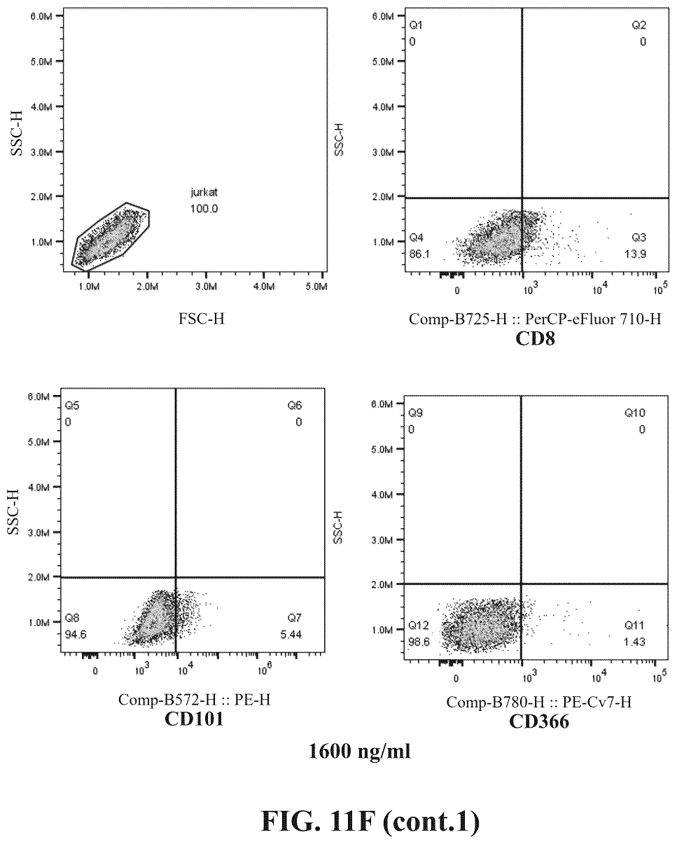

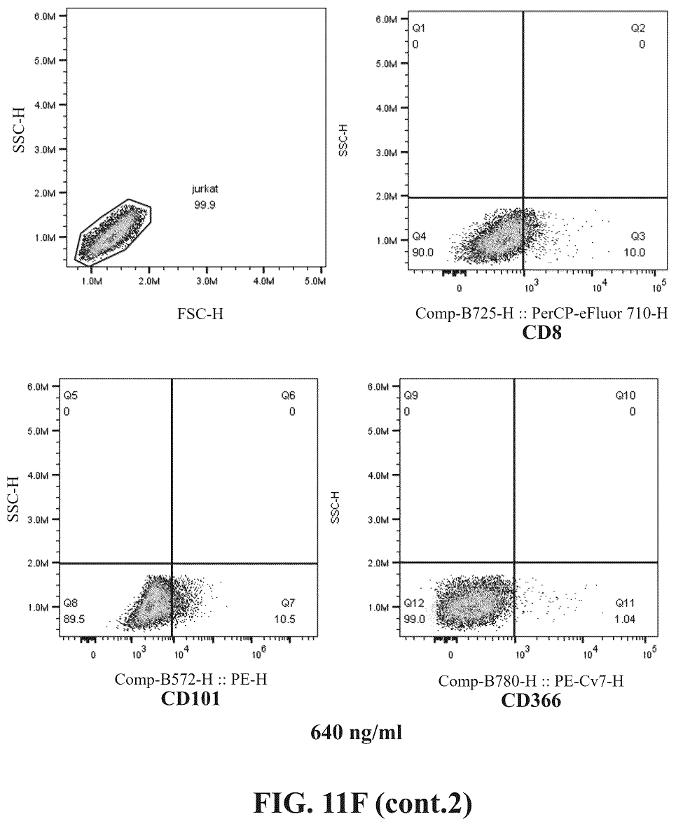

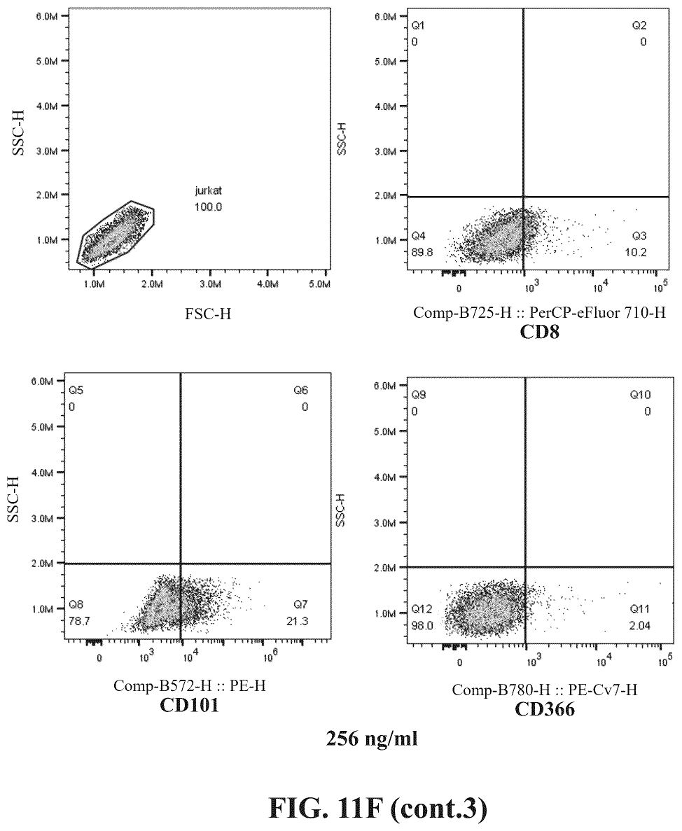

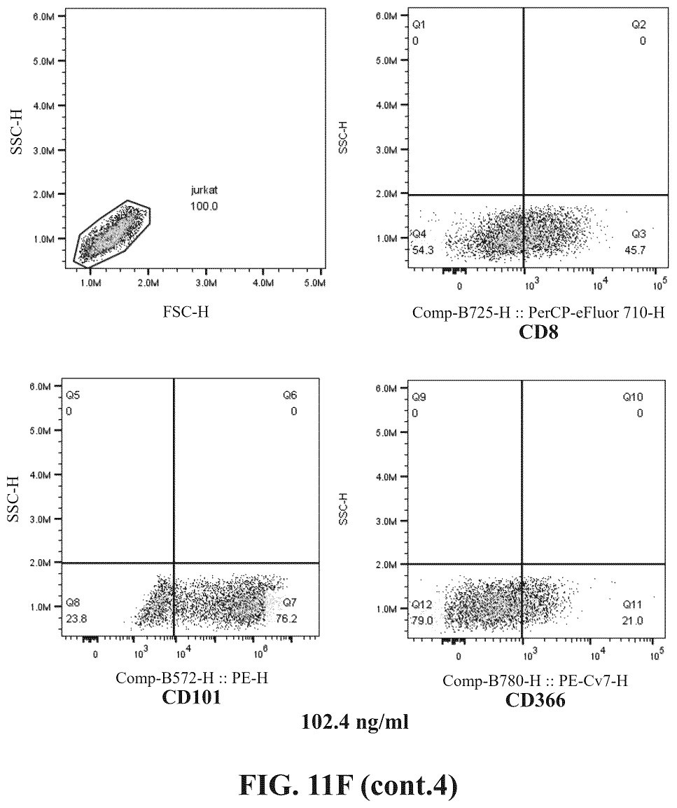

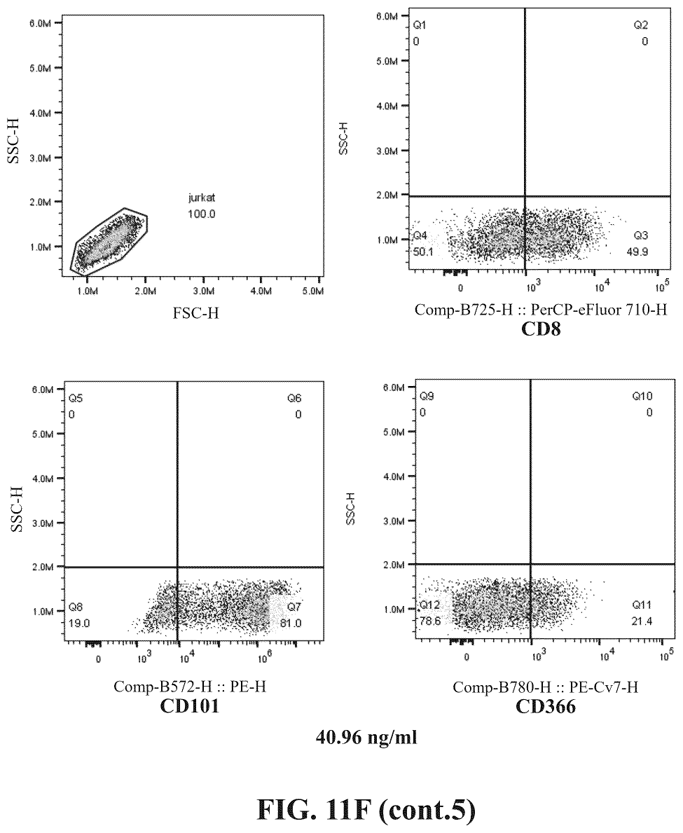

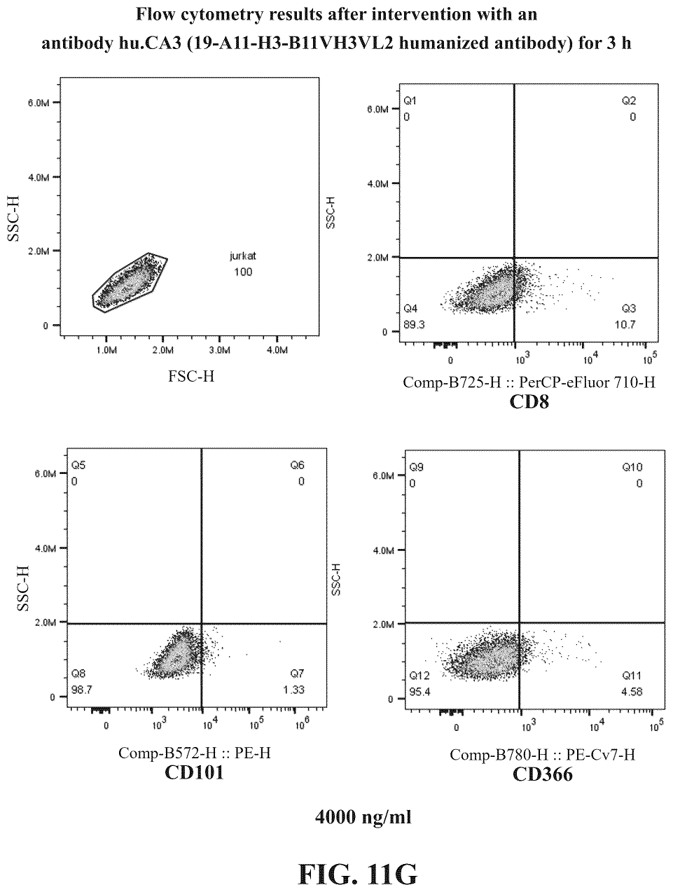

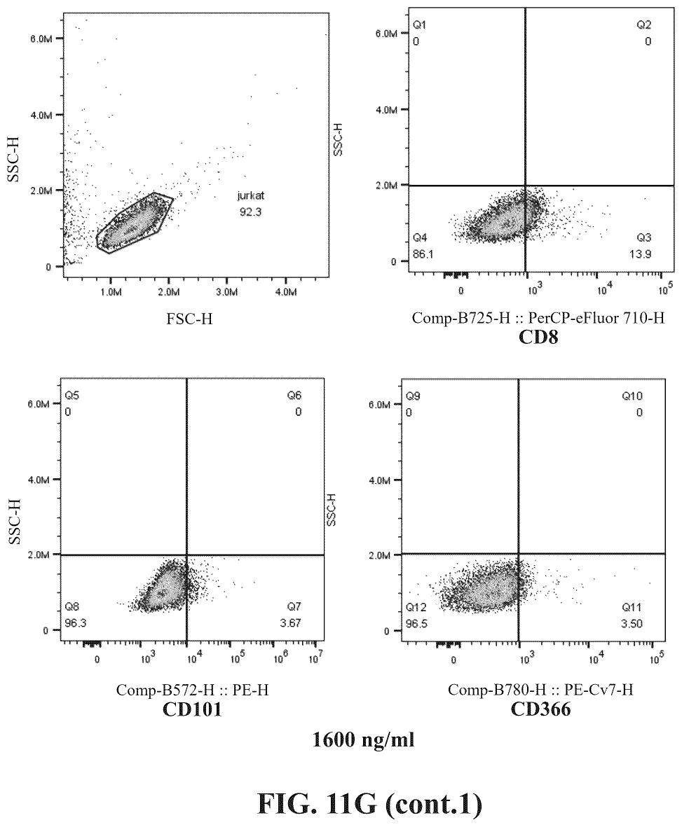

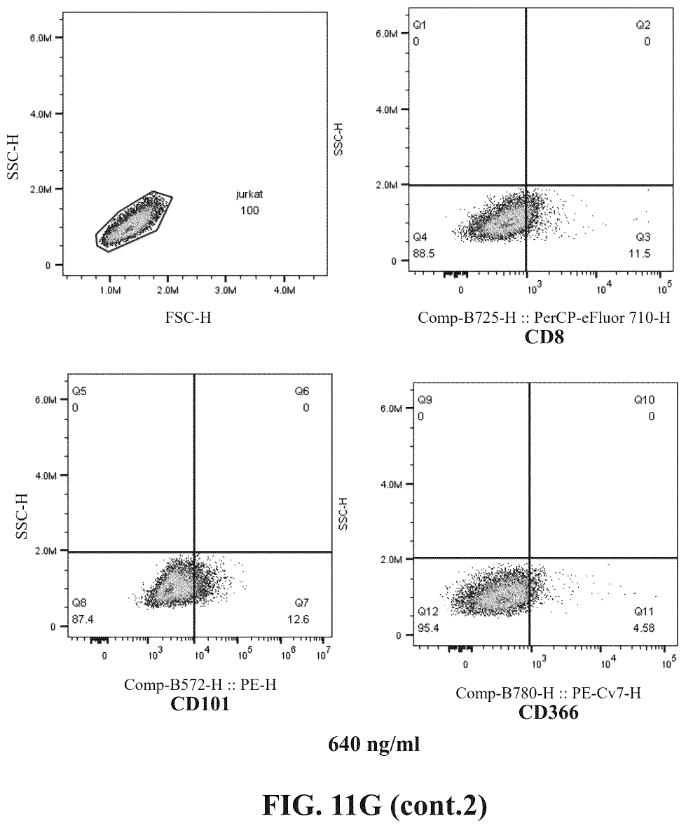

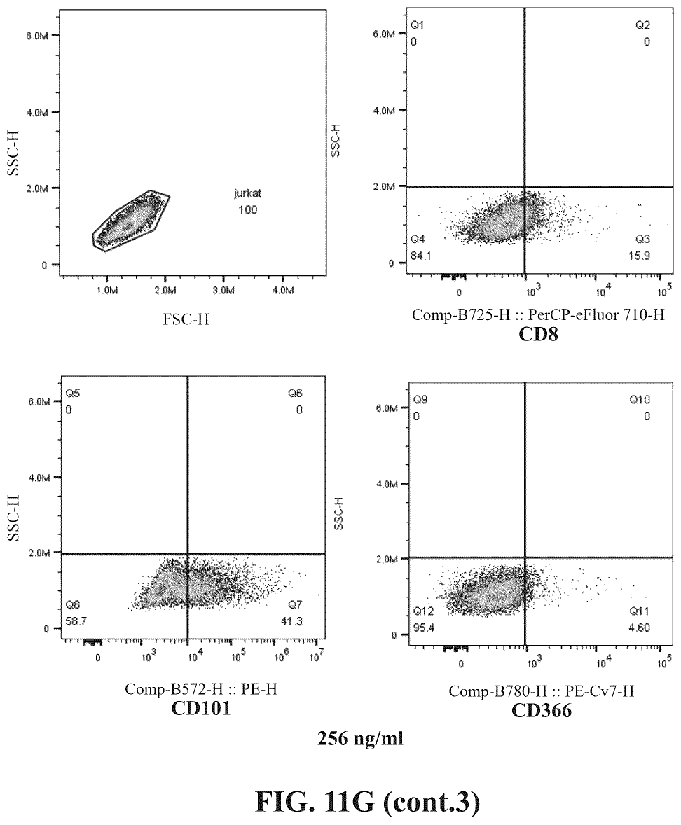

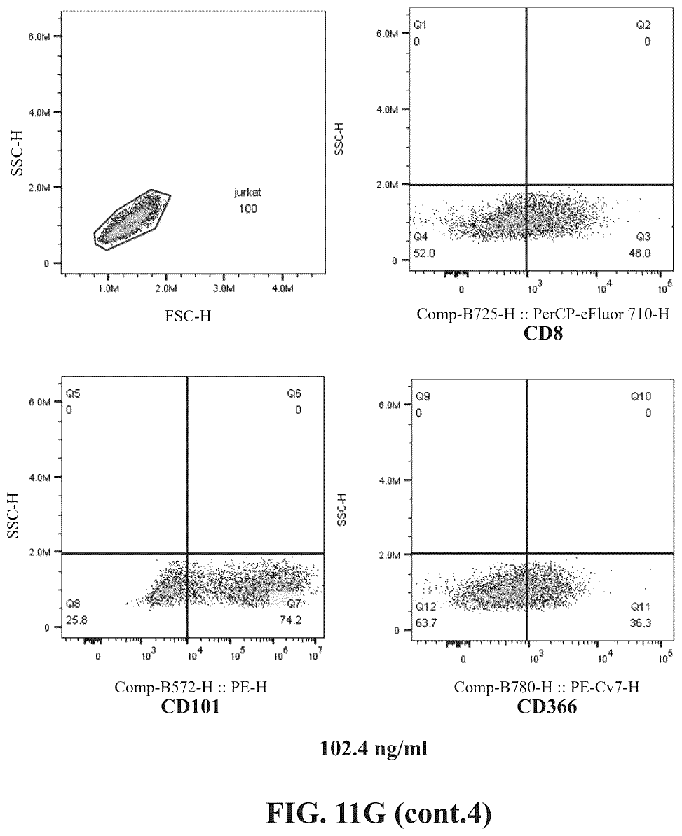

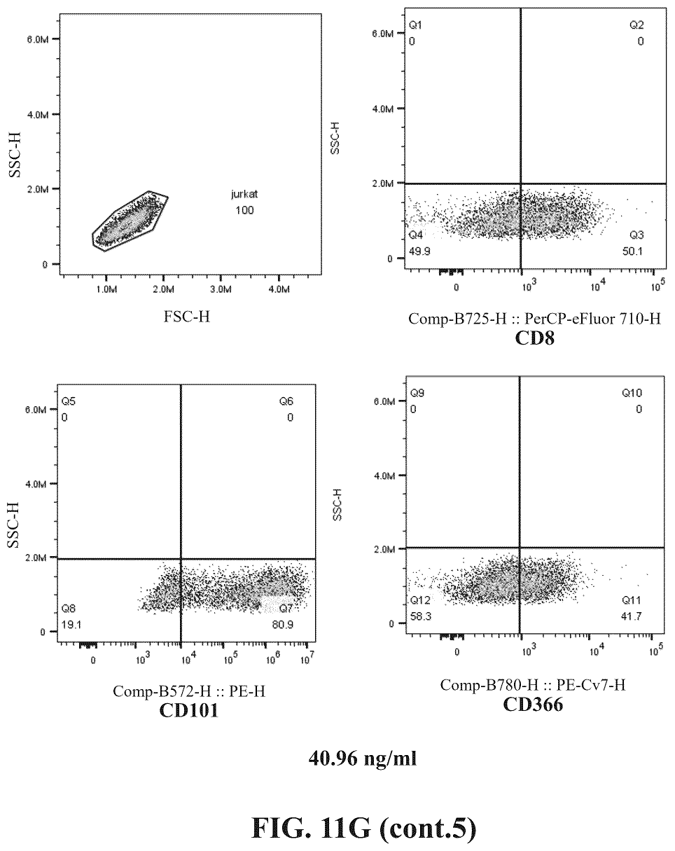

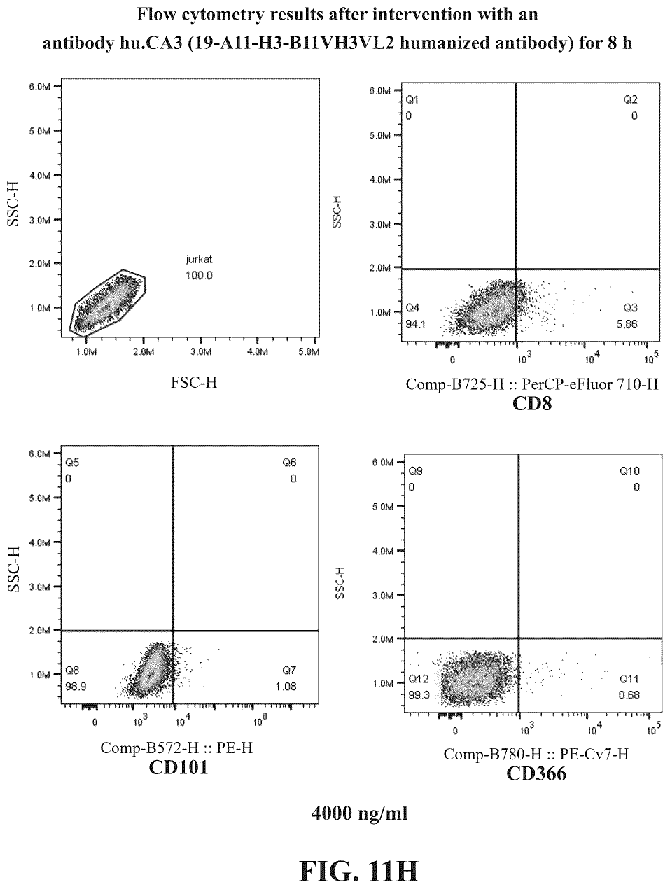

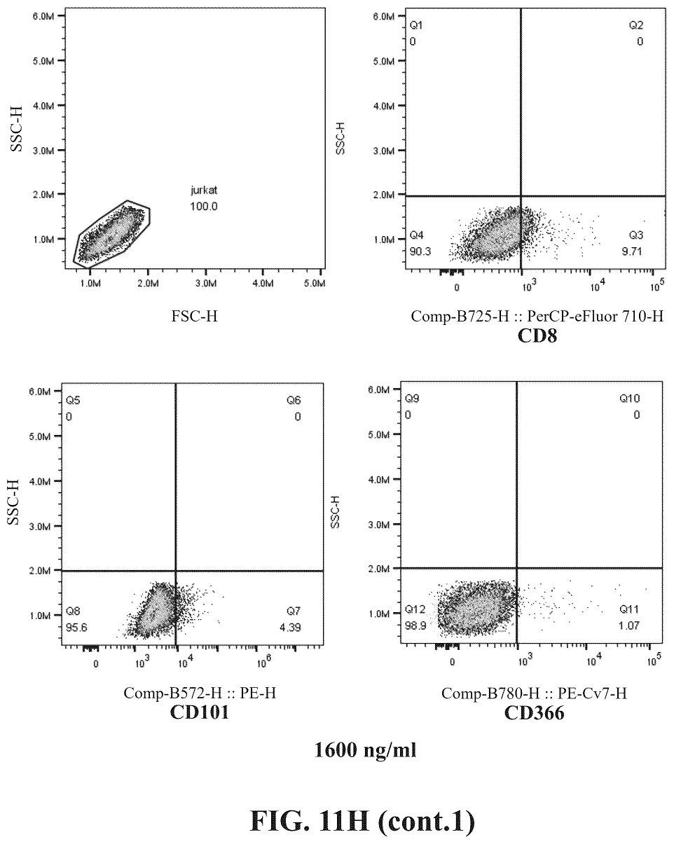

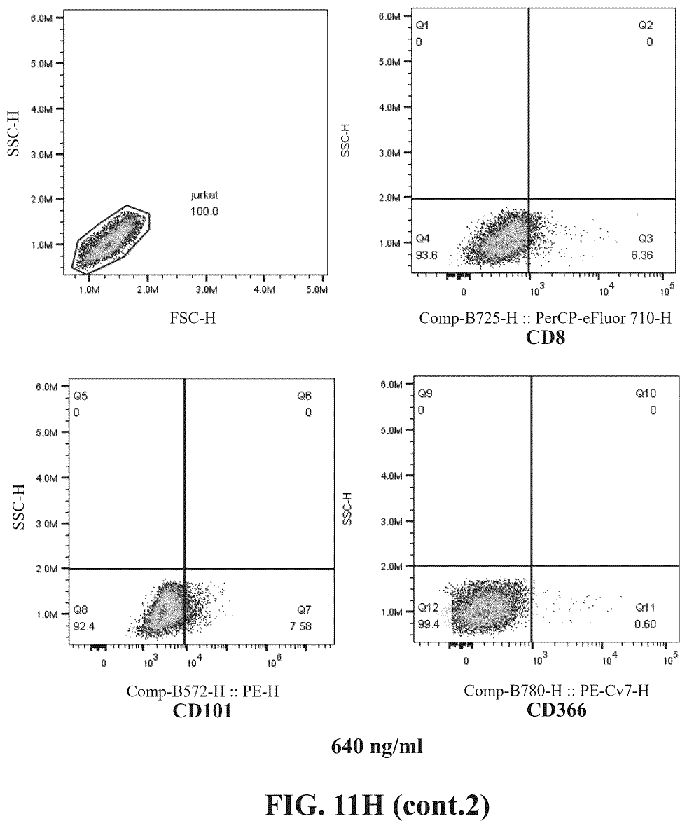

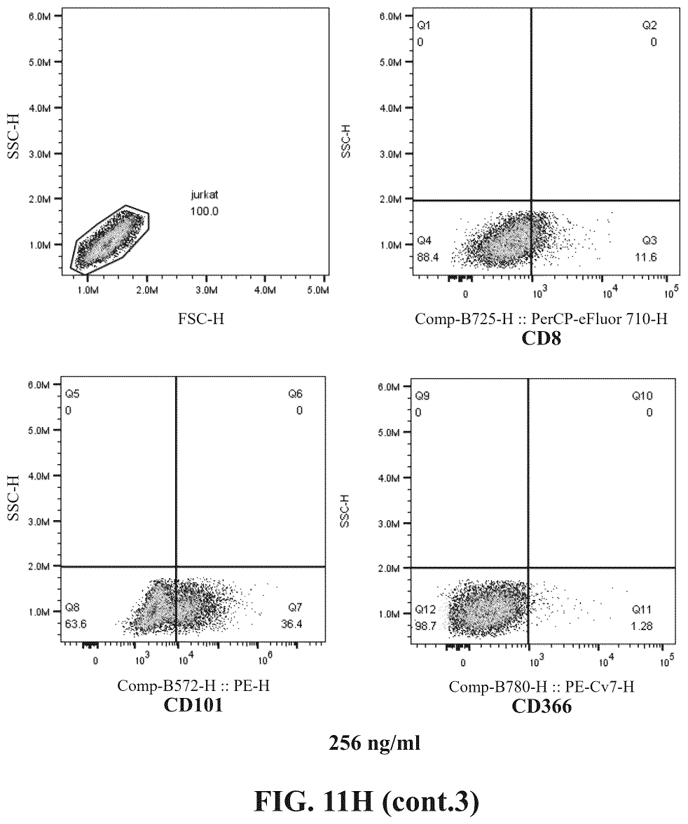

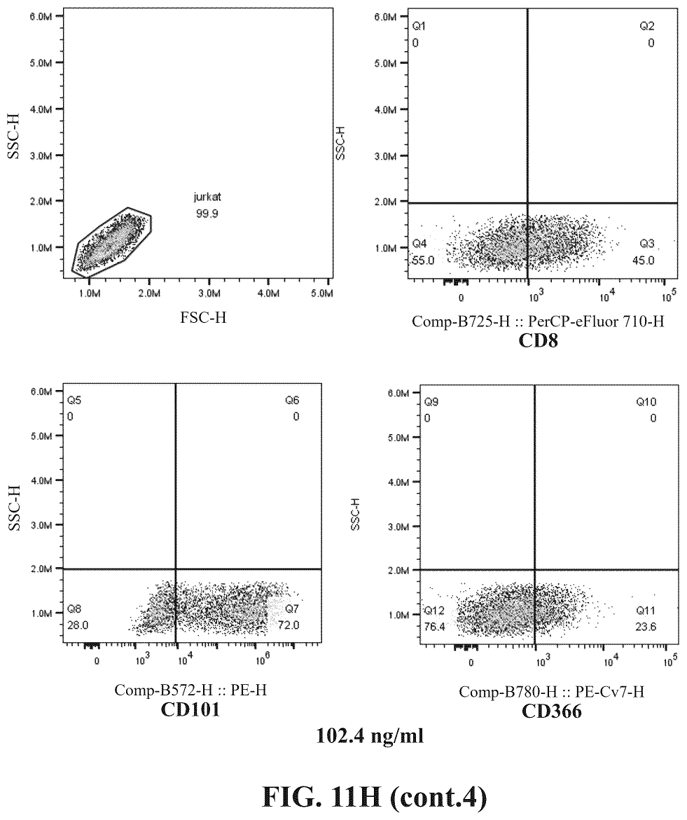

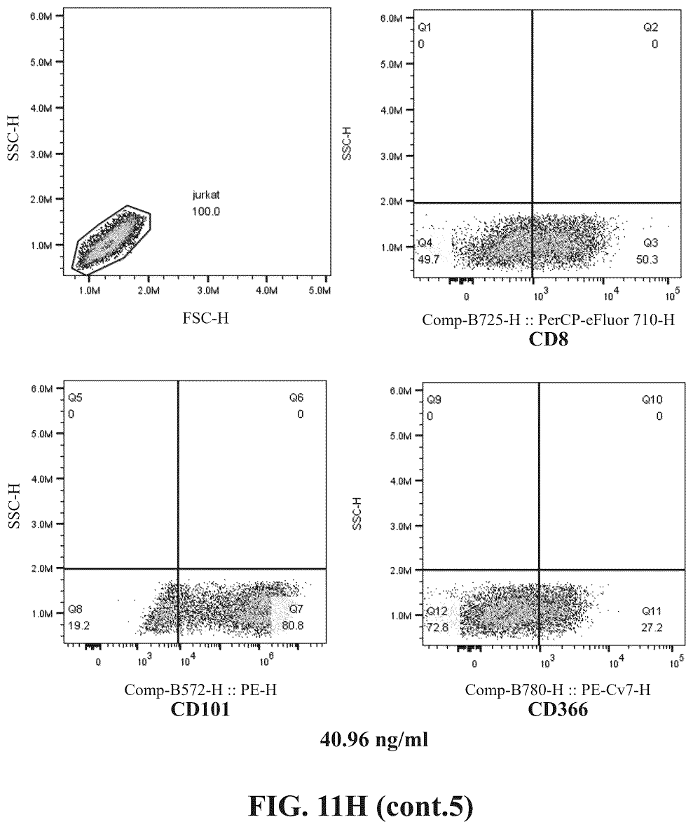

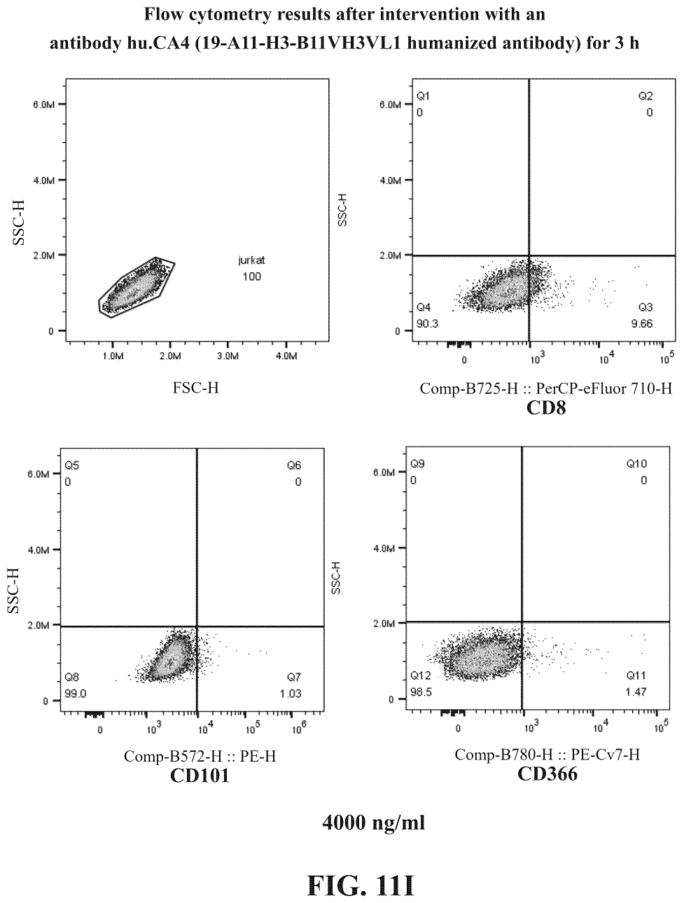

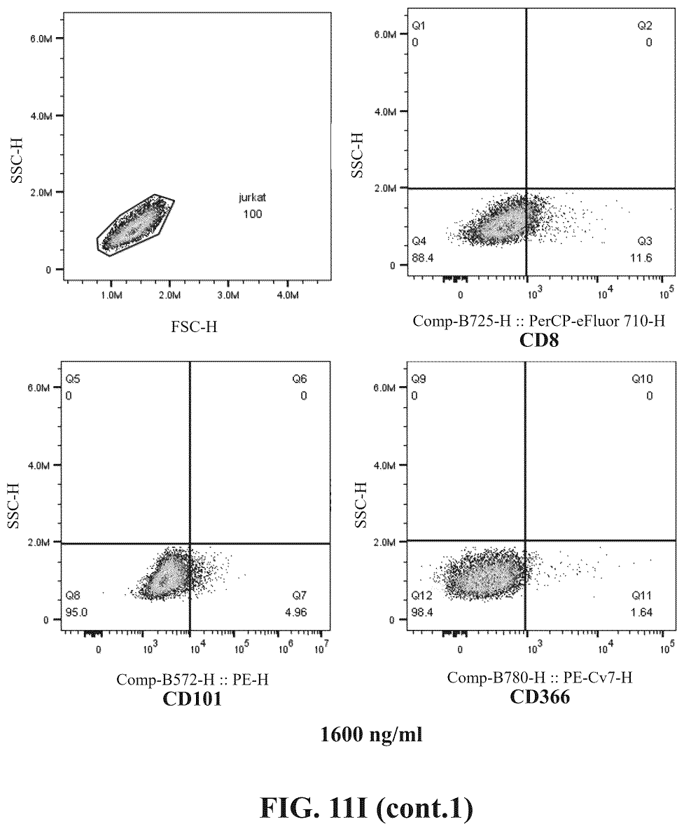

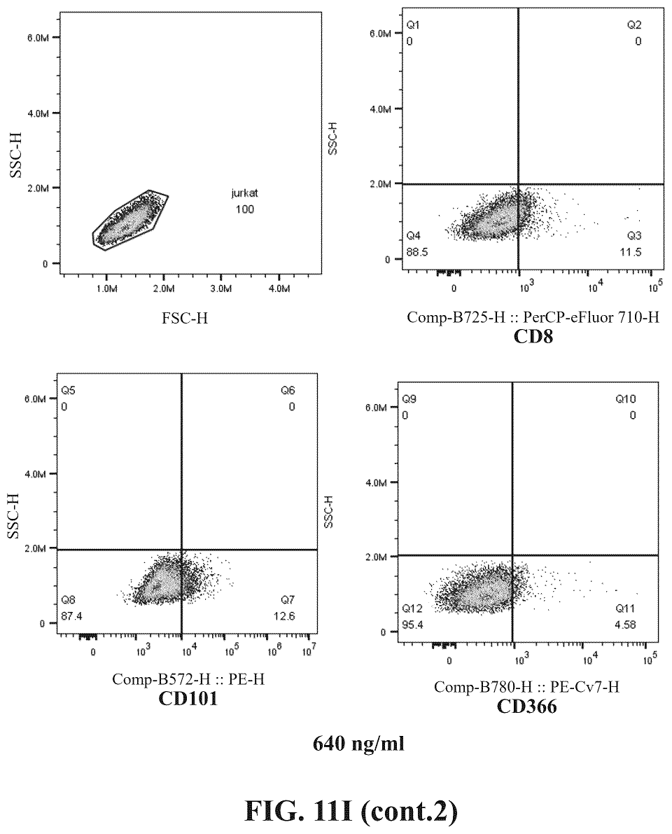

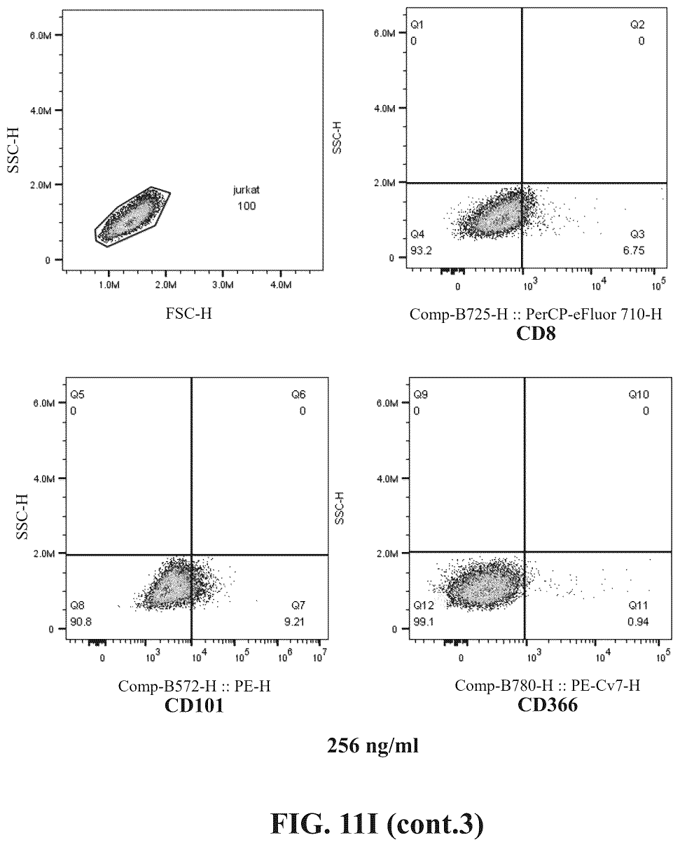

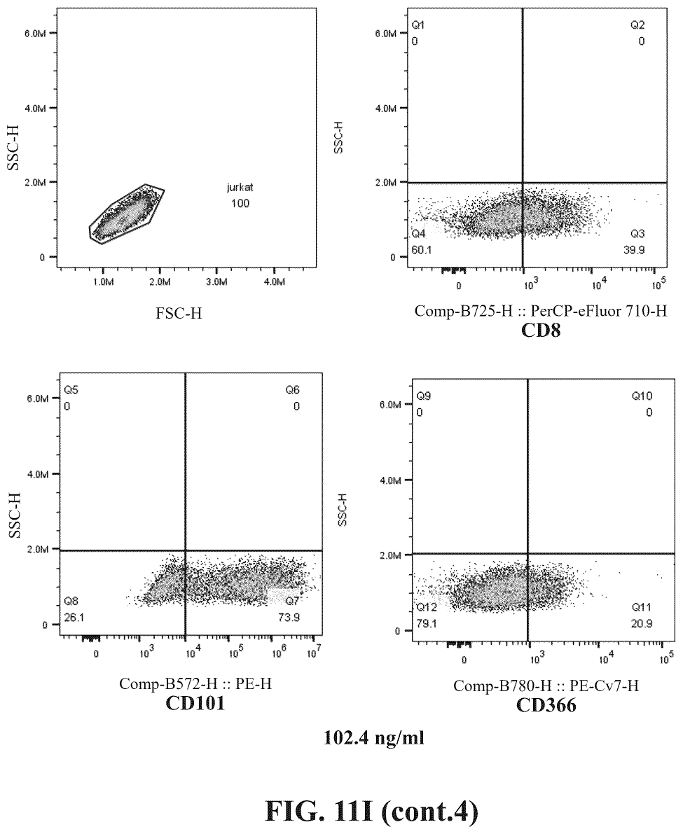

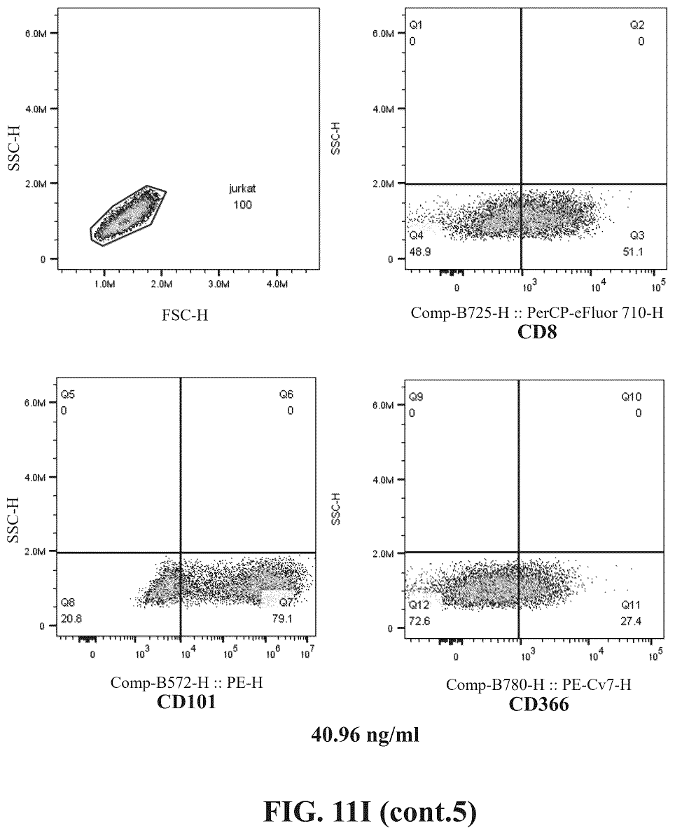

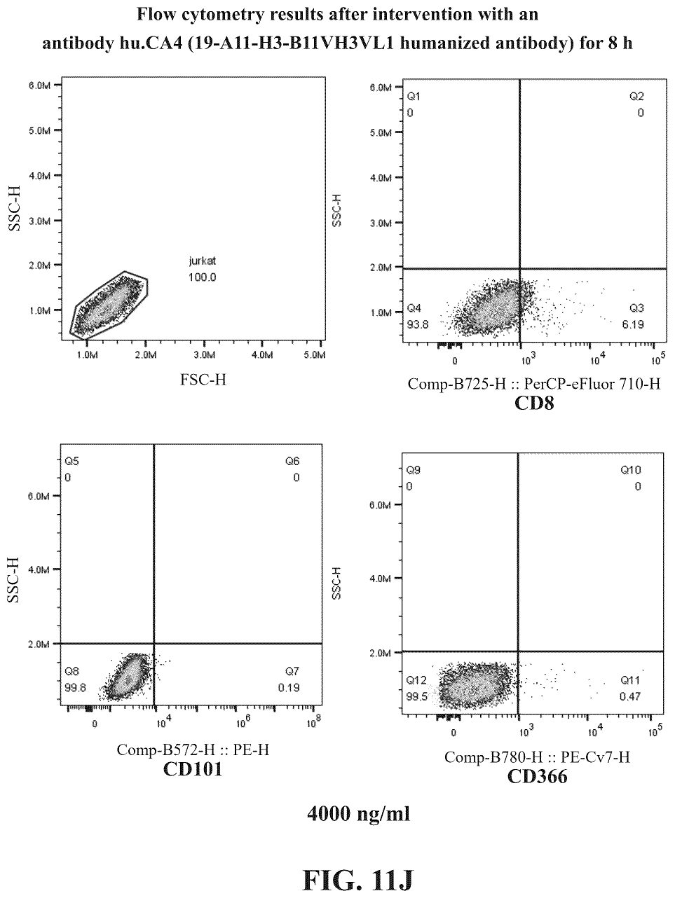

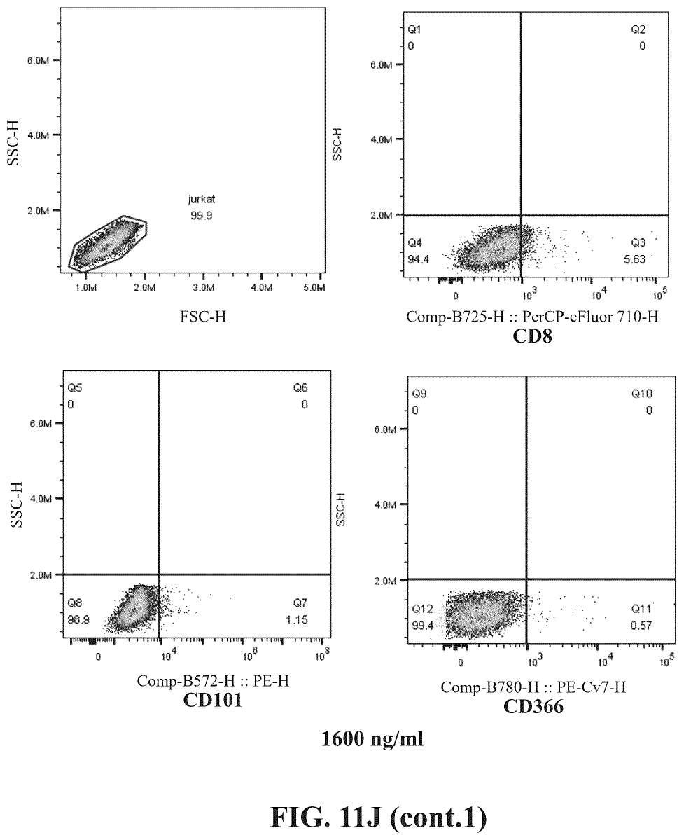

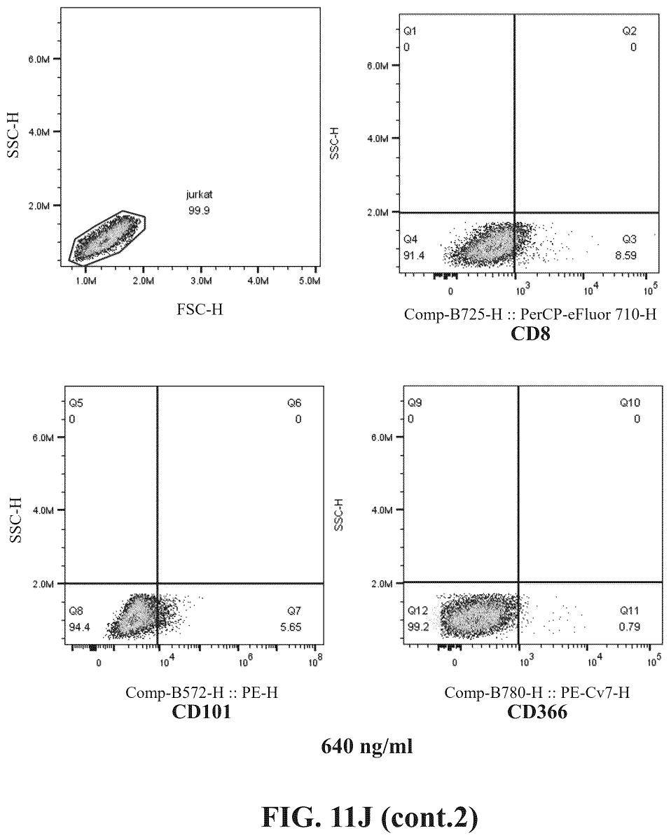

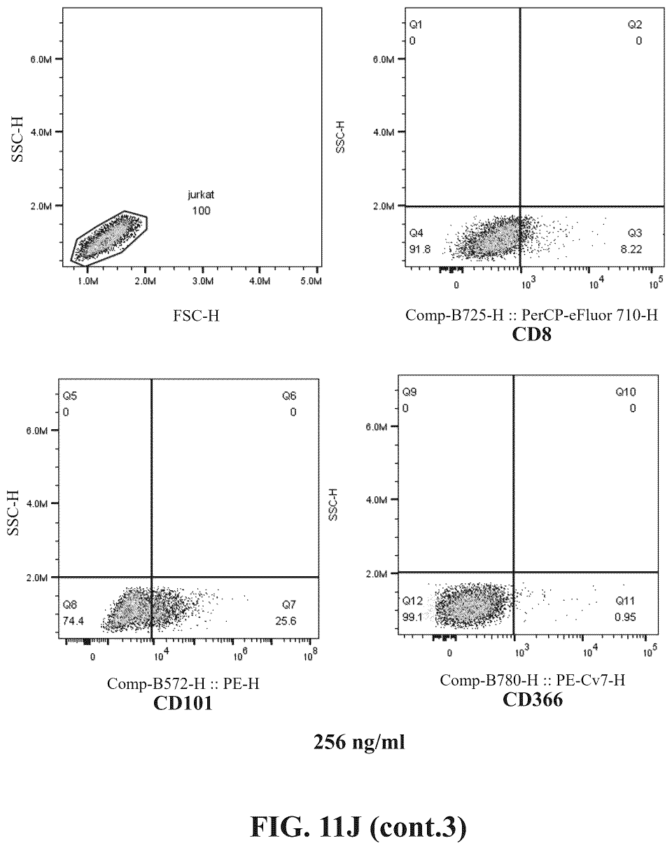

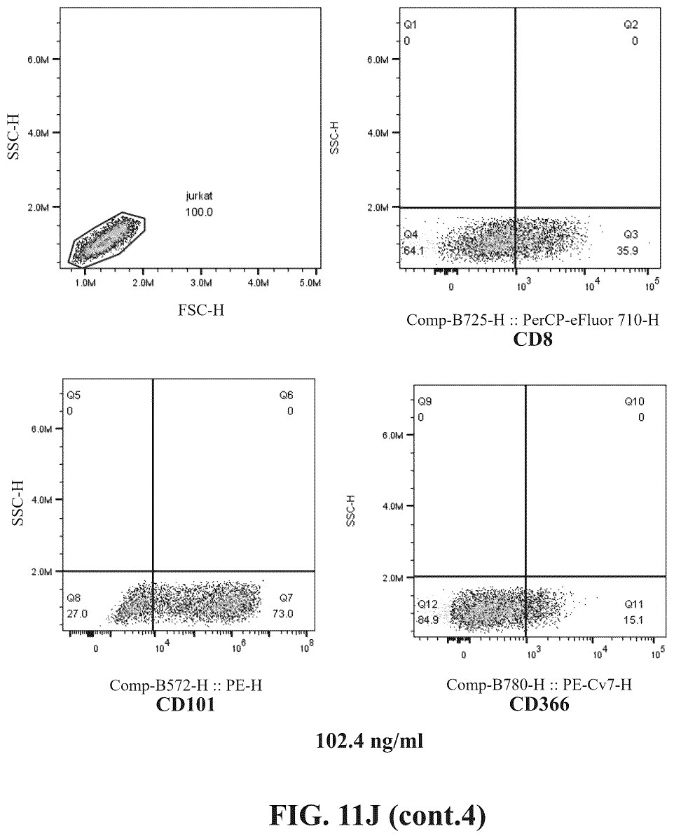

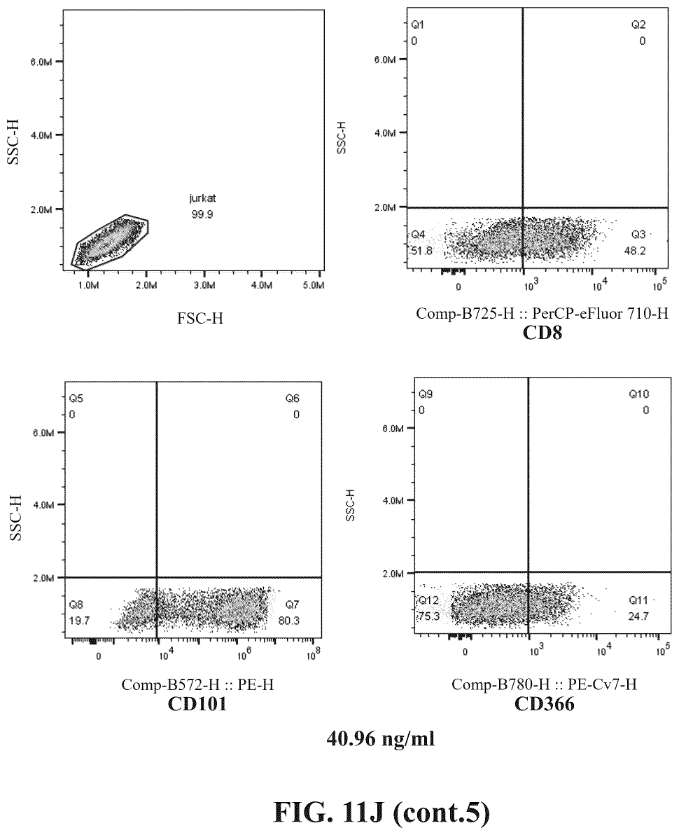

According to the above method, intervention effects of each antibody to be tested at 3 h and 8 h each were tested in the functional experiment of inhibition of a target antigen with the chimeric antibody and the humanized antibodies to be tested. Test results were shown in FIG. 11A to FIG. 11J. The results showed that, after the intervention was conducted with the chimeric antibody and the humanized antibodies for 3 h and 8 h, different concentrations of each antibody to be tested all exhibited a significant inhibitory effect on CD101. At 3 h of intervention (FIG. 11A, FIG. 11C, FIG. 11E, FIG. 11G, and FIG. 11I), different antibodies to be tested at 1,600 ng/ml could exhibit an inhibitory effect of about 80% for CD101, and could also reduce the expression of CD366 (TIM3). TIM3 is one of the recognized biomarkers for T cell exhaustion. After the intervention was conducted with the chimeric antibody and the humanized antibodies for 8 h, each antibody to be tested at 1,600 ng/mL could exhibit an inhibitory effect of 90% or more for CD101, and could significantly reduce a proportion of exhausted CD8⁺ T cells.

The above results showed that an anti-CD101 chimeric antibody and humanized antibodies all exhibited significant affinity and inhibition for CD101 at different concentrations and under different action times. In the follow-up experiments, impacts of the chimeric antibody and the humanized antibodies on functions of immune cells were detected with an action time of 8 h at a concentration of 1,600 ng/ml as an example.

### Example 5 Impacts of a Chimeric Antibody and Humanized Antibodies on Functions of Immune Cells

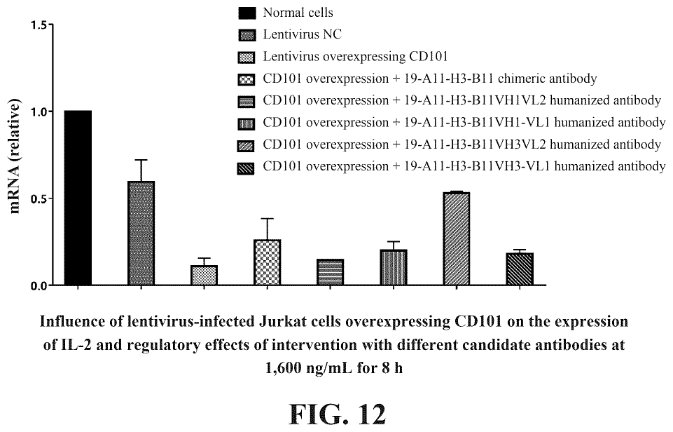

The stable Jurkat cell line carrying a CD101 target prepared above was inoculated at 1.5×10⁶ cells/2 mL/well into a 6-well plate and incubated in a 37° C. incubator for 24 h. Then a chimeric antibody (CA1) and humanized antibodies (hu.CA1 to hu.CA4) each were added at a concentration of 1,600 ng/mL. The plate was further incubated in a 37° C. incubator for 8 h. RNA was extracted with a total RNA extraction kit (Solarbio®, R1200, 50T) and detected by qPCR using TB Green® Fast qPCR Mix (TAKARA®, R045A, 100 Rxns), with a detection index of IL-2. The expression of IL-2 was normalized relative to untransfected normal Jurkat cells (the “normal cells” shown in FIG. 12). The “lentivirus NC” and “lentivirus overexpressing CD101” corresponded to stable Jurkat cells infected with the NC lentivirus PMT290 and lentivirus PSE5799 overexpressing the human CD101 prepared in Example 4, respectively.

An expression level of IL-2, as a detection index, can reflect an activation status of T cells. In many cancers, the function of T cells is inhibited, which is manifested by the reduction in an expression level of IL-2. Therefore, the detection of an expression level of IL-2 can help assess an immune status of a patient and an effect of immunotherapy, and an elevated expression level of IL-2 indicates the restoration of the function of T cells. IL-2 can enhance an anti-tumor immune response in a host by stimulating the proliferation and activation of T cells and NK cells.

Test results were shown in FIG. 12. Expression levels of IL-2 in the chimeric antibody (CA1) and humanized antibody (hu.CA1 to hu.CA4) groups all were higher than an expression level of IL-2 in the lentivirus overexpressing CD101 group, and the humanized antibody hu.CA4 exhibited the optimal effect.

### Example 6 Inhibition of Chimeric Antibodies and Humanized Antibodies on the Growth of Tumors in Mice

### Materials and Methods

5-week-old female SPF-grade C57BL/6J mice used in an experiment were purchased from Guangdong Sijiajingda Biotechnology Co., Ltd. After passing a health examination, the mice were quarantined for 3 d to 7 d and then used for the experiment. The animals had free access to food and drinking water when raised. The animal feeding and management were conducted in accordance with standard operating procedures (SOP) of the Guangdong Specialized School of Traditional Chinese Medicine and regulations of the Guangdong Province on the management of laboratory animals, and all operations and activities involved in the use and welfare of animals were approved by the institutional animal ethics committee before operations.

A time when the administration was started was recorded as day 0 (DO). Mice were inoculated with a tumor (mouse Lewis lung cancer (LLC) cells, National Biomedical Experimental Cell Repository, 1101MOU-PUMC000673, 5×10⁵ cells/mouse) on day 7 (D7) before the start of administration. Then, starting from DO, mice were administered with PBS (100 μL/mouse, N=5), an anti-CD101 antibody (19-A11-H3-B11-VH3VL2 humanized antibody (hu.CA4), 100 μg/mouse (100 μL), N=5), or an anti-PD-1 antibody (BE0146-InVivoMAb anti-mouse PD-1 (CD279), 200 μg/mouse (100 μL), N=5) once every three days through intraperitoneal single-point injection. The anti-CD101 antibody and the anti-PD-1 antibody were stored at −80° C., and thawed and freshly prepared as needed on the day of administration. During the experiment, before each administration, a body weight of each mouse was measured and a tumor size was measured with a vernier caliper. On day 18 (D18), mice were sacrificed, subcutaneous tumors were collected, and the heart, liver, kidney, and thymus were collected through dissection. The organs were weighed.

### Results

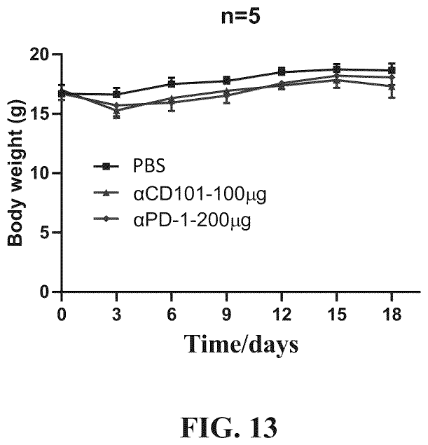

During the experiment, none of the animals died, and there were no abnormalities in a mental state, behaviors, eating, and drinking. Body weights of mice were shown in FIG. 13. There was no significant difference in a mouse body weight between the anti-CD101 antibody experimental group and the PBS control group, indicating that the anti-CD101 antibody exhibited no toxic and side effects for mice and had high safety.

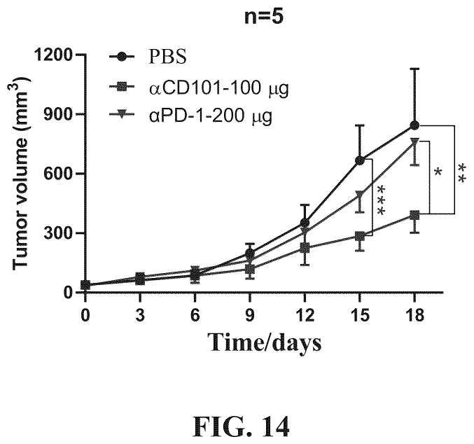

Real-time measurement results of tumors were shown in FIG. 14. The results showed that the anti-CD101 antibody group administered at 100 μg/mouse exhibited a significant inhibitory effect on the tumor growth from day 12 compared with the PBS control group, and had a more significant inhibitory effect than the anti-PD-1 antibody administered at 200 μg/mouse.

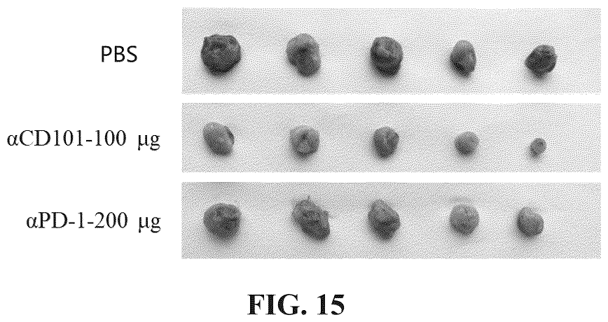

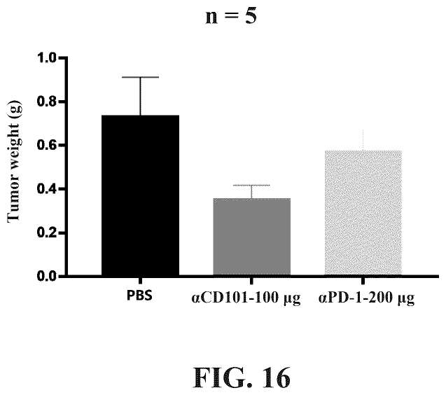

Pictures and weights of tumors collected on D18 were shown in FIG. 15 and FIG. 16, respectively. The results showed that the anti-CD101 antibody (dose: 100 μg/mouse) significantly inhibited the tumor growth compared with the PBS control group, and exhibited a better inhibitory effect than the anti-PD-1 antibody (dose: 200 μg/mouse).

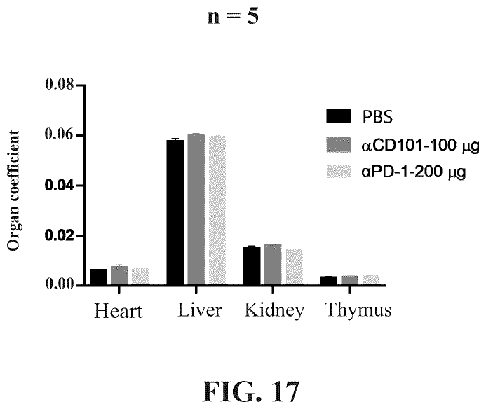

Organ coefficients were shown in FIG. 17. The results showed that the anti-CD101 antibody did not have a significant impact on an organ coefficient, indicating that the anti-CD101 antibody exhibited no obvious toxic and side effects for organs and had high safety.

1. A method for treating a disease in a subject, comprising administering an inhibitor for inhibiting expression and/or activity of CD101 to the subject, wherein the disease is selected from the group consisting of cancer, an inflammatory or infectious disease, an autoimmune disease, or an endocrine disease.

2. The method according to claim 1, wherein the cancer is selected from the group consisting of a hematologic cancer and a solid tumor; optionally, the hematologic cancer is selected from the group consisting of leukemia, polycythemia vera, lymphoma, multiple myeloma, Waldenstrom macroglobulinemia, or heavy chain diseases; and optionally, the solid tumor is selected from the group consisting of lung cancer, melanoma, ovarian cancer, pancreatic ductal adenocarcinoma, liver cancer, colorectal cancer, glioblastoma, or prostate cancer.

3. The method according to claim 1, wherein the cancer is non-small cell lung cancer (NSCLC).

4. The method according to claim 1, wherein the subject does not respond to treatment with a PD-1 axis inhibitor.

5. The method according to claim 1, wherein the inflammatory or infectious disease is selected from the group consisting of acute lung injury (ALI), inflammatory bowel disease (IBD), and human immunodeficiency virus (HIV) infection.

6. The method according to claim 1, wherein the autoimmune disease or the endocrine disease is selected from the group consisting of rheumatoid arthritis (RA) and type 1 diabetes (T1D).

7. The method according to claim 1, wherein the inhibitor for inhibiting expression and/or activity of CD101 is an antibody specifically binding to CD101 or an antigen-binding fragment thereof.

8. The method according to claim 7, wherein the antibody specifically binding to CD101 or the antigen-binding fragment thereof comprises a heavy chain variable region (VH) and a light chain variable region (VL), wherein
(i) the VH comprises CDR-H1, CDR-H2, and CDR-H3 having amino acid sequences set forth in SEQ ID NOs: 1-3, respectively, and the VL comprises CDR-L1, CDR-L2, and CDR-L3 having an amino acid sequence set forth in SEQ ID NO: 5, an amino acid sequence QMS, and an amino acid sequence set forth in SEQ ID NO: 6, respectively; or
(ii) the VH comprises CDR-H1, CDR-H2, and CDR-H3 having amino acid sequences set forth in SEQ ID NOs: 8-10, respectively, and the VL comprises CDR-L1, CDR-L2, and CDR-L3 having an amino acid sequence set forth in SEQ ID NO: 12, an amino acid sequence AAT, and an amino acid sequence set forth in SEQ ID NO: 13, respectively.

9. The method according to claim 8, wherein
(i) the VH comprises an amino acid sequence having at least 80%, at least 85%, at least 90%, at least 95%, at least 98%, at least 99%, or 100% sequence identity to SEQ ID NO: 4, and the VL comprises an amino acid sequence having at least 80%, at least 85%, at least 90%, at least 95%, at least 98%, at least 99%, or 100% sequence identity to SEQ ID NO: 7; or
(ii) the VH comprises an amino acid sequence having at least 80%, at least 85%, at least 90%, at least 95%, at least 98%, at least 99%, or 100% sequence identity to SEQ ID NO: 11, and the VL comprises an amino acid sequence having at least 80%, at least 85%, at least 90%, at least 95%, at least 98%, at least 99%, or 100% sequence identity to SEQ ID NO: 14.

10. The method according to claim 9, wherein
(i) the VH comprises an amino acid sequence set forth in SEQ ID NO: 4, and the VL comprises an amino acid sequence set forth in SEQ ID NO: 7; or
(ii) the VH comprises an amino acid sequence set forth in SEQ ID NO: 11, and the VL comprises an amino acid sequence set forth in SEQ ID NO: 14.

11. The method according to claim 8, wherein
the VH comprises an amino acid sequence having at least 80%, at least 85%, at least 90%, at least 95%, at least 98%, at least 99%, or 100% sequence identity to an amino acid sequence set forth in any one of SEQ ID NOs: 15-18, and the VL comprises an amino acid sequence having at least 80%, at least 85%, at least 90%, at least 95%, at least 98%, at least 99%, or 100% sequence identity to an amino acid sequence set forth in any one of SEQ ID NOs: 19-21.

12. The method according to claim 11, wherein
the VH comprises an amino acid sequence set forth in any one of SEQ ID NOs: 15-18, and the VL comprises an amino acid sequence set forth in any one of SEQ ID NOs: 19-21.

13. The method according to claim 11, wherein
(i) the VH comprises an amino acid sequence set forth in SEQ ID NO: 15, and the VL comprises an amino acid sequence set forth in SEQ ID NO: 19; or
(ii) the VH comprises an amino acid sequence set forth in SEQ ID NO: 15, and the VL comprises an amino acid sequence set forth in SEQ ID NO: 20; or
(iii) the VH comprises an amino acid sequence set forth in SEQ ID NO: 17, and the VL comprises an amino acid sequence set forth in SEQ ID NO: 19; or
(iv) the VH comprises an amino acid sequence set forth in SEQ ID NO: 17, and the VL comprises an amino acid sequence set forth in SEQ ID NO: 20.

14. The method according to claim 1, further comprising administering a second therapeutic agent to the subject, wherein the second therapeutic agent is selected from the group consisting of an antibody, a chemotherapeutic agent, and a small molecule drug.

15. An antibody specifically binding to CD101 or an antigen-binding fragment thereof, comprising VH and VL, wherein
(i) the VH comprises CDR-H1, CDR-H2, and CDR-H3 having amino acid sequences set forth in SEQ ID NOs: 1-3, respectively, and the VL comprises CDR-L1, CDR-L2, and CDR-L3 having an amino acid sequence set forth in SEQ ID NO: 5, an amino acid sequence QMS, and an amino acid sequence set forth in SEQ ID NO: 6, respectively; or
(ii) the VH comprises CDR-H1, CDR-H2, and CDR-H3 having amino acid sequences set forth in SEQ ID NOs: 8-10, respectively, and the VL comprises CDR-L1, CDR-L2, and CDR-L3 having an amino acid sequence set forth in SEQ ID NO: 12, an amino acid sequence AAT, and an amino acid sequence set forth in SEQ ID NO: 13, respectively.

16. The antibody or the antigen-binding fragment thereof according to claim 15, wherein
(i) the VH comprises an amino acid sequence having at least 80%, at least 85%, at least 90%, at least 95%, at least 98%, at least 99%, or 100% sequence identity to SEQ ID NO: 4, and the VL comprises an amino acid sequence having at least 80%, at least 85%, at least 90%, at least 95%, at least 98%, at least 99%, or 100% sequence identity to SEQ ID NO: 7; or
(ii) the VH comprises an amino acid sequence having at least 80%, at least 85%, at least 90%, at least 95%, at least 98%, at least 99%, or 100% sequence identity to SEQ ID NO: 11, and the VL comprises an amino acid sequence having at least 80%, at least 85%, at least 90%, at least 95%, at least 98%, at least 99%, or 100% sequence identity to SEQ ID NO: 14.

17. The antibody or the antigen-binding fragment thereof according to claim 16, wherein
(i) the VH comprises an amino acid sequence set forth in SEQ ID NO: 4, and the VL comprises an amino acid sequence set forth in SEQ ID NO: 7; or
(ii) the VH comprises an amino acid sequence set forth in SEQ ID NO: 11, and the VL comprises an amino acid sequence set forth in SEQ ID NO: 14.

18. The antibody or the antigen-binding fragment thereof according to claim 15, wherein
the VH comprises an amino acid sequence having at least 80%, at least 85%, at least 90%, at least 95%, at least 98%, at least 99%, or 100% sequence identity to an amino acid sequence set forth in any one of SEQ ID NOs: 15-18, and the VL comprises an amino acid sequence having at least 80%, at least 85%, at least 90%, at least 95%, at least 98%, at least 99%, or 100% sequence identity to an amino acid sequence set forth in any one of SEQ ID NOs: 19-21.

19. The antibody or the antigen-binding fragment thereof according to claim 18, wherein
the VH comprises an amino acid sequence set forth in any one of SEQ ID NOs: 15-18, and the VL comprises an amino acid sequence set forth in any one of SEQ ID NOs: 19-21.

20. The antibody or the antigen-binding fragment thereof according to claim 18, wherein
(i) the VH comprises an amino acid sequence set forth in SEQ ID NO: 15, and the VL comprises an amino acid sequence set forth in SEQ ID NO: 19; or
(ii) the VH comprises an amino acid sequence set forth in SEQ ID NO: 15, and the VL comprises an amino acid sequence set forth in SEQ ID NO: 20; or
(iii) the VH comprises an amino acid sequence set forth in SEQ ID NO: 17, and the VL comprises an amino acid sequence set forth in SEQ ID NO: 19; or
(iv) the VH comprises an amino acid sequence set forth in SEQ ID NO: 17, and the VL comprises an amino acid sequence set forth in SEQ ID NO: 20.

21. The antibody or the antigen-binding fragment thereof according to claim 15, wherein the antibody is selected from the group consisting of IgG, IgA, IgM, IgE, and IgD isotypes, wherein the IgG isotype is selected from the group consisting of IgG1, IgG2, IgG3, and IgG4 subtypes.

22. The antibody or the antigen-binding fragment thereof according to claim 15, wherein the antibody is selected from the group consisting of a murine antibody, a chimeric antibody, a humanized antibody, and a fully human antibody.

23. The antibody or the antigen-binding fragment thereof according to claim 15, wherein the antigen-binding fragment is selected from the group consisting of Fab, Fab′, F(ab′)₂, Fv, scFv, and ds-scFv.

24. The antibody or the antigen-binding fragment thereof according to claim 15, wherein the antibody is a bispecific antibody or a multispecific antibody.

25. A nucleic acid comprising a nucleotide sequence encoding the antibody or the antigen-binding fragment thereof according to claim 15.

26. A vector comprising a nucleotide sequence encoding the antibody or the antigen-binding fragment thereof according to claim 15.

27. A host cell comprising the nucleic acid according to claim 25.

28. A host cell comprising the vector according to claim 26.

29. A pharmaceutical composition, comprising: the antibody or the antigen-binding fragment thereof according to claim 15, and a pharmaceutically acceptable carrier or excipient.

30. The pharmaceutical composition according to claim 29, further comprising a second therapeutic agent, wherein the second therapeutic agent is selected from the group consisting of an antibody, a chemotherapeutic agent, and a small molecule drug.


SEQUENCE LISTING (21 sequences)

SEQ ID NO: 1 - protein, 8 aa, synthetic construct
GFTLTEYT

SEQ ID NO: 2 - protein, 8 aa, synthetic construct
FYPGSGGI

SEQ ID NO: 3 - protein, 14 aa, synthetic construct
ARHAFYRNYEYFDV

SEQ ID NO: 4 - protein, 121 aa, synthetic construct
EVQLQQSGAELVRPGASVKLSCKASGFTLTEYTIHWVKQRSGQGLEWIGWFYPGSGGIKYNEKFKDKATLTADESSSTVYMELSRLTSEDSAVYFCARHAFYRNYEYFDVWGTGTTVTVSS

SEQ ID NO: 5 - protein, 11 aa, synthetic construct
KSLLHSNGITY

SEQ ID NO: 6 - protein, 9 aa, synthetic construct
AQNLELPLT

SEQ ID NO: 7 - protein, 112 aa, synthetic construct
DIVMTQAAFSNPVTLGTSASISCRSSKSLLHSNGITYLYWYLQKPGQSPQLLIYQMSNLASGVPDRFSSSGSGTDFTLRISRVEAEDVGVYYCAQNLELPLTFGAGTKLELK

SEQ ID NO: 8 - protein, 8 aa, synthetic construct
GYAFTHYF

SEQ ID NO: 9 - protein, 8 aa, synthetic construct
ITPGSGGT

SEQ ID NO: 10 - protein, 8 aa, synthetic construct
ARGNHGNY

SEQ ID NO: 11 - protein, 115 aa, synthetic construct
EVQLQQSGAELVRPGTSVKVSCKASGYAFTHYFIEWVKQRPGQGLEWIGVITPGSGGTNYNEKFKGKATLTADKSSSTAYMQLSSLTSEDSAVYFCARGNHGNYWGQGTTLTVSS

SEQ ID NO: 12 - protein, 6 aa, synthetic construct
ENIYSN

SEQ ID NO: 13 - protein, 9 aa, synthetic construct
QHFWGNPWT

SEQ ID NO: 14 - protein, 107 aa, synthetic construct
DIQLTQPPASLSVSVGETVTITCRASENIYSNLAWYQQKQGKSPQLLVYAATNLADGVPSRFSGSGSGTQYSLKINSLQSEDFGSYYCQHFWGNPWTFGGGTKLEIK

SEQ ID NO: 15 - protein, 121 aa, synthetic construct
EVQLVQSGAEVKKPGASVKVSCKASGFTLTEYTIHWVRQAPGQGLEWIGWFYPGSGGIKYNEKFQDRVTLTADESISTVYMELSRLRSDDTAVYFCARHAFYRNYEYFDVWGTGTMVTVSS

SEQ ID NO: 16 - protein, 121 aa, synthetic construct
EVQLVQSGAEVKKPGASVKVSCKASGFTLTEYTIHWVRQAPGQGLEWIGWFYPGSGGIKYNEKFQDRVTLTADESASTVYMELSSLRSEDTAVYFCARHAFYRNYEYFDVWGTGTMVTVSS

SEQ ID NO: 17 - protein, 121 aa, synthetic construct
EVQLVQSGAEVKKPGASVKVSCKASGFTLTEYTIHWVRQAPGQGLEWIGWFYPGSGGIKYNEKFQDRVTLTADTSISTVYMELSRLRSDDTVVYFCARHAFYRNYEYFDVWGTGTMVTVSS

SEQ ID NO: 18 - protein, 121 aa, synthetic construct
EVQLVQSGAEVKKPGASVKVSCKASGFTLTEYTIHWVKQAPGQGLEWIGWFYPGSGGIKYNEKFQDRVTLTADESSSTVYMELSRLRSDDTAVYFCARHAFYRNYEYFDVWGTGTMVTVSS

SEQ ID NO: 19 - protein, 112 aa, synthetic construct
DIVMTQAPFSLPVTPGTPASISCRSSKSLLHSNGITYLYWYLQKPGQSPQLLIYQMSNLASGVPDRFSSSGSGTDFTLKISRVEAEDVGVYYCAQNLELPLTFGAGTKLEIK

SEQ ID NO: 20 - protein, 112 aa, synthetic construct
DIVMTQAPLSLPVTPGTPASISCRSSKSLLHSNGITYLYWYLQKPGQSPQLLIYQMSNLASGVPDRFSSSGSGTDFTLKISRVEAEDVGVYYCAQNLELPLTFGAGTKLEIK

SEQ ID NO: 21 - protein, 112 aa, synthetic construct
DIVMTQAPFSSPVTLGTPASISCRSSKSLLHSNGITYLYWYQQRPGQSPRLLIYQMSNLASGVPDRFSSSGSGTDFTLKISRVEAEDVGVYYCAQNLELPLTFGAGTKLEIK